(19) United States
(12) Patent Application Publication
(10) Pub. No.: US 2025/0127782 A1
(43) Pub. Date: Apr. 24, 2025

(54) METHODS OF TREATING CANCER

(71) Applicant: AMGEN INC., Thousand Oaks, CA (US)

(72) Inventors: Emily CHAN, Thousand Oaks, CA (US); Gregory FRIBERG, Westlake Village, CA (US); Omar Ali MATHER, Thousand Oaks, CA (US); Brett E. HOUK, Newbury Park, CA (US); Gataree NGARMCHAMNANRITH, Los Angeles, CA (US); Haby HENARY, Moorpark, CA (US); Sandeep DUTTA, Newbury Park, CA (US)

(21) Appl. No.: 18/689,716

(22) PCT Filed: Sep. 7, 2022
(86) PCT No.: PCT/US2022/076052

§ 371 Date: Mar. 6, 2024

Related U.S. Application Data
(60) Provisional application No. 63/241,601, filed on Sep. 8, 2021.
(60) Provisional application No. 63/298,747, filed on Jan. 12, 2022.
(60) Provisional application No. 63/374,012, filed on Aug. 31, 2022.

(51) Int. Cl. A61K 31/519; A61K 31/4745; A61K 39/395; A61K 45/06; A61P 35/00

(52) U.S. Cl. CPC A61K 31/519; A61K 31/4745; A61K 39/3955; A61K 45/06; A61P 35/00

(57) ABSTRACT

Provided herein are methods of treating cancer comprising a KRAS G12C mutation in a patient comprising administering to the patient sotorasib and an anti-epidermal growth factor receptor (EGFR) antibody in amounts effective to treat the cancer. Further provided herein are methods further comprising administering FOLFIRI (irinotecan, 5-FU and leucovorin) to the patient.

### CROSS-REFERENCE TO RELATED APPLICATIONS

This application claims the benefit of U.S. Provisional Patent Application No. 63/241,601, filed Sep. 8, 2021, U.S. Provisional Patent Application No. 63/298,747, filed Jan. 12, 2022, and U.S. Provisional Patent Application No. 63/374,012, filed Aug. 31, 2022, each of which is incorporated herein by reference in its entirety.

### INCORPORATION BY REFERENCE OF MATERIAL SUBMITTED ELECTRONICALLY

The name of the text file containing the Sequence Listing is “55328P3_Seqlisting.XML”, which was created on Aug. 23, 2022 and is 13,982 bytes in size. The subject matter of the Sequence Listing is incorporated herein in its entirety by reference.

### BACKGROUND

The rat sarcoma (RAS) proto-oncogene has been identified as an oncogenic driver of tumorigenesis in cancers, such as non-small cell lung cancer (NSCLC) and colorectal cancer (CRC). The RAS family consists of 3 closely related genes that express guanosine triphosphate (GTP)-ases responsible for regulating cellular proliferation and survival. The RAS proteins, Kirsten rat sarcoma viral oncogene homolog (KRAS), Harvey rat sarcoma viral oncogene homolog (HRAS), and neuroblastoma RAS viral oncogene homolog (NRAS) can be mutationally activated at codons 12, 13, or 61, leading to human cancers. Different tumor types are associated with mutations in certain isoforms of RAS, with KRAS being the most frequently mutated isoform in most cancers. While the role of KRAS mutations in human cancers has been known for decades, no anti-cancer therapies specifically targeting KRAS mutations have been successfully developed, until recently, largely because the protein had been considered intractable for inhibition by small molecules.

### SUMMARY

Described herein are methods of treating cancer comprising a KRAS G12C mutation in a patient comprising administering to the patient sotorasib and an anti-epidermal growth factor receptor (EGFR) antibody in amounts effective to treat the cancer. In some embodiments, the anti-EGFR antibody comprises the heavy chain HCDR1 of SEQ ID NO: 1, HCDR2 of SEQ ID NO: 2, HCDR3 of SEQ ID NO: 3, light chain LCDR1 of SEQ ID NO: 6, LCDR2 of SEQ ID NO: 7, and LCDR3 of SEQ ID NO: 8. In some embodiments, the anti-EGFR antibody comprises the heavy chain variable region sequence of SEQ ID NO: 4 and the light chain variable region of SEQ ID NO: 9. In some embodiments, the anti-EGFR antibody comprises the heavy chain sequence of SEQ ID NO: 5 and the light chain sequence of SEQ ID NO: 10. In some embodiments, the anti-EGFR antibody is panitumumab.

In some embodiments, the methods further comprise administering irinotecan, 5-fluoro-1H-pyrimidine-2,4-dione (5-fluorouracil, 5-FU) and leucovorin to the patient. In some embodiments, the methods further comprise administering irinotecan, 5-FU and levoleucovorin to the patient. In some embodiments, the methods further comprise administering irinotecan and 5-FU to the patient.

In various embodiments, the cancer is a solid tumor. In various embodiments, the cancer is non-small cell lung cancer, and in some cases, is metastatic or locally advanced. In various embodiments, the cancer is colorectal cancer. In various embodiments, the cancer is pancreatic cancer. In various embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma.

### BRIEF DESCRIPTION OF THE FIGURES

FIG. 1 shows the mean plasma concentration time profile after once daily oral administration of 180, 360, 720, or 960 mg sotorasib on Day 1, where N indicates number of observations across data points.

FIG. 2 shows the mean plasma concentration time profile after once daily oral administration of 180, 360, 720, or 960 mg sotorasib on Day 8, where N indicates number of observations across data points.

### DETAILED DESCRIPTION

Provided herein are methods of treating cancer comprising a KRAS G12C mutation in a patient comprising administering to the patient sotorasib and an anti-epidermal growth factor receptor (EGFR) antibody in amounts effective to treat the cancer. In some embodiments, the methods further comprise administering irinotecan, 5-FU and leucovorin to the patient. In some embodiments, the methods further comprise administering irinotecan, 5-FU and levoleucovorin to the patient. In some embodiments, the methods further comprise administering irinotecan and 5-FU to the patient.

The methods of treatment disclosed herein regarding administration of two or more therapeutics (e.g., sotorasib, EGFR antibody, irinotecan, 5-FU, leucovorin, etc.) to a patient include concomitant administration of the therapeutics (e.g., within 1 hour, within 45 minutes, within 30 minutes, within 15 minutes, or within 10 minutes of each other), and sequential administration (e.g., administration separated by at least 1 hour, or at least two hours, or at least four hours, or at least six hours, or at least eight hours, or at least twelve hours, or at least 24 hours, or at least 2 days, or at least 3 days). Unless otherwise described herein, combination therapy of two or more therapeutics as discussed herein include both concomitant and sequential administration.

### Sotorasib

Sotorasib is a small molecule that irreversibly inhibits the KRAS^{G12C} mutant protein. Sotorasib is also referred to as AMG 510 or 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following structure:

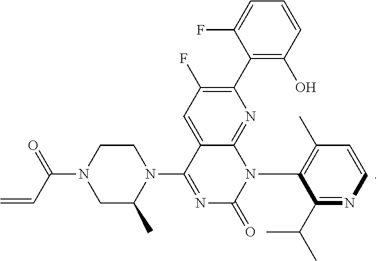

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an inactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019). See also, LUMAKRAS® US Prescribing Information, Amgen Inc., Thousand Oaks, California, 91320 (revision May 2021), which is herein incorporated by reference in its entirety.

### Anti-EGFR Antibody

In some embodiments, the methods further comprise administering an anti-epidermal growth factor receptor (EGFR) antibody to the patient. In some embodiments, the anti-EGFR antibody comprises the heavy chain HCDR1 of SEQ ID NO: 1, HCDR2 of SEQ ID NO: 2, HCDR3 of SEQ ID NO: 3, light chain LCDR1 of SEQ ID NO: 6, LCDR2 of SEQ ID NO: 7, and LCDR3 of SEQ ID NO: 8. In some embodiments, the anti-EGFR antibody comprises the heavy chain variable region sequence of SEQ ID NO: 4 and the light chain variable region of SEQ ID NO: 9. In some embodiments, the anti-EGFR antibody comprises the heavy chain sequence of SEQ ID NO: 5 and the light chain sequence of SEQ ID NO: 10. In some embodiments, the anti-EGFR antibody is panitumumab.

Panitumumab is a fully human immunoglobulin (Ig)G2 monoclonal antibody to the epidermal growth factor receptor (EGFR). Panitumumab binds to the extracellular domain of EGFR, thus preventing its activation and intracellular signaling.

Panitumumab (VECTIBIX®) has been approved for the treatment of patients with wild-type RAS (in both KRAS and NRAS as determined by an FDA approved test for this use) metastatic colorectal cancer (mCRC) as first line therapy in combination with FOLFOX (leucovorin calcium (folinic acid), fluorouracil, and oxaliplatin) and as monotherapy following disease progression after prior treatment with fluoropyrimidine, oxaliplatin and irinotecan-containing chemotherapy. The recommended dose is 6 mg/kg, administered as an IV infusion over 60 minutes (≤1000 mg) or 90 minutes (>1000 mg), Q2W. See also, VECTIBIX® US Prescribing Information, Amgen Inc., Thousand Oaks, California, 91320 (revision August 2021)), which is herein incorporated by reference in its entirety.

### FOLFIRI

In some embodiments, the methods further comprise administering irinotecan, 5-FU and leucovorin to the patient. In some embodiments, the methods further comprise administering irinotecan, 5-FU and levoleucovorin to the patient.

The FOLFIRI regimen consists of irinotecan 180 mg/m² on day 1, racemic leucovorin 400 mg/m² on day 1 and 5-fluorouracil 400 mg/m² IV bolus on day 1 and 2400 mg/m² IV continuous infusion (IVCI) over 46 to 48 hours beginning on day 1 given Q2W (National Comprehensive Cancer Network (NCCN) Colon, Rectal, Anal Cancer Guidelines). Irinotecan in combination with 5-fluourouracil and leucovorin is FDA-approved for first line treatment for patients with metastatic carcinoma of the colon or rectum (CAMPTOSAR® US Prescribing Information, Pharmacia and Upjohn Co., Division of Pfizer, Inc., NY, NY 10017 (revision January 2022), which is herein incorporated by reference in its entirety). In some embodiments, leucovorin in the FOLFIRI regimen may be substituted with 200 mg/m² of levoleucovorin.

### Dosing Regimens

In some embodiments, the methods comprise administering sotorasib in an amount ranging from 240 mg to 960 mg. In some embodiments, the methods comprise administering 960 mg sotorasib to the patient once daily. In some embodiments, the methods comprise administering 720 mg sotorasib to the patient once daily. In some embodiments, the methods comprise administering 480 to the patient once daily. In some embodiments, the methods comprise administering 240 mg to the patient once daily. In some embodiments, the methods comprise administering 480 mg to the patient twice daily. In some embodiments, the methods comprise administering 240 mg to the patient twice daily.

In some embodiments, the methods comprise administering panitumumab to the patient once every two weeks. In some embodiments, the methods comprise administering panitumumab in an amount ranging from 3.0 mg/kg to 6 mg/kg (e.g., 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg·kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, or 6 mg/kg) via IV administration once every two weeks. In some embodiments, the methods comprise administering 6 mg/kg panitumumab. In some embodiments, the methods further comprise administering 3 mg/kg panitumumab.

In some embodiments, the methods described herein comprise administering to the patient (a) 960 mg sotorasib daily; and (b) 6 mg/kg panitumumab via IV administration every two weeks. In some embodiments, the methods described herein comprise administering to the patient (a) 720 mg sotorasib daily; and (b) 6 mg/kg panitumumab via IV administration every two weeks. In some embodiments, the methods described herein comprise administering to the patient (a) 480 mg sotorasib daily; and (b) 6 mg/kg panitumumab via IV administration every two weeks. In some embodiments, the methods described herein comprise administering to the patient (a) 960 mg sotorasib daily; and (b) 3 mg/kg panitumumab via IV administration every two weeks.

In some embodiments, the methods further comprise administering irinotecan, 5-FU and leucovorin to the patient. In some embodiments, the methods comprise administering 400 mg/m² leucovorin via IV administration to the patient. In some embodiments, the methods further comprise administering irinotecan, 5-FU and levoleucovorin to the patient. In some embodiments, the methods further comprise administering 200 mg/m² levoleucovorin via IV administration to the patient. In some embodiments, the methods further comprise administering 180 mg/m² irinotecan via IV administration to the patient. In some embodiments, the methods further comprise administering 400 mg/m² 5-FU via IV administration to the patient.

In some embodiments, the methods further comprise administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

In some embodiments, the methods further comprise administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

In various embodiments, sotorasib is administered with food. In various embodiments, sotorasib is administered without food.

In various embodiments, the patient is in further need of treatment with an acid-reducing agent. Acid-reducing agents include, but are not limited to, a proton pump inhibitor (PPI), a H2 receptor antagonist (H2RA), and a locally acting antacid. In some embodiments, the patient is further in need of treatment with a PPI or a H2RA. Exemplary PPIs include, but are not limited to, omeprazole, pantoprazole, esomeprazole, lansoprazole, rabeprazole, or dexlansoprazole. Exemplary H2RAs include, but are not limited to, famotidine, ranitidine, cimetidine, nizatidine, roxatidine and lafutidine. Exemplary locally acting antacids include, but are not limited to, sodium bicarbonate, calcium carbonate, aluminum hydroxide, and magnesium hydroxide. In some embodiments, the patient, who is in further need of treatment with an acid-reducing agent, is not administered a proton pump inhibitor or a H2 receptor antagonist in combination with sotorasib. In some embodiments, the patient, who is in further need of treatment with an acid-reducing agent, is not administered a proton pump inhibitor or a H2 receptor antagonist in combination with sotorasib, but is administered a locally acting antacid in combination with sotorasib. In some embodiments, sotorasib is administered about 4 hours before or about 10 hours after a locally acting antacid.

In various embodiments, the patient is in further need of treatment with a CYP3A4 inducer. In some embodiments, the patient is not administered a CYP3A4 inducer in combination with sotorasib. Exemplary CYP3A4 inducers include, but are not limited to, barbiturates, brigatinib, carbamazepine, clobazam, dabrafenib, efavirenz, elagolix, enzalutamide, eslicarbazepine, glucocorticoids, letermovir, lorlatinib, modafinil, nevirapine, oritavancin, oxcarbazepine, perampanel, phenobarbital, phenytoin, pioglitazone, rifabutin, rifampin, telotristat, and troglitazone. See, e.g., Flockhart DA, Drug Interactions: Cytochrome P450 Drug Interaction Table. Indiana University School of Medicine (2007), www.drug-interactions.medicine.iu.edu, accessed May 2021. In some embodiments, the patient is not administered a strong CYP3A4 inducer in combination with sotorasib. Exemplary strong CYP3A4 inducers include, but are not limited to, phenytoin and rifampin. See, e.g., www.fda.gov/drugs/drug-interactions-labeling/drug-development-and-drug-interactions-table-substrates-inhibitors-and-inducers, accessed May 2021. In one embodiment strong CYP3A4 inhibitors include, but are not limited to ombitasvir and paritaprevir and ritonavir and dasabuvir, indinavir and ritonavir, tipranavir and ritonavir, ritonavir, cobicistat, ketoconazole, troleandomycin, telaprevir, danoprevir and ritonavir, elvitegravir and ritonavir, saquinavir and ritonavir, lopinavir and ritonavir, itraconazole, indinavir, voriconazole, mifepristone, mibefradil, LCL161, clarithromycin, josamycin, lonafarnib, posaconazole, telithromycin, grapefruit juice DS3, conivaptan, tucatinib, nefazodone, ceritinib, nelfinavir, saquinavir, ribociclib, idelalisib, and boceprevir.

In various embodiments, the patient is in further need of treatment with a CYP3A4 substrate. In some embodiments, the patient is not administered a CYP3A4 substrate in combination with sotorasib. Exemplary CYP3A4 substrates include, but are not limited to, abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus(fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, and ziprasidone. See, e.g., Flockhart DA, Drug Interactions: Cytochrome P450 Drug Interaction Table. Indiana University School of Medicine (2007), https://drug-interactions.medicine.iu.edu, accessed May 2021.

In various embodiments, the patient is in further need of treatment with a P-glycoprotein (P-gp) substrate. In some embodiments, the patient is not administered a P-gp substrate in combination with sotorasib. Exemplary P-gp substrates include, but are not limited to dabigatran etexilate, digoxin, fexofenadine, everolimus, cyclosporine, sirolimus, and vincristine. See, e.g., www.fda.gov/drugs/drug-interactions-labeling/drug-development-and-drug-interactions-table-substrates-inhibitors-and-inducers, accessed May 2021. In some embodiments, the patient is not administered a P-gp substrate in combination with sotorasib, wherein the P-gp substrate is a P-gp substrate with a narrow therapeutic window. Exemplary P-gp substrates with a narrow therapeutic window include, but are not limited to, digoxin, everolimus, cyclosporine, sirolimus, and vincristine.

### Patient Characteristics

In various embodiments, the patient has a cancer that was determined to have one or more cells expressing the KRAS^{G12C} mutant protein prior to administration of sotorasib as disclosed herein. Determination of KRAS^{G12C} mutant protein can be assessed as described elsewhere in this disclosure.

In some embodiments, the patient administered the sotorasib in the methods described herein have been previously treated with a different anti-cancer therapy, e.g., at least one—such as one, or two, or three—other systemic cancer therapy. In some embodiments, the patient had previously been treated with one other systemic cancer therapy, such that the sotorasib combination therapy is a second line therapy, e.g., a second line of therapy for treating KRAS^{G12C} metastatic colorectal cancer. In some embodiments, the patient had previously been treated with two other systemic cancer therapies, such that the sotorasib combination therapy as provided herein is a third line therapy, e.g., a third line of therapy for treating KRAS^{G12C} metastatic colorectal cancer. In some embodiments, the patient had not previously been treated with another systemic cancer therapy, such that the sotorasib combination therapy is a first line therapy, e.g., a first line of therapy for treating KRAS^{G12C} metastatic colorectal cancer.

In some embodiments, the prior systemic cancer therapy is a therapy with a KRAS^{G12C} inhibitor. In certain embodiments, the patient exhibits reduced sensitivity to a therapy with a KRAS^{G12C} inhibitor. In some embodiments, the patient is resistant to a therapy with a KRAS^{G12C} inhibitor. In some embodiments, KRAS^{G12C}inhibitor is sotorasib, adagrasib, GDC-6036, D-1553, JDQ443, LY3484356, B11823911, JAB-21822, RMC-6291, or APG-1842. In certain embodiments the KRAS^{G12C} inhibitor is sotorasib. In certain embodiments, the KRAS^{G12C} inhibitor is adagrasib. In some embodiments, the therapy is monotherapy. In some embodiments, the therapy is a therapy comprising the administration of a KRAS^{G12C} inhibitor, for example a combination therapy that comprises the administration of a KRAS^{G12C} inhibitor with a MEK inhibitor or a SHP2 inhibitor (e.g., sotorasib and trametenib, adagrasib and trametinib, sotorasib and RMC-4630, adagrasib and RMC-4630, sotorasib and TNO-155, and adagrasib and TNO-155). In one embodiment, the therapy with a KRAS^{G12C} inhibitor is sotorasib monotherapy. In another embodiment, the therapy with a KRAS^{G12C} inhibitor is monotherapy with adagrasib. In some embodiments, the prior systemic cancer therapy is not a therapy with a KRAS^{G12C} inhibitor. RMC-4630 (CAS No 2172652-48-9, 6-(2-amino-3-chloropyridin-4-yl)sulfanyl-3-[(3S,4S)-4-amino-3-methyl-2-oxa-8-azaspiro[4.5]decan-8-yl]-5-methylpyrazin-2-yl]methanol), is disclosed in International Patent Application Publication No. WO2021/142026, e.g., paragraph [0005]. TNO-155 ((3S,4S)-8-(6-amino-5-((2-amino-3-chloropyridin-4-yl)thio)pyrazin-2-yl)-3-methyl-2-oxa-8-azaspiro[4.5]decan-4-amine), is disclosed in International Patent Application Publication No. WO2021/224867, e.g., paragraph [0014].

As used herein “sensitivity” refers to the way a cancer reacts to a drug, e.g., sotorasib. In exemplary aspects, “sensitivity” means “responsive to treatment” and the concepts of “sensitivity” and “responsiveness” are positively associated in that a cancer or tumor that is responsive to a drug treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistance” and the concept of “resistance” is negatively associated with “sensitivity”. For example, a cancer that is resistant to a drug treatment is either not sensitive nor responsive to that drug or was initially sensitive to the drug and is no longer sensitive upon acquiring resistance; that drug is not or no longer an effective treatment for that tumor or cancer cell.

Prior systemic cancer therapies include, but are not limited to, chemotherapies and immunotherapies. Specific contemplated prior systemic cancer therapies include, but are not limited to, checkpoint inhibitor therapies (e.g., anti-PD1 therapy, anti-PDL1 therapy), platinum based chemotherapy and anti-EGFR therapy. Some examples of anti-PD1 therapy and anti-PDL1 therapies include, but are not limited to, pembrolizumab, nivolumab, cemiplimab, tisielizumab, toripalimab, aspartalizumab, dostarlimab, retifanlimab, simtilimab, pidilizumab atezolizumab, avelumab, durvalumab, and zeluvalimab (AMG 404). Some examples of platinum based chemotherapies include, but are not limited to, carboplatin, oxaliplatin, cisplatin, nedaplatin, satraplatin, lobaplatin, triplatin tetranitrate, picoplatin, ProLindac, and aroplatin. Some examples of anti-EGFR therapy include, but are not limited to, cetuximab and panitumumab.

In some embodiments, the patient has previously been administered a systemic cancer therapy that is a targeted therapy if the cancer was identified to have an actionable oncogenic driver mutation in the epidermal growth factor receptor gene (EGFR), anaplastic lymphoma kinase gene (ALI), and/or ROS proto-oncogene 1 (ROS1). Targeted therapies for EGFR mutations include, but are not limited to, erlotinib, gefitinib, and afatinib. Targeted therapies for ALK mutations include, but are not limited to, crizotinib, entrectinib, lorlatinib, repotrectinib, brigatinib, alkotinib, alectinib, ensartinib, and ceritinib. Targeted therapies for ROS1 mutations include, but are not limited to, crizotinib, entrecetinib, ensartinib, alkotinib, brigatinib, taletrectinib, cabozantinib, repotrectinib, lorlatinib, and ceritinib.

In some embodiments, the patient has not received prior therapy for metastatic disease. In some cases, the patient has not received a prior therapy for the KRAS^{G12C} mutated cancer, e.g., metastatic colorectal cancer and pancreatic cancer. In such cases, the sotorasib therapy as provided herein is a first line therapy.

In some embodiments, the patient has previously received therapy with chemotherapy and an anti-angiogenic agent. In some embodiments, the chemotherapy comprises therapy with fluoropyrimidine, oxaliplatin, and irinotecan. In some embodiments, the anti-angiogenic agent is an anti-VEGF antibody (e.g., bevacizumab and ramucirumab), aflibercept, or regorafenib.

In various embodiments, the patient exhibits an Eastern Cooperative Oncology Group (ECOG) performance status of 0, 1 or 2 (see, e.g., Zubrod et al., 1960). In some embodiments, the patient exhibits an Eastern Cooperative Oncology Group (ECOG) performance status of 0 or 1. Status 0 indicates fully active and able to carry on all pre-disease performance without restriction. Status 1 indicates restricted in physically strenuous activity but ambulatory and able to carry out work of a light or sedentary nature. Status 2 indicates ambulatory and capable of all selfcare but unable to carry out any work activities; up and about more than 50% of waking hours. Status 3 indicates capable of only limited selfcare, confined to bed or chair more than 50% of waking hours. Status 4 indicates completely disabled, cannot carry on any selfcare and totally confined to bed or chair. Status 5 indicates death.

In various embodiments, patient has a cancer that is determined not to be MSI-H. An MSI-H cancer refers to a cancer where the cells have high instability and stands for “microsatellite instability high.” Determination of MSI-H cancers can be assessed by the clinician using well known techniques, e.g., based upon the Bethesda panel-9, or as described, e.g., in U.S. Pat. Nos. 7,521,180; 7,662,595; 10,294,529; or 10,669, 802.

In various cases, the patient has a cancer that is MSI-H. In some cases, the MSI-H cancer is mCRC and the patient has previously been administered a checkpoint inhibitor.

In some cases, the cancer is not MSI-H, e.g., mCRC that is not MSI-H. In various cases, the patient has not received a prior systemic therapy for the KRAS G12C mutation cancer (e.g., mCRC) and the cancer is not MSI-H—i.e., the sotorasib combination therapy is a first line of treatment for the KRAS G12C mutation cancer (e.g., mCRC) that is not MSI-H.

In various cases, the patient has colorectal cancer and the cancer does not comprise a BRAF V600E mutation. Determination of a BRAF V600E mutation can be assessed from a patient sample using an approved mutation test from numerous commercial sources.

### Adverse Events

In some embodiments, the methods comprise administering a reduced total daily dose of sotorasib when the patient experiences an adverse event to the initial total daily dose. For example, in some embodiments, the initial daily dose is 960 mg sotorasib and the reduced total daily dose is 480 mg sotorasib. In some embodiments, the initial daily dose is 480 mg sotorasib and the reduced total daily dose is 240 mg sotorasib. In some embodiments, the methods further comprise administering a second reduced total daily dose of sotorasib when the patient experiences an adverse event to the reduced total daily dose.

The term “adverse event” or “(AE)” as used herein refers to any unfavorable and unintended sign (including an abnormal laboratory finding), symptom, or disease temporally associated with the use of a medical treatment or procedure that may be considered related to the medical treatment or procedure.

In some embodiments, the adverse event is hepatotoxicity (e.g., elevation of liver enzymes), interstitial lung disease (ILD)/pneumonitis, diarrhea, and/or nausea/vomiting.

### Hepatotoxicity

In some embodiments, the adverse event is hepatotoxicity. The term “hepatotoxicity” as used herein refers to a patient having abnormal laboratory values of liver biomarkers (e.g., alkaline phosphatase (ALP), aspartate amino transferase (AST), alanine aminotransferase (ALT), and/or total bilirubin (TBL)), when the patient had baseline levels of the liver biomarker(s) prior to sotorasib administration that were not abnormal laboratory values or were lower than those measured after administration of sotorasib.

Alanine transaminase (ALT), also called serum glutamic pyruvate transaminase (SGPT) or alanine aminotransferase (ALAT), catalyzes the transfer of an amino group from alanine to α-ketoglutarate to produce pyruvate and glutamate. When the liver is damaged, levels of ALT in the blood can rise due to the leaking of ALT into the blood from damaged or necrosed hepatocytes.

Aspartate transaminase (AST) also called serum glutamic oxaloacetic transaminase (SGOT or GOT) or aspartate aminotransferase (ASAT), catalyzes the transfer of an amino group from aspartate to α-ketoglutarate to produce oxaloacetate and glutamate. AST can increase in response to liver damage. Elevated AST also can result from damage to other sources, including red blood cells, cardiac muscle, skeletal muscle, kidney tissue, and brain tissue. The ratio of AST to ALT can be used as a biomarker of liver damage.

Bilirubin is a catabolite of heme that is cleared from the body by the liver. Conjugation of bilirubin to glucuronic acid by hepatocytes produces direct bilirubin, a water-soluble product that is readily cleared from the body. Indirect bilirubin is unconjugated, and the sum of direct and indirect bilirubin constitutes total bilirubin. Elevated total bilirubin can be indicative of liver impairment.

Alkaline phosphatase (ALP) hydrolyzes phosphate groups from various molecules and is present in the cells lining the biliary ducts of the liver. ALP levels in plasma can rise in response to liver damage and are higher in growing children and elderly patients with Paget's disease. However, elevated ALP levels usually reflect biliary tree disease.

In some embodiments, the patient is not suffering from a disorder that results in elevated liver biomarkers. Disorders associated with elevated liver biomarkers (such as AST/ALT and/or TBL values) include, but are not limited to, hepatobiliary tract disease; viral hepatitis (e.g., hepatitis A/B/C/D/E, Epstein-Barr Virus, cytomegalovirus, herpes simplex virus, varicella, toxoplasmosis, and parvovirus); right sided heart failure, hypotension or any cause of hypoxia to the liver causing ischemia; exposure to hepatotoxic agents/drugs or hepatotoxins, including herbal and dietary supplements, plants and mushrooms; heritable disorders causing impaired glucuronidation (e.g., Gilbert's syndrome, Crigler-Najjar syndrome) and drugs that inhibit bilirubin glucuronidation (e.g., indinavir, atazanavir); alpha-one antitrypsin deficiency; alcoholic hepatitis; autoimmune hepatitis; Wilson's disease and hemochromatosis; nonalcoholic fatty liver disease including steatohepatitis; and/or non-hepatic causes (e.g., rhabdomyolysis, hemolysis).

Prior to receiving sotorasib, the baseline liver function of the patient can be assessed by various means known in the art, such as blood chemistry tests measuring biomarkers of liver function. In some embodiments, the methods described herein comprise monitoring liver biomarkers in the patient and withholding sotorasib administration in patients having >Grade 2 abnormal liver function, as assessed by levels of AST and/or ALT. In such embodiments, sotorasib administration is paused until the AST and/or ALT levels in the patient improve(s) to Grade 1 or better (baseline).

Adverse effect Grades for abnormal liver function are defined herein by the modified Common Toxicity Criteria (CTC) provided in Table 1. See the National Cancer Institute Common Terminology Criteria for Adverse Events v5.0 (NCI CTCAE) published Nov. 27, 2017 by the National Cancer Institute, incorporated herein by reference in its entirety.

TABLE 1
Common Toxicity Criteria
Toxicity grades

| Toxicity | 0 | 1 | 2 | 3 | 4 |
|---|---|---|---|---|---|
| ALT | WNL | >ULN − 3.0 × ULN, | >3-5 × ULN, if | >5-20 × ULN, | >20 × ULN if |
| | | if baseline was | baseline was | if baseline was | baseline was |
| | | normal; 1.5-3.0 × | normal, >3.0-5.0 × | normal; >5.0-20.0 × | normal; >20 × |
| | | baseline if baseline | baseline if | baseline | baseline if |
| | | was abnormal | baseline was | if baseline was | baseline was |
| | | | abnormal | abnormal | abnormal |
| AST | WNL | >ULN − 3.0 × ULN | >3-5 × ULN if | >5-20 × ULN | >20 × ULN if |
| | | if baseline was | baseline was | if baseline was | baseline was |
| | | normal; 1.5-3.0 × | normal, >3.0-5.0 × | normal; >5.0-20.0 × | normal; >20 × |
| | | baseline if baseline | baseline | baseline | baseline if |
| | | was abnormal | id baseline was | if baseline was | baseline was |
| | | | abnormal | abnormal | abnormal |
| Bilirubin | WNL | >ULN − 1.5 × ULN | >1.5-3 × ULN if | >3-10 × ULN | >10 × ULN if |
| | | if baseline was | baseline was | if baseline was | baseline was |
| | | normal; >1.0-1.5 × | normal; >1.5-3.0 × | normal; >3.0-10 × | normal; >10.0 × |
| | | baseline if baseline | baseline if | baseline if | baseline if |
| | | was abnormal | baseline was | baseline was | baseline was |
| | | | abnormal | abnormal | abnormal |
| ALP | WNL | > ULN − 2.5 × ULN | >2.5-5.0 × | >5-20 × ULN | >20 × ULN |
| | | if baseline was | ULN if baseline | if baseline was | if baseline was |
| | | normal; 2.0-2.5 × | was normal, | normal; >5.0-20.0 × | normal; >20 × |
| | | baseline if baseline | >2.5-5.0 × | baseline if | baseline if |
| | | was abnormal | baseline if | baseline was | baseline was |
| | | | baseline was | abnormal | abnormal |
| | | | abnormal | | |

ALP = alkaline phosphatase;
ALT = alanine aminotransferase;
AST = aspartate aminotransferase;
ULN = upper limit of normal;
WNL = within normal limits

Grade 0 levels are characterized by biomarker levels within normal limits (WNL). “Normal” liver function, as used herein, refers to Grade 0 adverse effects. “Abnormal” liver function, as used herein, refers to Grade 1 and above adverse effects.

“Grade 1 liver function abnormalities” include elevations in ALT or AST greater than the ULN and less than or equal to 3-times the ULN if baseline was normal; 1.5-3.0×baseline if baseline was abnormal. Grade 1 liver function abnormalities also include elevations of bilirubin levels greater than the ULN and less than or equal to 1.5-times the ULN if baseline was normal; >1.0-1.5×baseline if baseline was abnormal. Grade 1 liver function abnormalities also include elevations of ALP greater than the ULN and less than or equal to 2.5-times the ULN if baseline was normal; >2.0-2.5×baseline if baseline was abnormal.

“Grade 2 liver function abnormalities” include elevations in ALT or AST greater than 3-times and less than or equal to 5-times the upper limit of normal (ULN) if baseline was normal; >3.0-5.0×baseline if baseline was abnormal. Grade 2 liver function abnormalities also include elevations of bilirubin levels greater than 1.5-times and less than or equal to 3-times the ULN if baseline was normal; >1.5-3.0×baseline if baseline was abnormal. Grade 2 liver function abnormalities also include elevations of ALP greater than 2.5-times and less than or equal to 5-times the ULN if baseline was normal; >2.5-5.0×baseline if baseline was abnormal.

“Grade 3 liver function abnormalities” include elevations in ALT, AST, or ALP greater than 5-times and less than or equal to 20-times the ULN if baseline was normal; >5.0-20.0×baseline if baseline was abnormal. Grade 3 liver function abnormalities also include elevations of bilirubin levels greater than 3-times and less than or equal to 10-times the ULN if baseline was normal; >3.0-10×baseline if baseline was abnormal.

“Grade 4 liver function abnormalities” include elevations in ALT, AST, or ALP greater than 20-times the ULN if baseline was normal; >20×baseline if baseline was abnormal. Grade 4 liver function abnormalities also include elevations of bilirubin levels greater than 10 times the ULN if baseline was normal; >10.0×baseline if baseline was abnormal.

The ULN for various indicators of liver function depends on the assay used, the patient population, and each laboratory's normal range of values for the specified biomarker, but can readily be determined by the skilled practitioner. Exemplary values for normal ranges for a healthy adult population are set forth in Table 2 below. See Cecil Textbook of Medicine, pp. 2317-2341, W.B. Saunders & Co. (1985).

TABLE 2
Upper Limit of Normal (ULN) Values

| | | |
|---|---|---|
| ALT | 8-20 | U/L |
| AST | 8-20 | u/l |
| Bilirubin | 0.2-1 | mg/dL |
| | 3.4-17.1 | μmol/L |
| ALP | 20-70 | U/L |

In any of the methods described herein, the total daily dose of sotorasib is reduced (e.g., from 960 mg to 480 mg, or from 480 mg to 240 mg) when the AST and/or ALT level(s) in the patient is/are elevated, e.g., to a Grade 2 or Grade 3 level, where the baseline AST and/or ALT levels of the patient were below Grade 2 or Grade 3 levels. In some embodiments, the total daily dose of sotorasib is reduced (e.g., from 960 mg to 480 mg, or from 480 mg to 240 mg), when the AST and/or ALT level(s) in the patient is/are elevated to a Grade 1 level, wherein the baseline AST and/or ALT levels of the patient were below Grade 1 levels.

Alternatively, in any of the methods disclosed herein, the total daily dose of sotorasib is reduced (e.g., from 960 mg to 480 mg, or from 480 mg to 240 mg) when (1) AST and bilirubin levels in the patient are elevated, or (2) when AST or ALP levels in the patient are elevated, or (3) when ALT and bilirubin levels in the patient are elevated, or (4) when ALT and ALP levels in the patient are elevated, or (5) when bilirubin and ALP levels in the patient are elevated, e.g., to a Grade 1, Grade 2, Grade 3 or Grade 4 level, wherein the baseline AST, bilirubin, ALP, and/or ALT levels of the patient were below Grade 1, Grade 2, Grade 3 or Grade 4 levels, respectively. Alternatively, in any of the methods disclosed herein, three biomarkers of liver function may be elevated in the patient (e.g., ALT and AST and bilirubin, or ALT and AST and ALP) to a Grade 1, Grade 2, Grade 3 or Grade 4 level, wherein the baseline biomarker levels of the patient were below Grade 1, Grade 2, Grade 3 or Grade 4 levels, respectively.

In some embodiments, the total daily dose of sotorasib is reduced (e.g., from 960 mg to 480 mg, or from 480 mg to 240 mg) when the level of ALT and/or AST is greater than about 3 times compared to the upper limit of normal (ULN). In a related embodiment, the abnormal level of ALT and/or AST is greater than about 3- to about 5-fold increase compared to the upper limit of normal (ULN), i.e., a “Grade 2 abnormality”. In some embodiments, where the patient has an abnormal baseline, the Grade 2 abnormality is an abnormal level of ALT and/or AST greater than about 3-fold to about 5-fold increase compared to baseline. In some embodiments, the abnormal level of ALP is greater than about 2.5- to about 5-fold increase compared to the upper limit of normal (ULN), i.e., a “Grade 2 abnormality”. In some embodiments, where the patient has an abnormal baseline, the Grade 2 abnormality is an abnormal level of ALP greater than about 2.5-fold to about 5-fold increase compared to baseline. In some embodiments, the abnormal level of bilirubin is greater than about 1.5- to about 3-fold increase compared to the upper limit of normal (ULN), i.e., a “Grade 2 abnormality”. In some embodiments, where the patient has an abnormal baseline, the Grade 2 abnormality is an abnormal level of bilirubin greater than about 1.5-fold to about 3-fold increase compared to baseline.

In some embodiments, the total daily dose of sotorasib is reduced (e.g., from 960 mg to 480 mg, or from 480 mg to 240 mg) when the level of ALT and/or AST is greater than about 5 times compared to the upper limit of normal (ULN). In some embodiments, the total daily dose is reduced when the level of ALT, AST, or ALP is greater than about 5- to about 20-fold increase compared to the upper limit of normal (ULN), i.e., a “Grade 3 abnormality”. In some embodiments, where the patient has an abnormal baseline, the Grade 3 abnormality is an abnormal level of ALT and/or AST greater than about 5-fold to about 20-fold increase compared to baseline. In some embodiments, the abnormal level of ALP is greater than about 5- to about 20-fold increase compared to the upper limit of normal (ULN), i.e., a “Grade 3 abnormality”. In some embodiments, where the patient has an abnormal baseline, the Grade 3 abnormality is an abnormal level of ALP greater than about 5-fold to about 20-fold increase compared to baseline. In some embodiments, the total daily dose is reduced when the level of bilirubin is greater than about 3- to about 10-fold increase compared to the upper limit of normal (ULN), i.e., a “Grade 3 abnormality”. In some embodiments, where the patient has an abnormal baseline, the Grade 3 abnormality is an abnormal level of bilirubin greater than about 3-fold to about 10-fold increase compared to baseline.

In some embodiments, the total daily dose of sotorasib is reduced (e.g., from 960 mg to 480 mg, or from 480 mg to 240 mg) when the level of ALT and/or AST is greater than about 20 times compared to the upper limit of normal (ULN) (i.e., a “Grade 4 abnormality”). In some embodiments, where the patient has an abnormal baseline, the Grade 4 abnormality is an abnormal level of ALT and/or AST greater than about 20-fold increase compared to baseline. In some embodiments, the abnormal level of ALP is greater than about 20-fold increase compared to the upper limit of normal (ULN), i.e., a “Grade 4 abnormality”. In some embodiments, where the patient has an abnormal baseline, the Grade 4 abnormality is an abnormal level of ALP greater than about 20-fold increase compared to baseline. In some embodiments, the total daily dose is reduced when the level of bilirubin is greater than about 10-fold increase compared to the upper limit of normal (ULN), i.e., a “Grade 4 abnormality”. In some embodiments, where the patient has an abnormal baseline, the Grade 4 abnormality is an abnormal level of bilirubin greater than about 10-fold increase compared to baseline.

In some embodiments, the methods described herein further comprise increasing the total dose of sotorasib (e.g., from 240 mg to 480 mg, or from 480 mg to 960 mg) when liver biomarker(s) in the patient has improved to a Grade 1 or better (e.g., baseline).

### Nausea/Vomiting

In some embodiments, the adverse event is nausea or vomiting. In some embodiments, the nausea/vomiting is present despite appropriate supportive care (e.g., anti-emetic therapy). “Nausea” as used herein refers to a disorder characterized by a queasy sensation and/or the urge to vomit.

Adverse effect Grades for nausea and vomiting are defined herein by the modified Common Toxicity Criteria (CTC) provided in Table 3. See the National Cancer Institute Common Terminology Criteria for Adverse Events v5.0 (NCI CTCAE) published Nov. 27, 2017 by the National Cancer Institute, incorporated herein by reference in its entirety.

TABLE 3

| | Grade 1 | Grade 2 | Grade 3 | Grade 4 |
|---|---|---|---|---|
| Nausea | Loss of appetite | Oral intake | Inadequate oral | — |
| | without alteration | decreased without | caloric or fluid | |
| | in eating habits | significant weight | intake; tube | |
| | | loss, dehydration | feeding, total | |
| | | or malnutrition | parenteral nutrition | |
| | | | (TPN), or | |
| | | | hospitalization | |
| | | | indicated | |
| Vomiting | Intervention | Outpatient IV | Tube feeding, | Life- |
| | not indicated | hydration; medical | TPN, or | threatening |
| | | intervention | hospitalization | consequences |
| | | indicated | indicated | |

In some embodiments, the methods described herein comprise withholding sotorasib administration in a patient having ≥Grade 3 nausea until the patient has improved to ≤Grade 1 or baseline. In some embodiments, once the patient has improved to ≤Grade 1 or baseline, the methods comprise administering a reduced total daily dose of sotorasib (e.g., from 960 mg to 480 mg, or from 480 mg to 240 mg) to the patient.

In some embodiments, the methods described herein comprise withholding sotorasib administration in a patient having ≥Grade 3 vomiting until the vomiting improves to ≤Grade 1 or baseline. In some embodiments, once the patient has improved to ≤Grade 1 or baseline, the methods comprise administering a reduced total daily dose of sotorasib (e.g., from 960 mg to 480 mg, or from 480 mg to 240 mg) to the patient.

In some embodiments, the methods described herein further comprise increasing the total dose of sotorasib (e.g., from 240 mg to 480 mg, or from 480 mg to 960 mg) when nausea in the patient has improved to a Grade 1 or better (e.g., baseline).

### Diarrhea

In some embodiments, the adverse event is diarrhea. In some embodiments, the diarrhea is present despite appropriate supportive care (e.g., anti-diarrheal therapy).

Adverse effect Grades for diarrhea are defined herein by the modified Common Toxicity Criteria (CTC) provided in Table 4. See the National Cancer Institute Common Terminology Criteria for Adverse Events v5.0 (NCI CTCAE) published Nov. 27, 2017 by the National Cancer Institute, incorporated herein by reference in its entirety.

TABLE 4

| | Grade 1 | Grade 2 | Grade 3 | Grade 4 |
|---|---|---|---|---|
| Diarrhea | Increase of <4 | Increase of 4-6 | Increase of ≥7 | Life-threatening |
| | stools per day over | stools per day over | stools per day over | consequences; |
| | baseline; mild | baseline; moderate | baseline; | urgent |
| | increase in ostomy | increase in ostomy | hospitalization | intervention |
| | output compared to | output compared to | indicated; severe | indicated |
| | baseline | baseline; limiting | increase in ostomy | |
| | | instrumental | output compared to | |
| | | activities of daily | baseline; limiting | |
| | | living (ADL) | self care ADL | |

In some embodiments, the methods described herein comprise withholding sotorasib administration in a patient having ≥Grade 3 diarrhea until the patient has improved to ≤Grade 1 or baseline. In some embodiments, once the patient has improved to ≤Grade 1 or baseline, the methods comprise administering a reduced total daily dose of sotorasib (e.g., from 960 mg to 480 mg, or from 480 mg to 240 mg) to the patient.

In some embodiments, the methods described herein further comprise increasing the total dose of sotorasib (e.g., from 240 mg to 480 mg, or from 480 mg to 960 mg) when diarrhea in the patient has improved to a Grade 1 or better (e.g., baseline).

### Interstitial Lung Disease

In some embodiments, the adverse event is interstitial lung disease (ILD) or pneumonitis. In cases where ILD or pneumonitis is suspected at any grade level, sotorasib is withheld. In cases where ILD or pneumonitis is confirmed, and no other causes of the ILD or pneumonitis is identified, sotorasib is permanently discontinued.

### Response to Sotorasib Combination Therapy

Response rates or results for patients administered sotorasib in the methods disclosed herein can be measured in a number of ways, after the patient has been taking sotorasib for a suitable length of time. In various embodiments, a patient is administered sotorasib for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, or 24 months. In various embodiments, the patient is administered sotorasib for at least 1 month. In various embodiments, the patient is administered sotorasib for at least 3 months. In various embodiments, the patient is administered sotorasib for at least 6 months.

The patient can respond to the sotorasib combination therapy as measured by at least a stable disease (SD), as determined by Response Evaluation Criteria in Solid Tumors (RECIST) 1.1 protocol (Eisenhauer, et al., 2009). An at least stable disease is one that is a stable disease, has shown a partial response (PR) or has shown a complete response (CR) (i.e., “at least SD”=SD+PR+CR, often referred to as disease control). In various embodiments, the stable disease is neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Progression free survival (PFS) can be assessed as described in the RECIST 1.1 protocol. In various embodiments, the patient exhibits a PFS of at least 1 month. In various embodiments, the patient exhibits a PFS of at least 3 months. In some embodiments, the patient exhibits a PFS of at least 6 months.

Additional means for assessing response are described in detail in the examples below and can generally be applied to the methods disclosed herein.

### KRAS G12C Cancers

Without wishing to be bound by any particular theory, the following is noted: sotorasib is a small molecule that specifically and irreversibly inhibits KRAS^{G12C} (Hong et al., 2020). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Canon et al., 2019, and Lanman et al., 2020).

Sotorasib was evaluated in a Phase 1 dose escalation and expansion trial with 129 patients having histologically confirmed, locally advanced or metastatic cancer with the KRAS G12C mutation identified by local molecular testing on tumor tissues, including 59 patients with non-small cell lung cancer, 42 patients with colorectal cancer, and 28 patients with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6)).

KRAS G12C mutations occur with the alteration frequencies shown in the table below (Cerami et al., 2012; Gao et al., 2013). For example, the table shows that 11.6% of patients with non-small cell lung cancer have a cancer, wherein one or more cells express KRAS G12C mutant protein. Accordingly, sotorasib, which specifically and irreversibly bind to KRAS^{G12C} is useful for treatment of patients having a cancer, including, but not limited to the cancers listed in Table 5 below.

TABLE 5

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal | 0.4 |
| Neuroendocrine Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma. In various embodiments, the cancer is non-small cell lung cancer, and in some specific embodiments, metastatic or locally advanced non-small cell lung cancer. In various embodiments, the cancer is colorectal cancer. In some embodiments, the cancer is pancreatic cancer.

Methods of Detecting KRAS, STK11, KEAP1, EGFR, ALK, and/or ROS1 Mutation Status

The presence or absence of G12C, STK11, KEAP1, EGFR, ALK and/or ROS1 mutations in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequence of wild-type human KRAS (nucleotide sequence set forth in Genbank Accession No. BC010502; amino acid sequence set forth in Genbank Accession No. AG009594), STK11 (Gene ID: 6794; available at www.ncbi.nlm.nih.gov/gene/6794; accessed January 2020), KEAP1 (Gene ID: 9817; available at www.ncbi.nlm.nih.gov/gene/9817; accessed January 2020), EGFR (Gene ID: 1956; available at www.ncbi.nlm.nih.gov/gene/1956; accessed March 2021), ALK(Gene ID: 238; available at www.ncbi.nlm.nih.gov/gene/238; accessed March 2021), and ROS1 (Gene ID: 6098; available at www.ncbi.nlm.nih.gov/gene/6098; accessed March 2021) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TaqMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations present in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations present in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations present in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; Alizadeh et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art.

Methods for determining whether a tumor or cancer comprises a mutation can use a variety of samples. In some embodiments, the sample is taken from a patient having a tumor or cancer. In some embodiments, the sample is a fresh tumor/cancer sample. In some embodiments, the sample is a frozen tumor/cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRAS G12C mutated cancer, or whether the tumor or tissue sample obtained from such patient contains cells with a mutation. In some embodiments, the test for a KRAS mutation used is Therascreen® KRAS RGQ PCR Kit (Qiagen). The Therascreen® KRAS RGQ PCR Kit is a real-time qualitative PCR assay for the detection of 7 somatic mutations in codons 12 and 13 of the human KRAS oncogene (G12A, G12D, G12R, G12C, G12S, G12V, and G13D) using the Rotor-Gene Q MDx 5plex HRM instrument. The kit is intended for use with DNA extracted from FFPE samples of NSCLC or CRC acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

In some embodiments, the cancer has been identified as having a KRAS G12C mutation. In some embodiments, the cancer has been identified as having a mutation of STK11, e.g., a loss-of-function mutation. In some embodiments, the cancer has been identified as having a mutation of KEAP1, e.g., a loss-of-function mutation. In some embodiments, the cancer has been identified as having wild-type STK11. In some embodiments, the cancer has been identified as having wild-type KEAP1.

In various embodiments, the cancer has been identified as having a loss-of-function mutation of STK11 and wild-type KEAP1. In some embodiments, the cancer has been identified as having a loss-of-function mutation of STK11 and a loss-of-function mutation of KEAP1. In some embodiments, the cancer has been identified as having wild-type of STK11 and wild-type KEAP1. In some embodiments, the cancer has been identified as having wild-type of STK11 and a loss-of-function mutation of KEAP1.

The term “loss-of-function mutation” as used herein refers to a mutation (e.g., a substitution, deletion, truncation, or frameshift mutation) that results in expression of a mutant protein that no longer exhibits wild-type activity (e.g., reduced or eliminated wild-type biological activity or enzymatic activity), results in expression of only a fragment of the protein that no longer exhibits wild-type activity, or results in no expression of the wild-type protein. For example, a loss-of-function mutation affecting the STK11 gene in a cell may result in the loss of expression of the STK11 protein, expression of only a fragment of the STK11 protein, or expression of the STK11 protein that exhibits diminished or no enzymatic activity (e.g., no serine/threonine kinase enzymatic activity) in the cancerous cell. Similarly, a loss-of-function mutation affecting the KEAP1 gene in a cell may result in the loss of expression of the KEAP1 protein, expression of only a fragment of the KEAP1 protein, or expression of a KEAP1 protein that exhibits diminished or no activity (e.g., inability to interact with or activate Nuclear factor erythroid 2-related factor 2 (NRF2)) in the cell.

### Methods of Detecting PD-L1 Protein Expression

PD-L1 expression can be determined by methods known in the art. For example, PD-L1 expression can be detected using PD-L1 IHC 22C3 pharmDx, an FDA-approved in vitro diagnostic immunohistochemistry (IHC) test developed by Dako and Merck as a companion test for treatment with pembrolizumab. This is a qualitative assay using Monoclonal Mouse Anti-PD-L1, Clone 22C3 PD-L1 and EnVision FLEX visualization system on Autostainer Lin 48 to detect PD-L1 in FFPE samples, such as human non-small cell lung cancer tissue. Expression levels can be measured using the tumor proportion score (TPS), which measures the percentage of viable tumor cells showing partial or complete membrane staining at any intensity. Staining can show PD-L1 expression from 0% to 100%.

PD-L1 expression can also be detected using PD-L1 IHC 28-8 pharmDx, the FDA-approved in vitro diagnostic immunohistochemistry (IHC) test developed by Dako and Bristol-Myers Squibb as a companion test for treatment with nivolumab. This qualitative assay uses the Monoclonal rabbit anti-PD-L1, Clone 28-8 and EnVision FLEX visualization system on Autostainer Lin 48 to detect PD-L1 in formalin-fixed, paraffin-embedded (FFPE) human cancer tissue.

Other commercially available tests for PD-L1 detection include the Ventana SP263 assay (developed by Ventana in collaboration with AstraZeneca) that utilizes monoclonal rabbit anti-PD-L1, Clone SP263 and the Ventana SP142 Assay (developed by Ventana in collaboration with Genentech/Roche) that uses rabbit monoclonal anti-PD-L1 clone SP142.

In some embodiments, a test approved by a regulatory authority, such as the US Food and Drug Administration (FDA), is used to determine the PD-L1 TPS of a cancer as disclosed herein. In various embodiment, the PD-L1 TPS is determined using an immunohistochemistry (IHC) test. In some embodiments, the IHC test is the PD-L1 IHC 22C3 pharmDx test. In various embodiments, the IHC test conducted with samples acquired by, for example, resection, CNB, or FNA.

In various embodiment, the patient has a PD-L1 TPS of less than 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than 50%, or less than 1%. In various embodiments, the patient has a PD-L1 TPS of more than or equal to 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than or equal to 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS of less than or equal to 50%, or less than or equal to 1%. In various embodiments, the patient has a PD-L1 TPS of more than 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PD-L1 TPS score a range bound by any of the values cited in the foregoing embodiments. For example, the patient has a PD-L1 TPS score in the range of less than 50% and more than or equal to 1%, less than or equal to 50% and more than 1%, less than or equal to 50% and more than or equal to 1%, or less than 50% and more than 1%.

In various embodiments, the patient has a PD-L1 TPS score in the range of less than 50% and more than or equal to 1%. In some embodiments, the patient has a PD-L1 TPS score in the range of more than or equal to 0% and less than 1%. In some embodiments, the patient has a PD-L1 TPS score in the range of more than 50% and less than or equal to 100%. In some embodiments, the patient has a PD-L1 TPS score of less than 1%. In some embodiments, the patient as a PD-L1 TPS score of 1-49%. In some embodiments, the patient has a PD-L1 TPS score of 50% or greater (i.e., 50%-100%).

### EMBODIMENTS

1. A method of treating cancer comprising a KRAS G12C mutation in a patient comprising administering to the patient sotorasib and an anti-epidermal growth factor receptor (EGFR) antibody in amounts effective to treat the cancer.

2. The method of embodiment 1, comprising administering 960 mg sotorasib to the patient daily.

3. The method of embodiment 1, comprising administering 720 mg sotorasib to the patient daily.

4. The method of embodiment 1, comprising administering 480 mg sotorasib to the patient daily.

5. The method of embodiment 1, comprising administering 240 mg sotorasib to the patient daily.

6. The method of any one of embodiments 1-5, comprising administering sotorasib to the patient once daily.

7. The method of any one of embodiments 1-5, comprising administering sotorasib to the patient twice daily.

8. The method of any one of embodiments 1-5, comprising administering the anti-epidermal growth factor receptor (EGFR) antibody to the patient every two weeks.

9. The method of embodiment 8, wherein the anti-EGFR antibody comprises the heavy chain HCDR1 of SEQ ID NO: 1, HCDR2 of SEQ ID NO: 2, HCDR3 of SEQ ID NO: 3, light chain LCDR1 of SEQ ID NO: 6, LCDR2 of SEQ ID NO: 7, and LCDR3 of SEQ ID NO: 8.

10. The method of embodiment 9, wherein the anti-EGFR antibody comprises the heavy chain variable region sequence of SEQ ID NO: 4 and the light chain variable region of SEQ ID NO: 9.

11. The method of embodiment 10, wherein the anti-EGFR antibody comprises the heavy chain sequence of SEQ ID NO: 5 and the light chain sequence of SEQ ID NO: 10.

12. The method of embodiment 8, wherein the anti-EGFR antibody is panitumumab.

13. The method of any one of embodiments 1-12, comprising administering 6 mg/kg panitumumab to the patient.

14. The method of any one of embodiments 1-12, comprising administering 3 mg/kg panitumumab to the patient.

15. The method of any one of embodiments 1-12, comprising administering to the patient
(a) 960 mg sotorasib daily; and
(b) 6 mg/kg panitumumab via IV administration every two weeks.

16. The method of any one of embodiments 1-12, comprising administering to the patient
(a) 720 mg sotorasib daily; and
(b) 6 mg/kg panitumumab via IV administration every two weeks.

17. The method of any one of embodiments 1-12, comprising administering to the patient
(a) 480 mg sotorasib daily; and
(b) 6 mg/kg panitumumab via IV administration every two weeks.

18. The method of any one of embodiments 1-12, comprising administering to the patient
(a) 960 mg sotorasib daily; and
(b) 3 mg/kg panitumumab via IV administration every two weeks.

19. The method of any one of embodiments 1-18, further comprising administering irinotecan, 5-FU, and leucovorin to the patient.

20. The method of embodiment 19, comprising administering 400 mg/m² leucovorin via IV administration to the patient.

21. The method of any one of embodiments 1-18, further comprising administering irinotecan, 5-FU, and levoleucovorin to the patient.

22. The method of embodiment 21, comprising administering 200 mg/m² levoleucovorin via IV administration to the patient.

23. The method of any one of embodiments 1-22, comprising administering 180 mg/m² irinotecan via IV administration to the patient.

24. The method of any one of embodiments 1-22, comprising administering 400 mg/m² 5-FU via IV administration to the patient.

25. The method of any one of embodiments 1-20, and 23-24, comprising administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

26. The method of any one of embodiments 1-18, and 21-24, comprising administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

27. The method of any one of embodiments 1-18, further comprising administering irinotecan and 5-FU to the patient.

28. The method of embodiment 27, comprising administering 180 mg/m² irinotecan via IV administration to the patient.

29. The method of embodiment 27, comprising administering 150 mg/m² irinotecan via IV administration to the patient.

30. The method of any one of embodiments 1-18 and 28, comprising administering via IV administration 180 mg/m² irinotecan, and 400 mg/m² 5-FU IV bolus and 2400 mg/m² 5-FU IV continuous infusion over 46-48 hours to the patient every two weeks.

31. The method of any one of embodiments 1-18 and 27, comprising administering via IV administration 150 mg/m² irinotecan, and 400 mg/m² 5-FU IV bolus and 2400 mg/m² 5-FU IV continuous infusion over 46-48 hours to the patient every two weeks.

32. The method of any one of embodiments 1-31, wherein the cancer is a solid tumor.

33. The method of any one of embodiments 1-32, wherein the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma.

34. The method of any one of embodiments 1-33, wherein the cancer is non-small cell lung carcinoma (NSCLC) or colorectal cancer (CRC).

35. The method of any one of embodiments 1-33, wherein the cancer is non-small cell lung carcinoma (NSCLC).

36. The method of any one of embodiments 1-33, wherein the cancer is metastatic pancreatic cancer.

37. The method of any one of embodiments 1-36, wherein the patient has previously received at least one other systemic cancer therapy.

38. The method of embodiment 37, wherein at least one systemic cancer therapy is selected from therapy with a KRAS^{G12C} inhibitor, anti-PD-1 therapy, anti-PD-L1 therapy, and platinum-based chemotherapy.

39. The method of embodiments 37, wherein the at least one systemic cancer therapy is not a therapy with a KRAS^{G12C} inhibitor.

40. The method of any one or embodiments 1-39, wherein the cancer is colorectal cancer (CRC).

41. The method of any one of embodiments 1-34 and 39, wherein the patient has previously received therapy with fluoropyrimidine, oxaliplatin, irinotecan, and an anti-angiogenic agent.

42. The method of embodiment 40 or 41, wherein the patient has previously received treatment with a checkpoint inhibitor.

43. The method of any one of embodiments 1-34 and 40-42, wherein the patient is resistant to therapy with a KRAS^{G12C} inhibitor.

44. The method of any one of embodiments 1-34, 36, and 40-43, wherein the patient has previously received at least one other therapy for metastatic disease.

45. The method of any one of embodiments 1-34, 36, and 40-43, wherein the patient has previously received one other therapy for metastatic disease.

46. The method of any one of embodiments 1-34 and 40, wherein the patient has not received prior therapy for metastatic disease.

47. The method of any one of embodiments 1-46, wherein the patient does not have an active brain metastases or leptomeningeal disease from a non-brain tumor.

48. The method of any one of embodiments 1-47, wherein the patient did not have a myocardial infarction 6 months before beginning treatment.

49. The method of any one of embodiments 1-48, wherein the patient exhibits at least a stable disease (SD) after 1, 3, or 6 months of sotorasib and panitumumab therapy, as measured by RECIST 1.1 protocol.

50. The method of any one of embodiments 1-49, wherein the patient exhibits at least a partial response (PR) after 1, 3, or 6 months of sotorasib and panitumumab therapy, as measured by RECIST 1.1 protocol.

51. The method of any one of embodiments 1-50, wherein the patient exhibits a progression free survival (PFS) of at least 3 months.

52. The method of any one of embodiments 1-51, wherein the patient is not suffering from a hepatitis A infection, a hepatitis B infection, or a hepatitis C infection.

53. The method of any one of embodiments 1-52, wherein the patient is not suffering from interstitial pneumonitis or pulmonary fibrosis.

54. The method of any one of embodiments 1-53, wherein the patient is in further need of treatment with an acid-reducing agent.

55. The method of embodiment 54, wherein the acid-reducing agent is a proton pump inhibitor (PPI), a H2 receptor antagonist (H2RA), or a locally acting antacid.

56. The method of embodiment 54 or embodiment 55, wherein the acid-reducing agent is a locally acting antacid, and wherein sotorasib is administered about 4 hours before or about 10 hours after the locally acting antacid.

57. The method of embodiment 55 or embodiment 56, wherein the locally acting antacid is sodium bicarbonate, calcium carbonate, aluminum hydroxide, or magnesium hydroxide.

58. The method of any one of embodiments 1-57, wherein the patient is in further need of treatment with a proton pump inhibitor (PPI) or H2 receptor antagonist (H2RA).

59. The method of embodiment 58, wherein the patient is not administered a PPI or a H2RA in combination with sotorasib.

60. The method of any one of embodiments 55, 58, or 59, wherein the PPI is omeprazole, pantoprazole, esomeprazole, lansoprazole, rabeprazole, or dexlansoprazole.

61. The method of any one of embodiments 55, 58, or 59, wherein the H2RA is famotidine, ranitidine, cimetidine, nizatidine, roxatidine, or lafutidine.

62. The method of any one of embodiments 1-61, wherein the patient is in further need of treatment with a CYP3A4 inducer.

63. The method of embodiment 62, wherein the patient is not administered a CYP3A4 inducer in combination with sotorasib.

64. The method of embodiment 62 or 63, wherein the CYP3A4 inducer is a barbiturate, brigatinib, carbamazepine, clobazam, dabrafenib, efavirenz, elagolix, enzalutamide, eslicarbazepine, glucocorticoid, letermovir, lorlatinib, modafinil, nevirapine, oritavancin, oxcarbazepine, perampanel, phenobarbital, phenytoin, pioglitazone, rifabutin, rifampin, telotristat, or troglitazone.

65. The method of embodiment 62 or embodiment 63, wherein the patient is not administered a strong CYP3A4 inducer in combination with sotorasib.

66. The method of embodiment 65, wherein the strong CYP3A4 inducer is phenytoin or rifampin.

67. The method of any one of embodiments 1-66, wherein the patient is in further need of treatment with a CYP3A4 substrate.

68. The method of embodiment 67, wherein the patient is not administered a CYP3A4 substrate in combination with sotorasib.

69. The method of embodiment 67 or 68, wherein the CYP3A4 substrate is abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus(fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, or ziprasidone.

70. The method of any one of embodiments 1-69, wherein the patient is in further need of treatment with a P-glycoprotein (P-gp) substrate.

71. The method of embodiment 70, wherein the patient is not administered a P-gp substrate in combination with sotorasib.

72. The method of embodiment 70 or embodiment 71, wherein the P-gp substrate is etexilate, digoxin, or fexofenadine.

73. The method of any one of embodiments 1-72, wherein the cancer exhibits a PD-L1 tumor proportion score (TPS) of 1-49%.

74. The method of any one of embodiments 1-73, wherein the cancer exhibits a PD-L1 tumor proportion score (TPS) of less than 1%.

75. The method of any one of embodiments 1-74, wherein the cancer exhibits a PD-L1 tumor proportion score (TPS) of 50-100%.

76. The method of any one of embodiments 1-75, wherein the cancer further comprises a STK11 mutation.

77. The method of any one of embodiments 1-76, wherein the cancer further comprises a KEAP1 mutation.

78. The method of embodiment 76 or embodiment 77, wherein the mutation is a loss-of-function mutation.

### First Set of Alternative Embodiments

1. A method of treating cancer comprising a KRAS G12C mutation in a patient comprising administering to the patient sotorasib and an anti-epidermal growth factor receptor (EGFR) antibody in amounts effective to treat the cancer.

2. The method of embodiment 1, comprising administering 960 mg sotorasib to the patient daily.

3. The method of embodiment 1, comprising administering 240 mg sotorasib to the patient daily.

4. The method of any one of embodiments 1-3, comprising administering sotorasib to the patient once daily.

5. The method of any one of embodiments 1-3, comprising administering sotorasib to the patient twice daily.

6. The method of any one of embodiments 1-5, comprising administering the anti-epidermal growth factor receptor (EGFR) antibody to the patient every two weeks.

7. The method of embodiment 6, wherein the anti-EGFR antibody comprises the heavy chain HCDR1 of SEQ ID NO: 1, HCDR2 of SEQ ID NO: 2, HCDR3 of SEQ ID NO: 3, light chain LCDR1 of SEQ ID NO: 6, LCDR2 of SEQ ID NO: 7, and LCDR3 of SEQ ID NO: 8.

8. The method of embodiment 7, wherein the anti-EGFR antibody comprises the heavy chain variable region sequence of SEQ ID NO: 4 and the light chain variable region of SEQ ID NO: 9.

9. The method of embodiment 7, wherein the anti-EGFR antibody comprises the heavy chain sequence of SEQ ID NO: 5 and the light chain sequence of SEQ ID NO: 10.

10. The method of embodiment 7, wherein the anti-EGFR antibody is panitumumab.

11. The method of any one of embodiments 1-10, comprising administering 6 mg/kg panitumumab to the patient.

12. The method of any one of embodiments 1-10, comprising administering to the patient
(a) 960 mg sotorasib daily; and
(b) 6 mg/kg panitumumab via IV administration every two weeks.

13. The method of any one of embodiments 1-10, comprising administering to the patient
(a) 240 mg sotorasib daily; and
(b) 6 mg/kg panitumumab via IV administration every two weeks.

14. The method of any one of embodiments 1-13, further comprising administering irinotecan, 5-FU and leucovorin to the patient.

15. The method of embodiment 14, comprising administering 400 mg/m² leucovorin via IV administration to the patient.

16. The method of any one of embodiments 1-13, further comprising administering irinotecan, 5-FU and levoleucovorin to the patient.

17. The method of embodiment 16, comprising administering 200 mg/m² levoleucovorin via IV administration to the patient.

18. The method of any one of embodiments 1-17, comprising administering 180 mg/m² irinotecan via IV administration to the patient.

19. The method of any one of embodiments 1-18, comprising administering 400 mg/m² 5-FU via IV administration to the patient.

20. The method of any one of embodiments 1-15, and 18-19, comprising administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

21. The method of any one of embodiments 1-13, 16 and 17, comprising administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

22. The method of any one of embodiments 1-13, further comprising administering irinotecan and 5-FU to the patient.

23. The method of embodiment 22, comprising administering 180 mg/m² irinotecan via IV administration to the patient.

24. The method of embodiment 22, comprising administering 150 mg/m² irinotecan via IV administration to the patient.

25. The method of any one of embodiments 1-13 and 23, comprising administering via IV administration 180 mg/m² irinotecan, and 400 mg/m² 5-FU IV bolus and 2400 mg/m² 5-FU IV continuous infusion over 46-48 hours to the patient every two weeks.

26. The method of any one of embodiments 1-13 and 24, comprising administering via IV administration 150 mg/m² irinotecan, and 400 mg/m² 5-FU IV bolus and 2400 mg/m² 5-FU IV continuous infusion over 46-48 hours to the patient every two weeks.

27. The method of any one of embodiments 1-26, wherein the cancer is a solid tumor.

28. The method of any one of embodiments 1-27, wherein the cancer is small bowel cancer, appendiceal cancer, endometrial cancer, hepatobiliary cancer, small cell lung cancer, cervical cancer, pancreatic cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma.

29. The method of any one of embodiments 1-28, wherein the cancer is non-small cell lung carcinoma (NSCLC) or colorectal cancer (CRC).

30. The method of any one of embodiments 1-29, wherein the cancer is non-small cell lung carcinoma (NSCLC).

31. The method of any one of embodiments 1-28, wherein the cancer is metastatic pancreatic cancer.

32. The method of any one of embodiments 1-28, wherein the cancer is colorectal cancer.

33. The method of any one of embodiments 1-32, wherein the patient has previously received at least one other systemic cancer therapy.

34. The method of embodiment 33, wherein at least one systemic cancer therapy is selected from therapy with a KRAS^{G12C} inhibitor, anti-PD-1 therapy, anti-PD-L1 therapy, and platinum-based chemotherapy.

35. The method of embodiment 33, wherein the at least one systemic cancer therapy is not a therapy with a KRAS^{G12C} inhibitor.

36. The method of any one or embodiments 1-28, wherein the cancer is metastatic colorectal cancer (mCRC).

37. The method of any one of embodiments 1-36, wherein the patient has previously received at least one other systemic cancer therapy.

38. The method of any one of embodiments 1-36, wherein the patient has previously received at least two other systemic cancer therapies.

39. The method of embodiment 37 and embodiment 38, wherein the systemic cancer therapy is a therapy comprising administering to the patient fluoropyrimidine, irinotecan, and oxaliplatin.

40. The method of any one of embodiments 36-39, wherein the mCRC is determined to be MSI-H and the systemic cancer therapy is a therapy comprising administering to the patient a checkpoint inhibitor.

41. The method of any one of embodiments 36-40, wherein the mCRC comprises a BRAF V600E mutation and the systemic cancer therapy is a therapy comprising administering to the patient encorafenib and cetuximab.

42, The method of any one of embodiments 36-41, wherein the systemic cancer therapy is adjuvant therapy, and wherein the cancer has progressed on or within six months of completion of the adjuvant therapy.

43. The method of any one of embodiments 36-42, wherein the systemic cancer therapy is adjuvant therapy after resection of the mCRC.

44. The method of any one of embodiments 36-43, wherein the patient exhibits an ECOG performance status of equal or less than 2.

45. The method of any one of embodiments 36-44, wherein the patient does not have active brain metastases.

46. The method of any one of embodiments 36-45, wherein the patient does not have leptomeningeal disease.

47. The method of any one of embodiments 36-46, wherein the patient does not have a human immunodeficiency virus (HIV) infection.

48. The method of any one of embodiments 36-47, wherein the patient does not have a hepatitis B or C infection.

49. The method of any one of embodiments 37-48, wherein the systemic cancer therapy is a therapy comprising administering to the patient (i) trifluridine and tipiracil and (ii) regorafenib.

50. The method of any one of embodiments 37-49, wherein the systemic therapy is not a therapy comprising administering to the patient a KRAS^{G12C} inhibitor.

51. The method of embodiment 50, wherein the KRAS^{G12C} inhibitor is sotorasib.

52. The method of embodiment 50, wherein the KRAS^{G12C} inhibitor is adagrasib.

53. The method of any one of embodiments 37-52, wherein the systemic therapy is not a therapy comprising administering to the patient trifluridine and tipiracil.

54. The method of any one of embodiments 37-53, wherein the systemic therapy is not a therapy comprising administering to the patient regorafenib.

55. The method of any one of embodiments 1-36, wherein the patient has not previously received another systemic cancer therapy.

56. The method of embodiment 55, wherein the patient does not have active brain metastases.

57. The method of embodiment 55 and 56, wherein the patient does not have leptomeningeal disease.

58. The method of any one of embodiments 55-57, wherein the mCRC does not comprise a BRAF V600E mutation.

59. The method of any one of embodiments 55-58, wherein the mCRC is determined not to be MSI-H.

60. The method of any one of embodiments 55-59, wherein the systemic therapy is a therapy comprising administering to the patient a KRAS^{G12C} inhibitor.

61. The method of embodiment 60, wherein the KRAS^{G12C} inhibitor is sotorasib.

62. The method of embodiment 60, wherein the KRAS^{G12C} inhibitor is adagrasib.

63. The method of any one of embodiments 55-62, wherein the patient does not have a dihydropyrimidine dehydrogenase deficiency.

64. The method of any one of embodiments 55-63, wherein the patient does not have a UDP-glucuronosyltransferase 1A1 (UGT1A1)*28 homozygosity or Gilbert's disease.

65. The method of any one of embodiments 55-64, wherein the patient does not have a hepatitis B or C infection.

66. The method of any one of embodiments 55-64, wherein the patient does not have a New York Heart Association cardiac disease of class II or greater, myocardial infarction less than 6 months prior to treatment, unstable arrhythmias, or unstable angina.

67. The method of any one of embodiments 55-64, wherein the patient exhibits an ECOG performance status of equal or less than 1.

68. The method of any one of embodiments 1-36, wherein the patient has previously received one other systemic cancer therapy.

69. The method of embodiment 68, wherein the cancer is determined to be MSI-H, and the one other systemic cancer therapy is a checkpoint inhibitor.

70. The method of embodiment 68 or embodiment 69, wherein the patient has received the one other systemic cancer therapy and progressed on or after said therapy.

71. The method of any one of embodiments 68-70, wherein the one other systemic cancer therapy is adjuvant therapy, and wherein the mCRC has progressed on or within six months of completion of the adjuvant therapy.

72. The method of any one of embodiments 68-70, wherein the mCRC is determined to be MSI-H and the one other systemic cancer therapy is a therapy comprising administering a checkpoint inhibitor.

73. The method of any one of embodiments 68-70, wherein the one other systemic therapy is not a therapy comprising administering to the patient a KRAS^{G12C} inhibitor.

74. The method of embodiment 73, wherein the KRAS^{G12C} inhibitor is sotorasib.

75. The method of embodiment 73, wherein the KRAS^{G12C} inhibitor is adagrasib.

76. The method of any one of embodiments 68-70, wherein one other systemic therapy is not a therapy comprising administering irinotecan.

77. The method of any one of embodiments 68-76, wherein the patient exhibits an ECOG performance status of equal or less than 1.

78. The method of any one of embodiments 68-77, wherein the patient does not have active brain metastases.

79. The method of any one of embodiments 68-78, wherein the patient does not have leptomeningeal disease.

80. The method of any one of embodiments 68-79, wherein the mCRC does not comprise a BRAF V600E mutation.

81. The method of any one of embodiments 68-80, wherein the patient does not have a dihydropyrimidine dehydrogenase deficiency.

82. The method of any one of embodiments 68-81, wherein the patient does not have a UDP-glucuronosyltransferase 1A1 (UGT1A1)*28 homozygosity or Gilbert's disease.

83. The method of any one of embodiments 68-82, wherein the patient does not have a hepatitis B or C infection.

84. The method of any one of embodiments 68-83, wherein the patient does not have a New York Heart Association cardiac disease of class II or greater, myocardial infarction less than 6 months prior to treatment, unstable arrhythmias, or unstable angina.

85. The method of any one of embodiments 1-84, wherein the patient exhibits at least a stable disease (SD) after 1, 3, or 6 months of sotorasib and panitumumab therapy, as measured by RECIST 1.1 protocol.

86. The method of any one of embodiments 1-85, wherein the patient exhibits at least a partial response (PR) after 1, 3, or 6 months of sotorasib and panitumumab therapy, as measured by RECIST 1.1 protocol.

87. The method of any one of embodiments 1-86, wherein the patient exhibits a progression free survival (PFS) of at least 3 months.

88. The method of any one of embodiments 1-87, wherein the patient is in further need of treatment with an acid-reducing agent.

89. The method of embodiment 88, wherein the acid-reducing agent is a proton pump inhibitor (PPI), a H2 receptor antagonist (H2RA), or a locally acting antacid.

90. The method of embodiment 88 or embodiment 89, wherein the acid-reducing agent is a locally acting antacid, and wherein sotorasib is administered about 4 hours before or about 10 hours after the locally acting antacid.

91. The method of embodiment 89 or embodiment 90, wherein the locally acting antacid is sodium bicarbonate, calcium carbonate, aluminum hydroxide, or magnesium hydroxide.

92. The method of any one of embodiments 1-91, wherein the patient is in further need of treatment with a proton pump inhibitor (PPI) or H2 receptor antagonist (H2RA).

93. The method of embodiment 92, wherein the patient is not administered a PPI or a H2RA in combination with sotorasib.

94. The method of any one of embodiments 89, 92, or 93, wherein the PPI is omeprazole, pantoprazole, esomeprazole, lansoprazole, rabeprazole, or dexlansoprazole.

95. The method of any one of embodiments 89, 92, or 93, wherein the H2RA is famotidine, ranitidine, cimetidine, nizatidine, roxatidine, or lafutidine.

96. The method of any one of embodiments 1-95, wherein the patient is in further need of treatment with a CYP3A4 inducer.

97. The method of embodiment 96, wherein the patient is not administered a CYP3A4 inducer in combination with sotorasib.

98. The method of embodiment 96 or 97, wherein the CYP3A4 inducer is a barbiturate, brigatinib, carbamazepine, clobazam, dabrafenib, efavirenz, elagolix, enzalutamide, eslicarbazepine, glucocorticoid, letermovir, lorlatinib, modafinil, nevirapine, oritavancin, oxcarbazepine, perampanel, phenobarbital, phenytoin, pioglitazone, rifabutin, rifampin, telotristat, or troglitazone.

99. The method of embodiment 96 or embodiment 97, wherein the patient is not administered a strong CYP3A4 inducer in combination with sotorasib.

100. The method of embodiment 99, wherein the strong CYP3A4 inducer is phenytoin or rifampin.

101. The method of any one of embodiments 1-100, wherein the patient is in further need of treatment with a CYP3A4 substrate.

102. The method of embodiment 101, wherein the patient is not administered a CYP3A4 substrate in combination with sotorasib.

103. The method of embodiment 101 or 102, wherein the CYP3A4 substrate is abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus(fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, or ziprasidone.

104. The method of any one of embodiments 1-103, wherein the patient is in further need of treatment with a P-glycoprotein (P-gp) substrate.

105. The method of embodiment 104, wherein the patient is not administered a P-gp substrate in combination with sotorasib.

106. The method of embodiment 104 or embodiment 105, wherein the P-gp substrate is etexilate, digoxin, or fexofenadine.

107. The method of any one of embodiments 1-106, wherein the cancer exhibits a PD-L1 tumor proportion score (TPS) of 1-49%.

108. The method of any one of embodiments 1-107, wherein the cancer exhibits a PD-L1 tumor proportion score (TPS) of less than 1%.

109. The method of any one of embodiments 1-108, wherein the cancer exhibits a PD-L1 tumor proportion score (TPS) of 50-100%.

110. The method of any one of embodiments 1-109, wherein the cancer further comprises a STK11 mutation.

111. The method of any one of embodiments 1-110, wherein the cancer further comprises a KEAP1 mutation.

112. The method of embodiment 110 or embodiment 111, wherein the mutation is a loss-of-function mutation.

### Second Set of Alternative Embodiments

1. A method of treating cancer comprising a KRAS G12C mutation in a patient comprising administering to the patient (a) sotorasib and (b) an anti-epidermal growth factor receptor (EGFR) antibody in amounts effective to treat the cancer.

2. The method of embodiment 1, comprising administering 960 mg sotorasib to the patient daily.

3. The method of embodiment 1, comprising administering 240 mg sotorasib to the patient daily.

4. The method of any one of embodiments 1-3, comprising administering sotorasib to the patient once daily.

5. The method of any one of embodiments 1-3, comprising administering sotorasib to the patient twice daily.

6. The method of any one of embodiments 1-5, wherein the anti-EGFR antibody is panitumumab.

7. The method of embodiment 6, comprising administering 6 mg/kg panitumumab to the patient.

8. The method of embodiment 6 or embodiment 7, comprising administering to the patient
(a) 960 mg sotorasib daily; and
(b) 6 mg/kg panitumumab via IV administration every two weeks.

9. The method of embodiment 6 or embodiment 7, comprising administering to the patient
(a) 240 mg sotorasib daily; and
(b) 6 mg/kg panitumumab via IV administration every two weeks.

10. The method of any one of embodiments 1-13, further comprising administering (c) irinotecan, (d) 5-FU and (e) leucovorin or levoleucovorin to the patient.

11. The method of embodiment 10, comprising administering 400 mg/m² leucovorin via IV administration to the patient.

12. The method of embodiment 10, comprising administering 200 mg/m² levoleucovorin via IV administration to the patient.

13. The method of any one of embodiments 10-12, comprising administering 180 mg/m² irinotecan via IV administration to the patient.

14. The method of any one of embodiments 10-13, comprising administering 400 mg/m² 5-FU via IV administration to the patient.

15. The method of embodiment 10, comprising administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

16. The method of embodiment 10, comprising administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

17. The method of any one of embodiments 1-16, wherein the cancer is a solid tumor.

18. The method of any one of embodiments 1-17, wherein the cancer is non-small cell lung cancer (NSCLC).

19. The method of any one of embodiments 1-17, wherein the cancer is metastatic pancreatic cancer.

20. The method of any one of embodiments 1-17, wherein the cancer is colorectal cancer.

21. The method of any one or embodiments 1-17, wherein the cancer is metastatic colorectal cancer (mCRC).

22. The method of any one of embodiments 1-36, wherein the patient has received at least one prior systemic cancer therapy.

23. The method of any one of embodiments 1-36, wherein the patient has received at least two prior systemic cancer therapies.

24. The method of embodiment 22 and embodiment 23, wherein the systemic cancer therapy is a therapy comprising administering to the patient fluoropyrimidine, irinotecan, and oxaliplatin.

25. The method of any one of embodiments 21-24, wherein the mCRC is determined to be MSI-H and the systemic cancer therapy is a therapy comprising administering to the patient a checkpoint inhibitor.

26. The method of any one of embodiments 21-25, wherein the mCRC comprises a BRAF V600E mutation and the systemic cancer therapy is a therapy comprising administering to the patient encorafenib and cetuximab.

27. The method of any one of embodiments 21-26, wherein the patient exhibits an ECOG performance status of equal or less than 2.

28. The method of any one of embodiments 21-27, wherein the patient does not have active brain metastases.

29. The method of any one of embodiments 22-28, wherein the systemic therapy is not a therapy comprising administering to the patient a KRAS^{G12C} inhibitor.

30. The method of any one of embodiments 1-21, wherein the patient has not received a prior systemic cancer therapy.

31. The method of embodiment 30, wherein the patient does not have active brain metastases.

32. The method of embodiment 30 or embodiment 31, wherein the mCRC does not comprise a BRAF V600E mutation.

33. The method of any one of embodiments 30-32, wherein the mCRC is determined not to be MSI-H.

34. The method of any one of embodiments 30-33, wherein the systemic therapy is a therapy comprising administering to the patient a KRAS^{G12C} inhibitor.

35. The method of any one of embodiments 30-34, wherein the patient exhibits an ECOG performance status of equal or less than 1.

36. The method of any one of embodiments 1-21, wherein the patient has received one prior systemic cancer therapy.

37. The method of embodiment 36, wherein if the cancer is determined to be MSI-H, then the systemic cancer therapy is a checkpoint inhibitor.

38. The method of embodiment 36 or embodiment 37, wherein the patient has received the systemic cancer therapy and progressed on or after said therapy.

39. The method of any one of embodiments 36-38, wherein the systemic therapy is not a therapy comprising administering to the patient a KRAS^{G12C} inhibitor.

40. The method of any one of embodiments 36-38, wherein the systemic therapy is not a therapy comprising administering irinotecan.

41. The method of any one of embodiments 36-40, wherein the patient exhibits an ECOG performance status of equal or less than 1.

42. The method of any one of embodiments 36-41, wherein the patient does not have active brain metastases.

43. The method of any one of embodiments 36-42, wherein the mCRC does not comprise a BRAF V600E mutation.

44. The method of any one of embodiments 1-43, wherein the patient exhibits at least a stable disease (SD) after 1, 3, or 6 months of sotorasib and panitumumab therapy, as measured by RECIST 1.1 protocol.

45. The method of any one of embodiments 1-43, wherein the patient exhibits at least a partial response (PR) after 1, 3, or 6 months of sotorasib and panitumumab therapy, as measured by RECIST 1.1 protocol.

46. The method of any one of embodiments 1-45, wherein the patient is in further need of treatment with an acid-reducing agent.

47. The method of embodiment 46, wherein the acid-reducing agent is a proton pump inhibitor (PPI), a H2 receptor antagonist (H2RA), or a locally acting antacid.

48. The method of embodiment 46 or embodiment 47, wherein the acid-reducing agent is a locally acting antacid, and wherein sotorasib is administered about 4 hours before or about 10 hours after the locally acting antacid.

49. The method of any one of embodiments 1-48, wherein the patient is in further need of treatment with a proton pump inhibitor (PPI) or H2 receptor antagonist (H2RA).

50. The method of embodiment 49, wherein the patient is not administered a PPI or a H2RA in combination with sotorasib.

### Examples

The term “subject” is used throughout the Examples interchangeably with “patient”, who is in need of treatment with one or more methods described herein.

### Example 1—Sotorasib in Combination with Panitumumab and Optionally FOLFIRI

Without wishing to be bound by any particular theory, the following is noted: Sotorasib at 960 mg QD was shown to be safe and effective under study conditions under Study 20170543 (https://clinicaltrials.gov/ct2/show/NCT03600883; CodeBreaK100). Since resistance to sotorasib may be mediated by upregulation of signaling through epidermal growth factor receptor (EGFR) pathway, adding an EGFR inhibitor to sotorasib therapy may block bypass activation of the mitogen activated kinase (MAPK) signaling and lead to improved anti-tumor activity. FOLFIRI is chosen as the chemotherapy backbone as it has been combined successfully with panitumumab in a phase 3 study of metastatic colorectal cancer (Peeters et al, 2010). This study, Study 20190135 (https://clinicaltrials.gov/ct2/show/NCT04185883; CodeBreaK 101, Subprotocol H, will therefore explore sotorasib in combination with panitumumab (EGFR targeted monoclonal antibody) and optionally FOLFIRI.

### Overall Design:

A multicenter, open label study is set up to evaluate the safety, tolerability, pharmacokinetics (PK), PD, and efficacy of sotorasib in combination with panitumumab (or panitumumab plus FOLFIRI) in subjects with KRAS G12C mutant advanced CRC, NSCLC, and advanced solid tumors.

On days when PK samples are drawn in cycle 1, and after any dose hold of sotorasib, the treatments will be administered in the following sequence: sotorasib, panitumumab, and FOLFIRI if applicable. Sotorasib will be administered orally once daily (QD). Alternatively, twice daily dosing may be used but the total daily dose will be the same. Panitumumab 6 mg/kg will be administered as 60-minute (≤1000 mg) or 90-minute (>1000 mg) intravenous (IV) infusion every 2 weeks (Q2W). FOLFIRI consists of 180 mg/m² irinotecan and 400 mg/m² racemic leucovorin by IV infusion on day 1 and 5-fluorouracil (5-FU) 400 mg/m² IV bolus on day 1, followed by 2400 mg/m² continuous infusion administered over 46 to 48 hours beginning on day 1 given Q2W. Levoleucovorin at 200 mg/m² may be used instead of racemic leucovorin.

The first dose of panitumumab will be administered 2 hours after sotorasib administration. Subsequent panitumumab doses may be administered immediately following sotorasib administration. In Part 1 Cohort B, FOLFIRI will be administered after panitumumab.

The study will include a dose exploration phase (Part 1) and expansion phase (Part 2). Part 1 Cohort A is a dose exploration period to examine the safety of combining sotorasib with panitumumab. Sotorasib dose will start at 960 mg total daily dose. Two lower dose levels of sotorasib and 1 lower dose level of panitumumab can be explored if needed. Part 1 Cohort B will consist of dose exploration of sotorasib, panitumumab and FOLFIRI and will commence once the recommended phase 2 dose (RP2D) of sotorasib and panitumumab is defined in Part 1 Cohort A. Part 2, consisting of 8 separate cohorts, will commence once the dose of the sotorasib and panitumumab combination is defined in Part 1 Cohort A, however certain cohorts will commence once the dose of sotorasib, in combination with panitumumab plus FOLFIRI, is defined in Part 1 Cohort B. The 8 cohorts in Part 2 (i.e., cohorts A-H) are as follows:

### Dose Expansion Cohorts for Sotorasib+Panitumumab

Cohort A: KRAS^{G12C} inhibitor naive KRAS G12C mutated metastatic colorectal cancer previously treated with fluoropyrimidine, oxaliplatin, irinotecan, and anti-angiogenic agent and if microsatellite instability high (MSI H), a checkpoint inhibitor (CPI) if approved in the region (n=up to 40).

Cohort B: Any KRAS G12C mutated solid tumor refractory to KRAS^{G12C} inhibitor therapy (n=up to 20).

Cohort C: KRAS^{G12C} inhibitor naïve KRAS G12C mutated NSCLC previously treated with at least 1 prior systemic therapy (n=up to 40).

Cohort D: KRAS^{G12C} inhibitor naïve KRAS G12C mutated metastatic colorectal cancer previously treated with 1 prior line of therapy for metastatic disease (n=up to 20). The opening of this cohort will be contingent on the activity of this combination in other cohorts of this study and on emerging sotorasib combination data.

Cohort E: (twice daily [BID] cohort): KRAS^{G12C} inhibitor naïve KRAS G12C mutated metastatic colorectal cancer previously treated with fluoropyrimidine, oxaliplatin, irinotecan, and anti-angiogenic agent and if MSI-H, a CPI if approved in the region (n=up to 40). The opening of this cohort will be contingent on the activity of sotorasib and panitumumab in other cohorts of this study and on emerging sotorasib monotherapy and combination data.

Cohort H: KRAS^{G12C} inhibitor naïve KRAS G12C mutated pancreatic cancer with at least one prior therapy for metastatic disease or refuse or is ineligible for standard of care chemotherapy (n=up to 40).

Dose expansion cohort for sotorasib+panitumumab+FOLFIRI

Cohort F: KRAS^{G12C} inhibitor naïve KRAS G12C mutated metastatic colorectal cancer with no prior therapy for metastatic disease (n=up to 40)

Cohort G: KRAS^{G12C} inhibitor naïve KRAS G12C mutated metastatic colorectal cancer with at least one prior therapy for metastatic disease.

At least 2 responses must be seen in the first 20 subjects in Cohorts A, C, E, F, G or H in Part 2 to continue enrollment in the respective cohort. Part 2 will confirm safety and evaluate preliminary anti-tumor activities of sotorasib in combination with panitumumab, and sotorasib in combination with panitumumab and FOLFIRI.

Overall, approximately 310 subjects will be enrolled in the study across both Part 1 and Part 2.

### Part 1: Dose Exploration

The primary objective of Part 1 is to evaluate the safety and tolerability of sotorasib in combination with panitumumab and sotorasib in combination with panitumumab plus FOLFIRI. Accordingly, the primary endpoint will include an assessment of dose limiting toxicities, treatment-emergent and treatment-related adverse events.

### Part 1, Cohort A

Dose Level 1: 960 mg sotorasib orally total daily dose+6 mg/kg panitumumab IV on day 1 Q2W
Dose Level −1: 720 mg sotorasib orally total daily dose+6 mg/kg panitumumab IV on day 1 Q2W
Dose Level −2: 480 mg sotorasib orally total daily dose+6 mg/kg panitumumab IV on day 1 Q2W

If dose level 1 is not tolerable and deemed primarily due to panitumumab toxicity (such as rash, paronychia, etc.), the dose of panitumumab 3 mg/kg IV day 1 Q2W can be explored with or without a dose reduction of sotorasib.

### Part 1, Cohort B

Dose Level 1: Sotorasib orally total daily dose identified from Part 1 Cohort A+6 mg/kg panitumumab IV (or 3 mg/kg if that is the dose identified in Part 1 Cohort A) on day 1 Q2W±irinotecan 180 mg/m² on day 1, leucovorin 400 mg/m² on day 1 and 5-FU 400 mg/m² IV bolus on day 1 and 2400 mg/m² IV continuous infusion (IVCI) over 46 to 48 hours beginning on day 1 given Q2W. For investigative sites that use levoleucovorin instead of racemic leucovorin, the levoleucovorin dose will be 200 mg/m².
Dose Level −1: Sotorasib orally total daily dose identified from Part 1 Cohort A+6 mg/kg panitumumab IV (or 3 mg/kg if that is the dose identified in Part 1 Cohort A) on day 1 Q2W±irinotecan 180 mg/m² on day 1, and 5 FU 2400 mg/m² IV continuous infusion (IVCI) over 46 to 48 hours beginning on day 1 given Q2W.
Dose Level −2: Sotorasib orally total daily dose identified from Part 1 Cohort A+6 mg/kg panitumumab IV (or 3 mg/kg if that is the dose identified in Part 1 Cohort A) on day 1 Q2W±irinotecan 150 mg/m² on day 1, and 5 fluorouracil 2400 mg/m² IV continuous infusion (IVCI) over 46 to 48 hours beginning on day 1 given Q2W.

### Part 2—Dose Expansion for Sotorasib+Panitumumab (and +FOLFIRI, Cohorts F and G Only)

The primary objective of Part 2 is to evaluate the safety and preliminary anti-tumor activity of sotorasib in combination with panitumumab in KRAS^{G12C} inhibitor naïve KRAS G12C mutated metastatic CRC with previous chemotherapy and anti-angiogenic agent treatment and if MSI-H, a CPI if approved in the region (Cohorts A and E); in any KRAS G12C mutated advanced solid tumors with previous exposure and progression on a KRAS G12C inhibitor (Cohort B); in KRAS^{G12C} inhibitor naïve KRAS G12C mutated NSCLC with at least 1 prior systemic therapy (Cohort C); and in KRAS^{G12C} inhibitor naïve KRAS G12C mutated metastatic CRC with no more than 1 prior line of therapy for metastatic disease (Cohort D); and in KRAS^{G12C} inhibitor naïve KRAS G12C mutated metastatic pancreatic cancer with at least 1 prior treatment for advance disease or refused or are ineligible for standard of care chemotherapy (Cohort H). Sotorasib in combination with panitumumab and FOLFIRI will also be evaluated in KRAS^{G12C} inhibitor naïve KRAS G12C mutated metastatic CRC with no prior therapy for metastatic disease (Cohort F) and with at least one prior therapy for metastatic disease (Cohort G).

Objective response rate (ORR) measured by RECIST 1.1 will provide the initial evidence of anti-tumor activity and will be included as the secondary endpoint for this part of the study. Other related measures of efficacy including PFS, duration of response, disease control rate, and time to response will provide additional supportive evidence of anti-tumor activity and will be included as secondary endpoints.

Cohort A: KRAS^{G12C} inhibitor naïve KRAS G12C mutated metastatic colorectal cancer previously treated with fluoropyrimidine, oxaliplatin, irinotecan, and anti angiogenic agent and if MSI-H, a CPI if approved in the region: Sotorasib total daily dose identified from Part 1 Cohort A of this study orally+6 mg/kg (or 3 mg/kg if that is dose identified in part 1 Cohort A) panitumumab IV on day 1 Q2W.

Cohort B: Any KRAS G12C mutated solid tumor refractory to KRAS^{G12C} inhibitor therapy: Sotorasib total daily dose identified from Part 1 Cohort A of this study orally+6 mg/kg (or 3 mg/kg if that is dose identified in part 1 Cohort A) panitumumab IV on day 1 Q2W.

Cohort C: KRAS^{G12C} inhibitor naïve KRAS G12C mutated NSCLC treated with at least 1 prior systemic therapy: Sotorasib total daily dose identified from Part 1 Cohort A of this study orally+6 mg/kg (or 3 mg/kg if that is the dose identified in Part 1 Cohort A) panitumumab IV on day 1 Q2W.

Cohort D: KRAS^{G12C} inhibitor naïve KRAS G12C mutated metastatic CRC with no more than 1 prior line of therapy for metastatic disease: Sotorasib total daily dose identified from Part 1 Cohort A of this study orally+6 mg/kg (or 3 mg/kg if that is the dose identified in Part 1 Cohort A) panitumumab IV on day 1 Q2W. The opening of this cohort will be contingent on the activity of this combination in other cohorts of this study and on emerging sotorasib combination data.

Cohort E (BID cohort): KRAS^{G12C} inhibitor naïve KRAS G12C mutated metastatic colorectal cancer previously treated with fluoropyrimidine, oxaliplatin, irinotecan, and anti angiogenic agent and if MSI-H, a CPI if approved in the region, in subjects receiving sotorasib on a BID dosing schedule: Sotorasib total daily dose identified from Part 1 Cohort A of this study orally+6 mg/kg (or 3 mg/kg if that is the dose identified in Part 1 Cohort A) panitumumab IV on day 1 Q2W. The opening of this cohort will be contingent on the activity of sotorasib and panitumumab in other cohorts of this study and on emerging sotorasib monotherapy and combination data.

Cohort H: KRAS^{G12C} inhibitor naïve KRAS G12C mutated pancreatic cancer with at least one prior therapy for metastatic disease or refused or is ineligible for standard of care chemotherapy: Sotorasib total daily dose identified from Part 1 Cohort A of this study orally with 6 mg/kg (or 3 mg/kg if that is the dose identified in Part 1 Cohort A) panitumumab IV on day 1 Q2W.

Cohort F: KRAS^{G12C} inhibitor naïve KRAS G12C mutated metastatic colorectal cancer with no prior therapy for metastatic disease: Sotorasib total daily dose identified from Part 1 Cohort B of this study orally+6 mg/kg (or 3 mg/kg if that is the dose identified in Part 1 Cohort B) panitumumab IV on day 1 Q2W+FOLFIRI dose identified in Part 1 Cohort B.

Cohort G: KRAS^{G12C} inhibitor naïve KRAS G12C mutated metastatic CRC with at least one prior therapy for metastatic disease. Sotorasib total daily dose identified from Part 1 Cohort B of this study orally+6 mg/kg (or 3 mg/kg if that is the dose identified in Part 1 Cohort B) panitumumab IV on day 1 Q2W+FOLFIRI dose identified in Part 1 Cohort B.

The duration of this study for subjects will be approximately 3 years. The duration of screening is up to 28 days. The planned length of treatment for a subject will be until disease progression or unacceptable toxicity. The duration of treatment for an individual subject is anticipated to be approximately 8 months followed by a safety follow up (SFU) visit that occurs 30 (+3) days after the last dose of investigational product or protocol mandated therapies. Subjects will be followed until the analysis of PFS or 3 years after the last subject has enrolled, whichever is later.

### Summary of Subject Eligibility Criteria:

Adult subjects (>18 years old) with metastatic advanced solid tumors with KRAS G12C mutation as assessed by molecular testing of tumor biopsy specimens and have received at least 1 prior systemic therapy for advanced disease will be eligible to participate in the study for Part 1 Cohorts A and B and Part 2 Cohorts A to E, and H. Subjects in Part 2 Cohort F cannot have received prior therapy for metastatic disease. Subjects in Part 2 Cohort G must have received at least one prior therapy for metastatic disease. Study cohort eligibility criteria are as follows.

### Part 1 Cohort A:

Pathologically documented, metastatic colorectal cancer with KRAS G12C mutation identified through molecular testing performed according to in-country requirements. In the United States, this test must be performed in a Clinical Laboratory Improvement Amendments (CLIA)-certified laboratory.
Subjects must not have required dose reduction or been intolerant of a KRAS^{G12C} inhibitor if they have received treatment with a KRAS^{G12C} inhibitor in the past. A minimum of 2 subjects must be KRAS^{G12C}inhibitor naïve per dose level.
Subjects must have had at least 1 prior treatment for advanced disease

### Part 1 Cohort B:

Pathologically documented, metastatic colorectal cancer with KRAS G12C mutation identified through molecular testing performed according to in-country requirements. In the United States, this test must be performed in a CLIA-certified laboratory
Subjects must not have required dose reduction or been intolerant of a KRAS^{G12C} inhibitor if they have received treatment with a KRAS^{G12C} inhibitor in the past. A minimum of 2 subjects must be KRAS^{G12C}inhibitor naïve per dose level.
If subject had received prior treatment with FOLFIRI, subjects must not have required a dose reduction for any component of the FOLFIRI regimen because of toxicity.
Subjects must have had at least 1 prior treatment for advanced disease.

### Part 2 Cohort A:

Pathologically documented, metastatic colorectal cancer with KRAS G12C mutation identified through molecular testing performed according to in-country requirements. In the United States, this test must be performed in a CLIA-certified laboratory
Subjects may not have received treatment with a KRAS^{G12C} inhibitor in the past.
Subjects must have progressed after receiving fluoropyrimidine, oxaliplatin, and irinotecan and an anti-angiogenic agent.
For those subjects with tumors known to be MSI-H, prior therapy with a CPI is required if they were clinically able to receive a CPI, and if one of these agents is approved for that indication in the region or country.

### Part 2 Cohort B:

Pathologically documented metastatic advanced solid tumor with KRAS G12C mutation identified through molecular testing performed according to in-country requirements. In the United States, this test must be performed in a CLIA-certified laboratory.
Subjects must not have been intolerant of a KRAS^{G12C} inhibitor in the past.
If subject previously required sotorasib dose reduction, the subject may be eligible with medical monitor approval, if the investigator assesses that treatment may be beneficial, and if the previous dose reduction was for toxicity that may not be due to sotorasib in the investigator's opinion.
Subjects must have progressed on or within 2 months of last dose of KRAS^{G12C} inhibitor.
Subjects must have had at least 1 prior treatment for advanced disease, and at least 1 of the prior treatments must have included a KRAS^{G12C} inhibitor.

### Part 2 Cohort C:

Pathologically documented, metastatic NSCLC with KRAS G12C mutation identified through molecular testing performed according to in country requirements. In the United States, this test must be performed in a CLIA-certified laboratory
Subjects may not have received treatment with a KRAS^{G12C} inhibitor in the past.
Subjects must have had at least 1 prior treatment for advanced disease.

### Part 2 Cohort D:

Pathologically documented, metastatic CRC with KRAS G12C mutation identified through molecular testing performed according to in-country requirements. In the United States, this test must be performed in a CLIA-certified laboratory.
Subjects may not have received treatment with a KRAS^{G12C} inhibitor in the past.
Subjects must have received 1 and no more than 1 prior regimen for metastatic disease.

### Part 2 Cohort E (BID Dosing):

Pathologically documented, metastatic colorectal cancer with KRAS G12C mutation identified through molecular testing performed according to in-country requirements.
Subjects may not have received treatment with a KRAS^{G12C} inhibitor in the past.
Subjects must have progressed after receiving fluoropyrimidine, oxaliplatin, and irinotecan and an anti-angiogenic agent.
For those subjects with tumors known to be MSI-H, prior therapy with an anti-PD1 therapy is required if they were clinically able to receive an anti PD1 therapy and if one of these agents is approved for that indication in the region or country.

### Part 2 Cohort H:

Pathologically documented, metastatic pancreatic cancer with KRAS G12C mutation identified through molecular testing performed according to in-country requirements. In the United States, this test must be performed in a CLIA-certified laboratory
Subjects may not have received treatment with a KRAS^{G12C} inhibitor in the past
Subjects must have had at least 1 prior treatment for metastatic disease or have refused standard of care chemotherapy or standard of care chemotherapy is contraindicated.
Neoadjuvant or adjuvant therapy will count as a line of therapy for metastatic disease if the subject progressed on or within 6 months of completion of neoadjuvant or adjuvant therapy administration.

### Part 2 Cohort F:

Pathologically documented, metastatic CRC with KRAS G12C mutation identified through molecular testing performed according to in-country requirements.
Subjects may not have received treatment with a KRAS^{G12C} inhibitor in the past.
Subjects may not have received any prior systemic therapy for metastatic disease.

### Part 2 Cohort G:

Pathologically documented, metastatic CRC with KRAS G12C mutation identified through molecular testing performed according to in-country requirements.
Subjects may not have received treatment with a KRAS^{G12C} inhibitor in the past.
Subjects must have received at least one prior systemic therapy for metastatic disease.

Subjects must be willing to undergo pretreatment tumor biopsy and tumor biopsy on treatment, if clinically feasible. If a tumor biopsy prior to treatment is not medically feasible, or if the sample has insufficient tissue for testing, subjects must be willing to provide archived tumor tissue samples (formalin-fixed, paraffin-embedded [FFPE] sample) collected within the past 5 years, if available. Subjects with prior molecularly confirmed KRAS G12C mutation who do not have archived tissue available can be allowed to enroll without undergoing tumor biopsy upon agreement with investigator and the Medical Monitor if a tumor biopsy is not clinically feasible.

Measurable disease per Response Evaluation Criteria in Solid Tumors Version 1.1 (RECIST v1.1) criteria.

Eastern Cooperative Oncology Group (ECOG) Performance Status of ≤2.

Life expectancy of >3 months, in the opinion of the investigator.

Ability to take oral medications and willing to record daily adherence to investigational product.

Corrected QT interval (QTc) ≤470 msec for females and ≤450 msec for males (based on average of screening electrocardiogram triplicates).

Subjects have adequate hematological, renal and hepatic function and coagulation. Adequate hematological laboratory assessments, are as follows:

$$\begin{matrix}Absolute neutrophil count ( ANC ) \geq 1.5 \times {10}^{9} / L \\ Platelet count \geq 100 \times {10}^{9} / L \\ Hemoglobin \geq 9 \times g / dL\end{matrix}$$

Adequate renal laboratory assessments include measured creatinine clearance or estimated glomerular filtration rate based on Modification of Diet in Renal Disease (MDRD) calculation ≥60 mL/min/1.73 m².

Adequate hepatic laboratory assessments are as follows:
AST <2.5×upper limit of normal (ULN) (if liver metastases are present, ≤5×ULN)
ALT <2.5×ULN (if liver metastases are present, ≤5×ULN)
Total bilirubin <1.5×ULN (<2.0×ULN for Part 1: Cohort A, Part 2: Cohorts A to E and Cohort H
Total bilirubin ≤1×ULN for Part 1:Cohort B, and Part 2: Cohort F and Cohort G

### Adequate Coagulation Laboratory Assessments are as Follows:

Prothrombin time (PT) or activated partial thromboplastin time (PTT) or activated PTT <1.5×ULN, OR International normalized ratio (INR)<1.5×ULN or within target range if on prophylactic anticoagulation therapy.

### Exclusion Criteria:

Disease Related

Primary brain tumor.

Active brain metastases and/or carcinomatous meningitis from non-brain tumors. The phrase “active brain metastases” as used herein refers to a cancer that has spread from the original (primary, non-brain) tumor to the brain. Active brain metastases can be assessed by the presence of intracranial lesions. It is to be understood that while “metastases” is plural, patients exhibiting only one intracranial lesion under the criteria noted below is a patient who has “active brain metastases.” In some embodiments, a patient having active brain metastases has at least one measurable intracranial lesion >5 mm. In some embodiments, a patient having active brain metastases has at least one measurable intracranial lesion >5 mm but <10 mm. In some embodiments, a patient having active brain metastases has at least one measurable intracranial lesion >10 mm. A patient is not considered a patient with active brain metastases if the patient has had brain metastases or have received radiation therapy ending at least 4 weeks prior to study day 1 and are eligible if they meet all of the following criteria: a) residual neurological symptoms grade ≤2; b) on stable doses of dexamethasone, if applicable; and c) follow-up MRI performed within 30 days shows no new lesions appearing. For determining the grade of any neurological symptom attributable to an intracranial lesion, see National Cancer Institute Common Terminology Criteria for Adverse Events v5.0 (NCI CTCAE) published Nov. 27, 2017 by the National CancerInstitute, incorporated herein by reference in its entirety.

### Other Medical Conditions

History or presence of hematological malignancies unless curatively treated with no evidence of disease for ≥2 years.

History of other malignancy within the past 2 years, with the following exceptions:
Malignancy treated with curative intent and with no known active disease present for ≥2 years before enrollment and felt to be at low risk for recurrence by the treating physician;
Adequately treated non-melanoma skin cancer or lentigo maligna without evidence of disease;
Adequately treated cervical carcinoma in situ without evidence of disease;
Adequately treated breast ductal carcinoma in situ without evidence of disease;
Prostatic intraepithelial neoplasia without evidence of prostate cancer;
Adequately treated urothelial papillary non-invasive carcinoma or carcinoma in situ.

Myocardial infarction within 6 months of study day 1, symptomatic congestive heart failure (New York Heart Association >class II), unstable angina, or cardiac arrhythmia requiring medication

Gastrointestinal (GI) tract disease causing the inability to take oral medication, malabsorption syndrome, requirement for IV alimentation, uncontrolled inflammatory GI disease (e.g., Crohn's disease, ulcerative colitis).

Exclusion of Hepatitis Infection Based on the Following Results and/or Criteria:
Positive Hepatitis B Surface Antigen (HepBsAg) (indicative of chronic Hepatitis B or recent acute hepatitis B);
Negative HepBsAg with a positive for hepatitis B core antibody;
Positive Hepatitis C virus antibody: Hepatitis C virus RNA by polymerase chain reaction (PCR) is necessary. Detectable Hepatitis C virus RNA suggests chronic hepatitis C.

Known positive test for human immunodeficiency virus (HIV).

History of interstitial pneumonitis or pulmonary fibrosis, or evidence of interstitial pneumonitis or pulmonary fibrosis.

Is unable to interrupt aspirin or other nonsteroidal anti-inflammatory drugs (NSAIDs), other than an aspirin ≤dose 1.3 g per day, for a 5-day period (8-day period for long-acting agents, such as piroxicam).

Has an active infection requiring systemic therapy.

### Prior/Concomitant Therapy

Anti-tumor therapy (chemotherapy, antibody therapy, molecular targeted therapy, retinoid therapy, hormonal therapy [except for subjects with history of breast cancer receiving adjuvant hormonal therapy], or investigational agent except for sotorasib) within 28 days of study Day 1; targeted small molecule inhibitors, within 14 days of study Day 1, unless at least 5 half-lives have passed. For Part 2 Cohort B, there is no requirement for minimum time from last sotorasib dose provided all sotorasib related toxicities have resolved to grade 1 or less.

Therapeutic or palliative radiation therapy within 2 weeks of study day 1. Subjects must have recovered from all radiotherapy related toxicity to Common Terminology Criteria for Adverse Events (CTCAE) version 5.0 grade 1 or less.

Received radiation therapy to the lung that is >30 Gy within 6 months of the first dose of trial treatment

Received cumulative radiation to >25% of bone marrow for Part 1 Cohort B, and Part 2 Cohort F, and Cohort G

Known dihydropyrimidine dehydrogenase deficiency for Part 1 Cohort B, and Part 2 Cohort F, and Cohort G

Known UDP-glucuronosyltransferase 1A1 (UGT1A1)*28 homozygosity or Gilbert's disease for Part 1 Cohort B, and Part 2 Cohort F, and Cohort G

Use of known cytochrome P450 (CYP) 3A4 sensitive substrates or P-glycoprotein (P-gp) substrates (e.g., with a narrow therapeutic window), within 14 days or 5 half-lives of the CYP3A4 or P-gp substrate or its major active metabolite, whichever is longer, prior to start of therapy. CYP3A4 sensitive substrates include abemaciclib, buspirone, isavuconazole, ridaforolimus, ABT-384, capravirine, itacitinib, saquinavir, acalabrutinib, casopitant, ivabradine, sildenafil, alfentanil, cobimetinib, ivacaftor, simeprevir, alisporivir, conivaptan, L-771,688, simvastatin, almorexant, danoprevir, levomethadyl (LAAM), sirolimus, alpha dihydroergocryptine, darifenacin, lomitapide, tacrolimus, aplaviroc, darunavir, lopinavir, terfenadine, aprepitant, dasatinib, lovastatin, ticagrelor, asunaprevir, dronedarone, lumefantrine, tilidine, atazanavir, ebastine, lurasidone, tipranavir, atorvastatin, eletriptan, maraviroc, tolvaptan, avanafil, eliglustat (in subjects CYP2D6 poor metabolizers (PMs)), midazolam, triazolam, AZD1305, elvitegravir, midostaurin, ulipristal, BIRL 355, entrectinib, naloxegol, vardenafil, blonanserin, eplerenone, neratinib, venetoclax, bosutinib, everolimus, nisoldipine, vicriviroc, brecanavir, felodipine, paritaprevir, vilaprisan, brotizolam, ibrutinib, perospirone, voclosporin, budesonide, indinavir, and quetiapine. P-gp substrates with a narrow therapeutic window include digoxin, everolimus, cyclosporine, tacrolimus, sirolimus, and vincristine. P450 (CYP) 3A4 sensitive substrates with a narrow therapeutic window include alfentanil, cyclosporine, dihydroergotamine, ergotamine, everolimus, fentanyl, pimozide, quinidine, tacrolimus, and sirolimus.

Use of strong inducers of CYP3A4 (including herbal supplements such as St. John's wort) within 14 days or 5 half-lives (whichever is longer) prior to start of therapy. Strong inducers of CYP3A4 include ombitasvir and paritaprevir and ritonavir and dasabuvir, indinavir and ritonavir, tipranavir and ritonavir, ritonavir, cobicistat, ketoconazole, troleandomycin, telaprevir, danoprevir and ritonavir, elvitegravir and ritonavir, saquinavir and ritonavir, lopinavir and ritonavir, itraconazole, indinavir, voriconazole, mifepristone, mibefradil, LCL161, clarithromycin, josamycin, lonafarnib, posaconazole, telithromycin, grapefruit juice DS3, conivaptan, tucatinib, nefazodone, ceritinib, nelfinavir, saquinavir, ribociclib, idelalisib, and boceprevir.

Use of known CYP3A4 or UGT1A1 inhibitors at least 1 week prior to starting irinotecan therapy (for Part 1 Cohort B and Part 2 Cohort F and Cohort G only). UGTA1 inhibitors include ketoconazole, atazanavir, gemfibrozil, and indinavir.

Unresolved toxicities from prior anti-tumor therapy, defined as not having resolved to Common terminology Criteria for Adverse Events (CTCAE) version 5.0 grade 0 or 1, or to levels dictated in the eligibility criteria with the exception of alopecia (or any grade allowed). Grade 2 or 3 toxicities from prior anti-tumor therapy that are considered irreversible [defined as having been present and stable for >6 months], such as oxaliplatin induced neuropathy, may be allowed if they are not otherwise described in the exclusion criteria AND there is agreement to allow by both the investigator and sponsor.

Subject unable to receive both iodinated contrast for CT scans and gadolinium contrast for MRI scans.

### Prior/Concurrent Clinical Study Experience

Currently receiving treatment in another investigational device or drug study, or less than 28 days since last intervention on another investigational device or drug study(ies), with the exception of sotorasib studies, in which case, there is no requirement for minimum time from last sotorasib dose provided all sotorasib related toxicities have resolved to grade 1 or less. Other investigational procedures while participating in this study are excluded.

### Other Exclusions

Subject has known sensitivity to any of the products or components to be administered during dosing.

Subject required dose reduction or dose delay of panitumumab in the past for toxicity.

For subjects in Part 1 Cohort B and, Part 2 Cohort F and Cohort G, subject has required dose reduction or dose delay of either 5-fluorouracil or irinotecan in any prior chemotherapy regimen in the past for toxicity to the investigator's knowledge.

Subject likely to not be available to complete all protocol-required study visits or procedures, and/or to comply with all required study procedures (e.g., Clinical Outcome Assessments) to the best of the subject and investigator's knowledge.

History or evidence of any other clinically significant disorder, condition or disease (with the exception of those outlined above) that would pose a risk to subject safety or interfere with the study evaluation, procedures or completion.

Female subjects of childbearing potential with a positive pregnancy test assessed at Screening by a serum pregnancy test and/or urine pregnancy test.

Female subject is pregnant or breastfeeding or planning to become pregnant or breastfeed during treatment and for an additional 7 days after the last dose of sotorasib.

Female subject is pregnant or lactating/breastfeeding or planning to become pregnant or breastfeed during treatment and an additional 2 months after the last dose of panitumumab.

Female subject is pregnant or lactating/breastfeeding or planning to become pregnant or breastfeed during treatment and an additional 6 months after the last dose of FOLFIRI.

Female subjects of childbearing potential unwilling to use 1 highly effective method of contraception during treatment and for an additional 7 days after the last dose of sotorasib.

Female subjects of childbearing potential unwilling to use 1 highly effective method of contraception during treatment and for an additional 2 months after the last dose of panitumumab.

Female subjects of childbearing potential unwilling to use 1 highly effective method of contraception during treatment and for an additional 6 months after the last dose of FOLFIRI.

Male subjects with a female partner of childbearing potential who are unwilling to practice sexual abstinence (refrain from heterosexual intercourse) or use contraception during treatment and for an additional 7 days after the last dose of sotorasib.

Male subjects with a pregnant partner who are unwilling to practice abstinence or use a condom during treatment and for an additional 7 days after the last dose of sotorasib.

Male subjects unwilling to abstain from donating sperm during treatment and for an additional 7 days after the last dose of sotorasib.

Male subjects with a female partner of childbearing potential who are unwilling to practice sexual abstinence (refrain from heterosexual intercourse) or use contraception during treatment and for an additional 6 months after the last dose of FOLFIRI.

Male subjects with a pregnant partner or partner who are unwilling to practice abstinence or use a condom during treatment and for an additional 6 months after the last dose of FOLFIRI.

Male subjects unwilling to abstain from donating sperm during treatment and for an additional 6 months after the last dose of FOLFIRI.

### Objectives and Endpoints

| | |
|---|---|
| Objectives | Endpoints |

Primary

| | |
|---|---|
| To evaluate the safety and tolerability of | Dose-limiting toxicities, |
| sotorasib in combination with panitumumab | treatment-emergent adverse events, |
| in adult subjects with KRAS G12C mutant | treatment-related adverse events, and |
| advanced solid tumors | changes in vital signs, |
| To evaluate the safety and tolerability of | electrocardiograms (ECGs), and |
| sotorasib in combination with panitumumab | clinical laboratory tests |
| and FOLFIRI in adult subjects with KRAS | |
| G12C mutant advanced colorectal cancer | |

Secondary

| | |
|---|---|
| To evaluate anti-tumor activity of sotorasib | Objective response rate (ORR), |
| in combination with panitumumab in adult | disease control rate (DCR), duration of |
| subjects with KRAS G12C mutant | response (DOR), time to response |
| advanced colorectal cancer | (TTR), overall survival (OS), |
| To evaluate anti-tumor activity of sotorasib | progression-free survival (PFS), |
| in combination with panitumumab in adult | measured by computed tomography |
| subjects with KRAS G12C mutant | (CT) or magnetic resonance imaging |
| advanced solid tumors refractory to KRAS | (MRI) and assessed per Response |
| G12C inhibitor therapy | Evaluation Criteria in Solid Tumors |
| To evaluate anti-tumor activity of sotorasib | Version 1.1 (RECIST 1.1). |
| in combination with panitumumab in adult | |
| subjects with KRAS G12C mutant | |
| advanced non-small-cell lung cancer (NSCLC) | |
| To evaluate anti-tumor activity of sotorasib | |
| in combination with panitumumab plus | |
| FOLFIRI in adult subjects with KRAS G12C | |
| mutant advanced colorectal cancer | |
| To characterize pharmacokinetics (PK) of | Pharmacokinetic parameters of |
| sotorasib in combination with panitumumab | sotorasib including, but not limited to, |
| and in combination with panitumumab and | maximum plasma concentration (Cₘₐₓ), |
| FOLFIRI in adult subjects with KRAS G12C | time to maximum plasma |
| advanced solid tumors | concentration (tₘₐₓ), and area under |
| | the plasma concentration-time curve |
| | (AUC) |

Exploratory

| | |
|---|---|
| To investigate potential biomarkers by | Quantification of biomarker expression at |
| biochemical and/or genetic analysis of blood | protein, RNA, and DNA levels, as appropriate |
| and/or tumor tissue samples. | in blood and/or tumor samples |
| To investigate potential interaction of | Quantify plasma levels of irinotecan and SN- |
| irinotecan and sotorasib | 38 |

### FOLFIRI Regimen Pre-Medication and Supportive Medications

Prior to the administration of FOLFIRI, all subjects should receive antiemetic agents (e.g., oral or IV dexamethasone, 5-hydroxyyptamine3 [5-HT3] receptor antagonists). Antiemetic agents should be given on the day of treatment, starting at least 30 minutes prior to administration of irinotecan. Alternative and additional antiemetics may be used, where clinically indicated, at the discretion of the investigator or according to standard institutional or regional practice. Prophylactic or therapeutic administration of IV or subcutaneous atropine for cholinergic symptoms may be used at the discretion of the investigator or according to standard institutional or regional practice.

Growth factor support may be used at the discretion of the investigator or according to standard institutional or regional practice.

Medications for diarrhea management should be used at the discretion of the investigator or according to standard institutional or regional practice.

Excluded Treatments, Medical Devices, and/or Procedures During Study Period

Anti-tumor therapy such as chemotherapy, antibody therapy, molecular targeted therapy, retinoid therapy, or hormonal therapy (except for subjects with breast cancer receiving it as adjuvant therapy).

Strong CYP3A4 inducers (including herbal supplements such as St. John's wort) unless approved by the principal investigator and medical monitor.

Known CYP3A4 and/or P-gp sensitive substrates with narrow therapeutic window, unless approved by the principal investigator and medical monitor.

CYP3A4 and/or UGT1A1 inhibitors (Part 1 Cohort B, Part 2 Cohort F and Cohort G only) unless approved by the principal investigator and medical monitor

### Other Investigational Agents

Anti-EGFR targeting agents other than panitumumab.

If a subject needs palliative radiotherapy or surgery for pain control during the course of the study, all study drugs should be withheld. A subject may be allowed to resume study drug after discussion with the principal investigator and medical monitor.

### Dose Limiting Toxicities:

The dose limiting toxicity (DLT) window (i.e., DLT-evaluable period) will be the first 28 days of sotorasib and panitumumab treatment (starting cycle 1, day 1). The grading of AEs will be based on the guidelines provided in the CTCAE version 5.0. A subject will be DLT evaluable if the subject has completed the DLT window as described above and received >80% of the planned dose of sotorasib and panitumumab within the first 28 days or experienced a DLT any time during the DLT window. DLT is defined as any adverse event meeting the criteria listed below occurring during the first treatment cycle and attributable to sotorasib and/or panitumumab.
(1) An adverse event that results in permanent discontinuation of any investigational product;
(2) Febrile neutropenia
(3) Neutropenic infection
(4) Grade 4 neutropenia of any duration
(5) Grade 3 neutropenia lasting >7 days
(6) Grade 3 thrombocytopenia for >7 days
(7) Grade 3 thrombocytopenia with grade: 2 bleeding
(8) Grade 4 thrombocytopenia
(9) Grade 4 anemia
(10) Grade 4, vomiting or diarrhea
(11) Grade 3 vomiting or grade 3 diarrhea lasting more than 3 days despite optimal medical support
(12) Grade ≥3 nausea lasting 3 days or more despite optimal medical support
(13) Grade 3 ALT or AST elevations lasting more than 5 days (only for subjects without liver metastasis at baseline)
(14) Grade 4 elevations of ALT or AST of any duration
(15) Grade ≥3 bilirubin elevation
(16) Any other grade ≥3 AE with the following exceptions:
   DLT Exemption: grade 3 fatigue <1 week
   DLT Exemption: grade 3 panitumumab skin toxicity
   DLT Exemption: Asymptomatic grade 3 electrolyte abnormalities that last <72 hours, are not clinically complicated, and resolve spontaneously or respond to medical interventions
   DLT Exemption: grade 3 amylase or lipase that is not associated with symptoms or clinical manifestations of pancreatitis
   DLT Exemption: Other select lab abnormalities that do not appear to be clinically relevant or harmful to the patient and/or can be corrected with replacement or modifications (e.g., grade 3 lymphopenia, grade 3 hypoalbuminemia, grade 3 hypomagnesemia)
   DLT Exemption: grade 3 infusion reaction
(17) Any subject meeting the criteria for Hy's Law case (i.e., severe drug-induced liver injury [DILI]) will be considered a DLT. A Hy's Law case is defined as: AST or ALT values of ≥3×ULN AND with serum total bilirubin level (TBL) of >2×ULN without signs of cholestasis and with no other clear alternative reason to explain the observed liver related laboratory abnormalities.

If a subject experiences a DLT during the DLT evaluation period, study treatment should be discontinued for that subject. However, if the investigator believes that the subject is benefiting clinically from the therapy, therapy may be resumed with the consideration of a dose reduction.

Sotorasib dose modification guidelines for hematologic and non-hematologic toxicities.

| | Recommended Action | |
|---|---|---|
| Toxicity | Hold Until: | Restart Dose: |
| Grade ≥3 nausea, vomiting, or | Recovery to grade 1 or | Resume dosing at 1 |
| diarrhea lasting longer than 3 days | less or to baseline grade | dose lower^{a} |
| despite optimal medical support | | |
| Suspected interstitial lung | ILD/pneumonitis confirmed | If confirmed, permanently |
| disease (ILD)/pneumonitis | or excluded | discontinue sotorasib |
| of any grade | | If excluded, restart at same |
| | | dose if no other sotorasib dose |
| | | modification guidelines are |
| | | applicable |
| Any other drug-related | Recovery to grade 1 or | Resume dosing at 1 dose |
| toxicity ≥ grade 3^{b} | less or to baseline grade | lower^{a} |

^{a}Subjects may be resumed at a dose lower than the recommended restarting dose after discussion with the Medical Monitor
^{b}For subjects with hepatotoxicity, see below

If sotorasib is held, panitumumab should be held as well.

Dose reduction levels of sotorasib for toxicity management of individual subjects is provided in the following table.

Sotorasib Doses (mg QD)

| Starting Dose | Dose −1 | Dose −2 |
|---|---|---|
| 960 | 480 | 240 |

QD = once daily

Hepatotoxicity Guidelines for Sotorasib: Guidelines for management and monitoring of subjects with increased AST, ALT, or alkaline phosphatase (ALP) are presented in the table below.

If the conditions for permanent discontinuation are met (below): Participant
to be permanently discontinued
AST or ALT > 3x ULN and INR > 1.5x ULN (for subjects not on anticoagulation
therapy) in the presence of no important alternative causes for
elevated AST/ALT values
OR
AST or ALT > 3x ULN and TBL > 2x ULN in the presence of no important alternative
causes for elevated AST/ALT and/or TBL values
If conditions are not met: Exclude other causes^{a}
Upon failure to identify any other causes and sotorasib relation to increase in
LFTs cannot be excluded, proceed with guidelines below:

| | Sotorasib | Medical | Monitoring and |
|---|---|---|---|
| CTCAE Grade | Action | Management | Follow-up |
| Grade 2 AST or ALT and ALP ≤ 8x | Continue | Consider | Closely monitor liver function tests |
| ULN with no clinical symptoms | | steroids^{b} | |
| consistent with hepatitis | | | |
| (right upper quadrant | | | |
| pain/tenderness, fever, nausea, | | | |
| vomiting, and jaundice) | | | |
| Grade 2 AST or ALT | First Occurrence | Initiate | Closely monitor liver function tests |
| with symptoms | Withhold | steroids^{b} | Await resolution to baseline or grade |
| Or | | | 1 and resolution or improvement of |
| Grade 3 or 4 | | | hepatitis symptoms |
| AST or ALT | | | Restart at 1 dose level reduction^{c, e} |
| Or | Second Occurrence | Initiate | Closely monitor liver function tests |
| 8x ULN ALP^{d} | Withhold | steroids^{b} | Await resolution to baseline or grade |
| | | | 1 and resolution or improvement of |
| | | | hepatitis symptoms |
| | | | Resume at an additional 1 dose level |
| | | | reduction only with MEDICAL |
| | | | MONITOR approval^{c, e} |
| | Third Occurrence | NOT APPLICABLE | |
| | Permanently | | |
| | discontinue | | |
| | Sotorasib | | |

ALP = alkaline phosphatase;
ALT = alanine aminotransferase;
AST = aspartate aminotransferase;
CTCAE = Common Terminology Criteria for Adverse Events;
INR = international normalized ratio;
LFT = liver function test;
TBL = total bilirubin;
ULN = upper limit of normal
^{a}If increase in AST/ALT is likely related to alternative agent, discontinue causative agent and await resolution to baseline or grade 1 prior to resuming sotorasib.
^{b}For example: prednisone 1 to 2 mg/kg/day, dexamethasone equivalent, or methylprednisone equivalent, followed by a taper. The taper may occur after restarting sotorasib.
^{c}Close monitoring at restart (e.g., daily LFTs × 2, then weekly × 4). Sotorasib dose may be increased after discussion with Medical Monitor.
^{d}There is no limit to the number of sotorasib re-challenges for isolated alkaline phosphatase elevations that resolve to baseline or grade 1.
^{e}Dose decrements below 240 mg are not allowable. Subjects may restart at same dose without dose reduction.

Hepatotoxicity Response: Subjects with abnormal hepatic laboratory values (i.e., alkaline phosphatase (ALP), aspartate aminotransferase (AST), alanine aminotransferase (ALT), total bilirubin (TBL)) and/or international normalized ratio (INR) and/or signs/symptoms of hepatitis (as described below) may meet the criteria for withholding or permanent discontinuation of sotorasib.

The following stopping and/or withholding rules apply to subjects for whom another cause of their changes in liver biomarkers (TBL, INR and transaminases) has not been identified. Important alternative causes for elevated AST/ALT and/or TBL values include, but are not limited to: Hepatobiliary tract disease; Viral hepatitis (e.g., hepatitis A/B/C/D/E, Epstein-Barr Virus, cytomegalovirus, herpes simplex virus, varicella, toxoplasmosis, and parvovirus); Right sided heart failure, hypotension or any cause of hypoxia to the liver causing ischemia; Exposure to hepatotoxic agents/drugs or hepatotoxins, including herbal and dietary supplements, plants and mushrooms; Heritable disorders causing impaired glucuronidation (e.g., Gilbert's syndrome, Crigler-Najjar syndrome) and drugs that inhibit bilirubin glucuronidation (e.g., indinavir, atazanavir); Alpha-one antitrypsin deficiency; Alcoholic hepatitis; Autoimmune hepatitis; Wilson's disease and hemochromatosis; Nonalcoholic fatty liver disease including steatohepatitis; and/or Non-hepatic causes (e.g., rhabdomyolysis, hemolysis).

Rechallenge may be considered if an alternative cause for impaired liver tests (ALT, AST, ALP) and/or elevated TBL, is discovered and/or the laboratory abnormalities resolve to normal or baseline, as described in the below.

| Analyte | Temporary Withholding | Permanent Discontinuation |
|---|---|---|
| TBL | >3 × ULN | >2 × ULN |
| | at any time | OR |
| INR | — | >1.5 × (for subjects not on |
| | OR | anticoagulation therapy) |
| | | AND |
| AST/ | >5 × ULN at any time | In the presence of no |
| ALT | >3 × ULN with clinical | important alternative |
| | signs or symptoms | causes for elevated |
| | that are consistent | AST/ALT and/or TBL |
| | with hepatitis (such as | values |
| | right upper quadrant | >3 × ULN |
| | pain/tenderness, fever, | (when baseline was <ULN) |
| | nausea, vomiting, and | |
| | jaundice) | |
| | OR | |
| ALP | >8 × ULN at any time | — |

ALP = alkaline phosphatase;
ALT = alanine aminotransferase;
AST = aspartate aminotransferase;
INR = international normalized ratio;
TBL = total bilirubin;
ULN = upper limit of normal

### Panitumumab Dose Modifications:

For subjects who experience toxicities while on study, 1 or more doses of panitumumab are withheld, reduced, or delayed (administered at >14 day intervals). Exemplary panitumumab dose reductions are listed in the table below.

| | Starting | First Dose | Second Dose |
|---|---|---|---|
| | Dose | Reduction | Reduction |
| Percentage (%) | 100 | 80 | 60 |
| mg/kg | 6 | 4.8 | 3.6 |

Exemplary panitumumab dose modification guidelines due to dermatological toxicities are provided in the table below.

| Occurrence | | | |
|---|---|---|---|
| of Skin | Administration | | |
| Symptom(s): | of | | |
| ≥grade 3 | Panitumumab | Outcome | Dose Regulation |
| Initial | Stop 1 or | Improved | Continue infusion at |
| occurrence | 2 doses | (<grade 3) | 100% of original dose |
| | | Not recovered | Discontinue |
| At the second | Stop 1 or | Improved | Continue infusion at |
| occurrence | 2 doses | (<grade 3) | 80% of original dose |
| | | Not recovered | Discontinue |
| At the third | Stop 1 or | Improved | Continue infusion at |
| occurrence | 2 doses | (<grade 3) | 60% of original dose |
| | | Not recovered | Discontinue |
| At the fourth | Discontinue | — | — |
| occurrence | | | |

In the event of severe or life-threatening inflammatory or infectious complications, consider withholding or discontinuing panitumumab as clinically appropriate.

It is recommended that patients wear sunscreen and hats and limit sun exposure while receiving panitumumab as sunlight can exacerbate any skin reactions that may occur. Proactive skin treatment including skin moisturizer, sunscreen (SPF >15 UVA and UVB), and topical steroid cream (not stronger than 1% hydrocortisone) may be useful in the management of skin toxicities. Subjects may be advised to apply moisturizer and sunscreen to face, hands, feet, neck, back and chest every morning during treatment, and to apply the topical steroid to face, hands, feet, neck, back and chest every night. Treatment of skin reactions should be based on severity and may include a moisturizer, sunscreen (SPF >15 UVA and UVB), and topical steroid cream (not stronger than 1% hydrocortisone) applied to affected areas, and/or oral antibiotics, as prescribed by a physician.

In the event of acute onset or worsening of pulmonary symptoms, consider withholding panitumumab. If interstitial lung disease is confirmed, discontinue panitumumab.

For toxicities other than dermatologic or pulmonary, withhold panitumumab for any grade 3 or 4 panitumumab-related toxicity with the following exceptions:
Panitumumab will only be withheld for symptomatic grade 3 or 4 hypomagnesemia and/or hypocalcemia that persists despite aggressive magnesium and/or calcium replacement
Panitumumab will only be withheld for grade 3 or 4 nausea, diarrhea, or vomiting that persists despite maximum supportive care

For toxicities other than dermatologic: If panitumumab was withheld, administration may recommence once the adverse event has improved to grade 1 or returned to baseline.

### Infusion Reactions:

Infusion reactions may manifest as fever, chills, dyspnea, bronchospasm, hypotension, or anaphylaxis.

Reduce infusion rate by 50% in patients experiencing a mild or moderate (grade 1 or 2) infusion reaction for the duration of that infusion.

Terminate the infusion in patients experiencing severe infusion reactions. Depending on the severity and/or persistence of the reaction, permanently discontinue panitumumab.

### FOLFIRI Dose Reductions

| | Starting | Dose | Dose | Dose |
|---|---|---|---|---|
| | Dose | Level-1 | Level-2 | Level-3 |
| Drug | (mg/m²) | (mg/m²) | (mg/m²) | (mg/m²) |
| Irinotecan | 180 | 150 | 120 | Discontinue |
| Leucovorin^{a} | 400 racemate | 400 racemate | 400 racemate | Discontinue |
| | leucovorin | leucovorin | leucovorin | |
| 5-FU bolus | 400 | 320 | 240 | Discontinue |
| 5-FU 46 to 48- | 2400 | 2000 | 1600 | Discontinue |
| hour infusion | | | | |

The dose of irinotecan, 5-fluorouracil, and leucovorin will be calculated based on height and weight on cycle 1 day 1 and should be recalculated using the current weight at each dose. If it is institutional policy, FOLFIRI dose recalculation is not required if the subject's weight changes by <10%. The reason for dose change of FOLFIRI is to be recorded on each subject's CRE(s).

### FOLFIRI Dose Modification Guidelines

Dose Modifications for the Next Cycle Based on the Worst Toxicity Observed

| | | |
|---|---|---|
| Toxicity Grade^{b} | 5-FU | Irinotecan |

Thrombocytopenia (since prior treatment)

| | | |
|---|---|---|
| 0 or 1 | Maintain dose level | Maintain dose level |
| 2 | Hold until recovery to grade 1 or | Maintain dose level |
| | less. Decrease 1 dose level | |
| 3 | Hold until recovery to grade 1 or | Hold until recovery to grade 1 or |
| | less. Decrease 1 dose level | less. Decrease 1 dose level |
| 4 | Hold until recovery to grade 1 or | Hold until recovery to grade 1 or |
| | less. Decrease 2 dose levels | less. Decrease 2 dose levels |

Neutropenia and leukopenia (since prior treatment)

| | | |
|---|---|---|
| 0 or 1 | Maintain dose level | Maintain dose level |
| 2 | Decrease 1 dose level | Maintain dose level |
| 3 | Hold until recovery to grade 1 or | Hold until recovery to grade 1 or |
| | less. Decrease 1 dose level | less. Decrease 1 dose level |
| 4 | Hold until recovery to grade 1 or | Hold until recovery to grade 1 or |
| | less. Decrease 2 dose levels | less. Decrease 2 dose levels |
| Neutropenic fever or infection^{c} | Hold until recovery to grade 1 or | Hold until recovery to grade 1 or |
| | less. Decrease 2 dose levels | less. Decrease 2 dose levels |

Diarrhea^{d} (since prior treatment)

| | | |
|---|---|---|
| 0 or 1 | Maintain dose level | Maintain dose level |
| 2 | If maximal anti-diarrheals were | If maximal anti-diarrheals were |
| | used, hold until recovery to grade 1 | used, hold until recovery to grade 1 |
| | or less or baseline. If maximal | or less or baseline. If maximal |
| | antidiarrheals were not used, | antidiarrheals were not used, |
| | investigator judgement will | investigator judgement will |
| | determine if dose hold is needed. | determine if dose hold is needed. |
| | Decrease 1 dose level | Maintain dose level |
| 3 | Hold until recovery to grade 1 or | Hold until recovery to grade 1 or |
| | less or baseline. Decrease 1 dose | less or baseline. Decrease 1 dose |
| | level | level |
| 4 | Hold until recovery to grade 1 or | Hold until recovery to grade 1 or |
| | less or baseline. Decrease 2 dose | less or baseline. Decrease 2 dose |
| | levels | levels |

Mucositis/Stomatitis (since prior treatment)

| | | |
|---|---|---|
| 0, 1, or 2 | Maintain dose level | Maintain dose level |
| 3 | Hold until recovery to grade 1 or | Hold until recovery to grade 1 or |
| | less or baseline. Decrease 1 dose | less or baseline. Maintain dose |
| | level | level |
| 4 | Hold until recovery to baseline. | Hold until recovery to grade 1 or |
| | Decrease 2 dose levels | less or baseline. Maintain dose |
| | | level |

Vomiting (since prior treatment)

| | | |
|---|---|---|
| 0, 1, or 2 | Maintain dose level | Maintain dose level |
| 3 | Hold until recovery to grade 1 or | Hold until recovery to grade 1 or |
| | less or baseline. Maintain dose | less or baseline. Decrease 1 dose |
| | level | level |
| 4 | Hold until recovery to grade 1 or | Hold until recovery to grade 1 or |
| | less or baseline. Decrease 1 dose | less or baseline. Decrease 1 dose |
| | level | level |

Palmar-plantar erythrodyesthesia

| | | |
|---|---|---|
| 0 or 1 | Maintain dose level | Maintain dose level |
| 2 | Hold until recovery to grade 1 or | Hold until recovery to grade 1 or |
| | less or baseline. Maintain dose | less or baseline. Maintain dose |
| | level | level |
| 3 | Hold until recovery to grade 1 or | Hold until recovery to grade 1 or |
| | less or baseline. Decrease 1 dose | less or baseline. Maintain dose |
| | level | level |

Other nonhematologic toxicities (except alopecia and skin-related toxicities)^{e}

| | | |
|---|---|---|
| 0, 1, or 2 | Maintain dose level | Maintain dose level |
| 3 | Hold until recovery to grade 1 or | Hold until recovery to grade 1 or |
| | less or baseline. Decrease 1 dose | less or baseline. Decrease 1 dose |
| | level | level |
| 4 | Hold until recovery to grade 1 or | Hold until recovery to grade 1 or |
| | less or baseline. Decrease 2 dose | less or baseline. Decrease 2 dose |
| | levels | levels |

IV = intravenous; NCI CTCAE = National Cancer Institute Common Terminology Criteria for Adverse Events
^{a} There will be no leucovorin dose reduction.
^{b}NCI CTCAE (Version 5.0)
^{c}Absolute neutrophil count <1000/mm³ and temperature ≥38.5° C.
^{d}Despite maximum supportive care.
^{e}Bilirubin grade 3 and 4 hold dose; once the bilirubin level has resolved to ≤grade 1, a dose reduction for 5-FU and irinotecan is required.

### Radiological Imaging Assessment

The extent of disease will be evaluated by contrast-enhanced MRI/CT according to RECIST v1.1. In order to reduce radiation exposure for subjects, the lowest dose possible should be utilized whenever possible.

The screening scans must be performed within 28 days prior to enrollment and will be used as baseline. Imaging performed as part of standard of care that falls within the screening window given for scans may be used for the baseline scan as long as it meets the scan requirements for screening. All subsequent scans will be performed in the same manner as at screening, with the same contrast, preferably on the same scanner. Radiological assessment must include CT of the chest, and contrast-enhanced CT or MRI of the abdomen and pelvis, as well as assessment of all other known sites of disease as detailed within the Site Imaging Manual.

The same imaging modality, MRI field strength and IV and oral contrast agents used at screening should be used for all subsequent assessments. Liver specific MRI contrast agents should not be used. To reduce potential safety concerns, macrocyclic gadolinium contrast agents are recommended per National Health Institute guidelines or follow local standards if more rigorous.

During treatment and follow-up, radiological imaging of the chest, abdomen, pelvis, as well as all other known sites of disease, will be performed independent of treatment cycle every 6±1 weeks for the first 4 response assessments. After 4 (6 week) response assessments, radiological imaging and tumor assessment will be performed every 12±1 weeks. Radiologic imaging and tumor assessment will be performed until disease progression, start of new anti-cancer treatment, death, withdrawal of consent or until end of study. Imaging may also be performed more frequently if clinically necessitated at the discretion of the managing physician. Radiographic response (CR, PR) requires confirmation by a repeat, consecutive scan at least 4 weeks after the first documentation of response and may be delayed until the next scheduled scan to avoid unnecessary procedures.

All NSCLC subjects, subjects with a history of brain metastases, and subjects with signs and symptoms suggestive of brain metastases must have MRI of the brain performed within 28 days prior to first dose of sotorasib. Subsequently, brain scans may be performed at any time if needed, in the judgement of the managing physician. All brain scans on protocol are required to be MRI unless MRI is contraindicated, and then CT with contrast is acceptable.

Radiological imaging assessment at the end of the study or during the end of treatment (EOT) visit should be performed only for subjects that discontinue treatment for a reason other than disease progression per RECIST v1.1 guidelines.

Determination of disease response for clinical management of subjects will be assessed at the clinical sites per RECIST v1.1. Scans may be submitted to a central imaging core laboratory for archival and (if necessary) independent response assessment utilizing RECIST v1.1 criteria. Exploratory imaging analyses may be performed centrally and may include tumor volumetrics, viable tumor measurements, tissue necrosis ratios, and lesion texture analysis (radiomics).

Response Evaluation Criteria in Solid Tumors Version 1.1 (RECIST 1.1) Definitions

### Measurable Lesions

Measurable Tumor Lesions—Non-nodal lesions with clear borders that can be accurately measured in at least 1 dimension with longest diameter 10 mm in CT/MRI scan with slice thickness no greater than 5 mm. When slice thickness is greater than 5 mm, the minimum size of measurable lesion should be twice the slice thickness.

Nodal Lesions—Lymph nodes are to be considered pathologically enlarged and measurable, a lymph node must be 15 mm in short axis when assessed by CT/MRI (scan slice thickness recommended to be no greater than 5 mm). At baseline and in follow-up, only the short axis is measured and followed. Nodal size is normally reported as two dimensions in the axial plane. The smaller of these measures is the short axis (perpendicular to the longest axis).

Irradiated Lesions—Tumor lesions situated in a previously irradiated area, or in an area subjected to other loco-regional therapy, are not measurable unless there has been demonstrated progression in the lesion prior to enrollment.

Non-measurable Lesions: All other lesions, including small lesions (longest diameter <10 mm or pathological lymph nodes with 10 mm but to <15 mm short axis with CT scan slice thickness no greater than 5 mm) are considered non-measurable and characterized as non-target lesions.

Other examples of non-measurable lesions include: Lesions with prior local treatment: tumor lesions situated in a previously irradiated area, or an area subject to other loco-regional therapy, should not be considered measurable unless there has been demonstrated progression in the lesion; Biopsied lesions; Categorically, clusters of small lesions, bone lesions, inflammatory breast disease, and leptomeningeal disease are non-measurable.

### Methods of Measurement

Measurement of Lesions—The longest diameter of selected lesions should be measured in the plane in which the images were acquired (axial plane). All measurements should be taken and recorded in metric notation. All baseline evaluations should be performed as closely as possible to the beginning of treatment and not more than 4 weeks before study Day 1.

Methods of Assessment—The same method of assessment and the same technique should be used to characterize each identified and reported lesion throughout the trial.

CT/MRI—Contrast-enhanced CT or MRI should be used to assess all lesions. Optimal visualization and measurement of metastasis in solid tumors requires consistent administration (dose and rate) of IV contrast as well as timing of scanning. CT and MRI should be performed with <5 mm thick contiguous slices.

### Baseline Documentation of “Target” and “Non-Target” Lesions

Target Lesions—All measurable lesions up to a maximum of two (2) lesions per organ and five (5) lesions in total, representative of all involved organs should be identified as target lesions and recorded and measured at baseline.
Target lesions should be selected on the basis of their size (lesions with the longest diameter) and suitability for accurate repeated measurements.
Pathologic lymph nodes (with short axis ≥15 mm) may be identified as target lesions. All other pathological nodes (those with short axis ≥10 mm but <15 mm) should be considered non-target lesions.
A sum of the diameters (longest for non-nodal lesions, short axis for nodal lesions) for all target lesions are calculated and reported as the baseline sum of diameters. The baseline sum of diameters are used as reference by which to characterize objective tumor response.

Non-Target Lesions—All other lesions (or sites of disease) including pathological lymph nodes should be identified as non-target lesions and should also be recorded at baseline. Measurements of these lesions are not required, and these lesions should be followed as “present”, “absent”, or “unequivocal progression” throughout the study. In addition, it is possible to record multiple non-target lesions involving the same organ as a single item on the case report form (e.g., “multiple enlarged pelvic lymph nodes” or “multiple liver metastases”).

### Response Criteria

Evaluation of Target Lesions

| | |
|---|---|
| * Complete | Disappearance of all target lesions. Any pathological |
| Response (CR): | lymph nodes (whether target or non-target) must |
| | have reduction in short axis to <10 mm. |
| * Partial | At least a 30% decrease in the sum of the diameters |
| Response (PR): | of target lesions, taking as reference the baseline |
| | sum of diameters. |
| * Progressive | At least a relative 20% increase and an absolute |
| Disease (PD): | increase of 5 mm in the sum of the diameters of |
| | target lesions, taking as reference the smallest |
| | sum on study, or the appearance of 1 or more |
| | new lesions. |
| * Stable | Neither sufficient shrinkage to qualify for PR |
| Disease (SD) | nor sufficient increase to qualify for PD, taking |
| | as reference the smallest sum of diameters |
| | since the treatment started. |

Evaluation of Non-target Lesions

| | |
|---|---|
| * Complete | Disappearance of all non-target lesions and |
| Response (CR): | normalization of tumor marker levels. All |
| | lymph nodes must be non-pathological in |
| | size (<10 mm short axis). |
| * Incomplete | Persistence of one or more non-target lesion(s) |
| Response/Stable | or/and maintenance of tumor marker levels |
| Disease (SD): | above the normal limits. |
| * Progressive | Unequivocal progression of existing non-target |
| Disease (PD) | lesions and/or appearance of one or more |
| | new lesions.¹ |

¹To achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of 1 or more non-target lesions is usually not sufficient to qualify for unequivocal progression status.

### Evaluation of Overall Response

The best overall response is the best response recorded from the start of the study treatment until the end of treatment or disease progression/recurrence (taking as reference for PD the smallest measurements recorded since the treatment started).

In general, the subject's best response assignment depends on the findings of both target and non-target disease and also take into consideration the appearance of new lesions.

Time Point response: Subjects with Target (+/− Non-target) Disease

| | Non-target | New | Overall |
|---|---|---|---|
| Target Lesions | Lesions | Lesions | Response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |

NE = Not evaluable

Time Point response: Subjects with Non-target Disease

| Non-Target Lesions | New Lesions | Overall Response |
|---|---|---|
| CR | No | CR |
| Non-CR/non-PD | No | Non-CR/non-PD¹ |
| Not all evaluated | No | NE |
| Unequivocal PD | Yes or No | PD |
| Any | Yes | PD |

¹“Non-CR/non-PD” is preferred over “SD” for non-target disease since SD is increasingly used as endpoint for assessment of efficacy in some trials so as to assign this category when no lesions can be measured is not advised.

Overall Response: Confirmation of Complete
Response (CR) and Partial Response (PR) required

| Overall | Overall | |
|---|---|---|
| Response | Response | |
| First | Second | |
| Time Point | Time Point | Best Overall Response |
| CR | CR | CR |
| CR | PR | SD, PD, or PR¹ |
| CR | SD | SD provided minimum criteria for |
| | | SD duration met, otherwise, PD |
| CR | PD | SD provided minimum criteria for |
| | | SD duration met, otherwise, PD |
| CR | NE | SD provided minimum criteria for |
| | | SD duration met, otherwise, NE |
| PR | CR | PR |
| PR | PR | PR |
| PR | SD | SD |
| PR | PD | SD provided minimum criteria for |
| | | SD duration met, otherwise, PD |
| PR | NE | SD provided minimum criteria for |
| | | SD duration met, otherwise, NE |
| NE | NE | NE |

¹If a CR is truly met at first time point, then any disease at a subsequent time point, even if disease meeting PR criteria relative to baseline, makes the disease PD at that point (since disease must have reappeared after CR). Best response would depend upon whether minimum duration for SD was met. However, sometimes “CR” may be claimed when subsequent scans suggest small lesions were likely still present and in fact the subject had PR, not CR at the first time point. Under these circumstances, the original CR should be changed to PR and the best response is PR.

### Special Notes on Response Assessment

Nodal lesions—Lymph nodes identified as target lesions should always have the actual short axis measurement recorded, even if the nodes regress to below 10 mm on study. In order to qualify for CR, each node must achieve a short axis <10 mm, NOT total disappearance. Nodal target lesion short axis measurements are added together with target lesion' longest diameter measurements to create the sum of target lesion diameters for a particular assessment (time point).

Target lesions that become “too small to measure”—While on study, all lesions (nodal and non-nodal) recorded at baseline should have their measurements recorded at each subsequent evaluation. If a lesion becomes less than 5 mm, the accuracy of the measurement becomes reduced. Therefore, lesions less than 5 mm are considered as being “too small to measure”, and are not measured. With this designation, they are assigned a default measurement of 5 mm. No lesion measurement less than 5 mm should be recorded, unless a lesion totally disappears and “0” can be recorded for the measurement.

New lesions—The term “new lesion” always refers to the presence of a new finding that is definitely tumor. New findings that only may be tumor, but may be benign (infection, inflammation, etc.) are not selected as new lesions, until that time when the review is certain they represent tumor.
If a new lesion is equivocal, for example because of its small size, continued therapy and follow-up evaluation will clarify if it represents truly new disease. If repeat scans confirm there is definitely a new lesion, then progression should be declared using the date of the initial scan.
A lesion identified on a follow-up study in an anatomical location that was not scanned at baseline is considered a new lesion and will indicate disease progression, regardless of any response that may be seen in target or non-target lesions present from baseline.

Subjects with a global deterioration of health status requiring discontinuation of treatment without objective evidence of disease progression at that time should be classified as having “symptomatic deterioration.” Every effort should be made to document the objective progression with an additional imaging assessment even after discontinuation of treatment.

In some circumstances it may be difficult to distinguish residual disease from scar or normal tissue. When the evaluation of complete response (CR) depends on this determination, it is recommended that the residual lesion be further investigated by fluorodeoxyglucose-positron emission tomography (FDG-PET) or PET/computed tomography (PET/CT), or possibly fine needle aspirate/biopsy, to confirm the CR status.

### Confirmation Measurement/Duration of Response

Response Confirmation—In non-randomized trials where response is the primary endpoint, confirmation of PR and CR is required to ensure responses identified are not the result of measurement error.

Duration of overall response—The duration of overall response is measured from the time measurement criteria are first met for CR/PR (whichever is first recorded) until the first date the recurrent or progressive disease is objectively documented or death, whichever is earlier.

Duration of Stable Disease—SD is measured from the start of the treatment until the criteria for disease progression are met, taking as reference the smallest measurements recorded since the treatment started, or death, whichever is earlier.

### ECOG Performance and NYHA Classification

ECOG Performance Status Scale

| Grade | Descriptions |
|---|---|
| 0 | Fully active, able to carry on all pre-disease performance |
| | without restriction. |
| 1 | Restricted in physically strenuous activity, but ambulatory |
| | and able to carry out work of a light or sedentary nature |
| | (e.g., light housework, office work). |
| 2 | Ambulatory and capable of all self-care, but unable to |
| | carry out any work activities. Up an dabout more than |
| | 50% of waking hours. |
| 3 | Capable of only limited self-care, confined to bed or |
| | chair more than 50% of waking hours. |
| 4 | Completely disabled. Cannot carry on any self-care. |
| | Totally confined to bed or chair. |
| 5 | Dead. |

Source: Oken et al, 1982; [0001] ECOG = Eastern Cooperative Oncology Group;

### New York Heart Association Functional Classification

Class I No limitation of physical activity. Ordinary physical activity does not cause undue fatigue, palpitation or dyspnea.

Class II Slight limitation of physical activity. Comfortable at rest, but ordinary physical activity results in fatigue, palpitation or dyspnea.

Class III Marked limitation of physical activity. Comfortable at rest, but less than ordinary activity causes fatigue, palpitation or dyspnea.

Class IV Unable to carry out any physical activity without discomfort. Symptoms of cardiac insufficiency may be present even at rest. If any physical activity is undertaken, discomfort is increased.

### Preliminary Results (Apr. 23, 2021 Data Cut):

8 patients (5 female, 3 male, median age: 60.5 yrs [range: 31-79]) were enrolled in dose exploration with 960 mg QD sotorasib and 6 mg/kg IV Q2W panitumumab. Median number of lines of therapy for metastatic disease was 3.5 (range 1-10); 5 pts had prior sotorasib treatment. Median treatment (tx) duration was 4.4 months (range: 1.4, 8.8). No dose limiting toxicities (DLTs) were observed during the DLT evaluation period (first 28 days). Tx-related adverse events (TRAEs) of any grade related to sotorasib or panitumumab were reported for 4 and 8 patients, respectively. No grade 4 or fatal TRAEs occurred. Two patients had panitumumab TRAEs leading to dose modification of panitumumab (1-dermatitis acneiform, 1-dry skin, rash, hypokalemia, hypomagnesemia) and 1 patient had a sotorasib TRAE leading to dose modification of sotorasib (diarrhea). There was 1 confirmed partial response, 5 stable disease (SD), 1 progressive disease (PD), and 1 not evaluated but with clinical PD. Of patients with prior sotorasib treatment, 4 had decrease in sum of target lesions; 4 had SD and 1 with PD developed new lesions despite a decrease in size of target lesions. Sotorasib exposures were similar to those observed in monotherapy study.

Results showed that the combination of sotorasib (960 mg QD) and panitumumab (6 mg/kg IV Q2W) was safe and tolerable with promising efficacy in heavily pretreated patients with KRAS G12C mutated CRC. Adverse events were consistent with the known adverse events for sotorasib and panitumumab. See also, Fakih et al., 2021 (Abstract #3245)

### Additional Preliminary Results (Aug. 6, 2021 Data Cut):

31 patients (21 females, 10 males, median age 58 years range 31-79) were enrolled in dose exploration with 960 mg QD sotorasib and 6 mg/kg IV Q2W panitumumab (Part 1 and Part 2 Combined Cohort A). Median number of lines of therapy for metastatic disease was 2; five patients (16.1%) had prior sotorasib therapy. Median treatment duration was 10.3 weeks (range 2.1-48.1 weeks) at the time of data cutoff. No dose limiting toxicities (DLTs) were observed during the DLT evaluation period (first 28 days). Treatment-related adverse events (TRAEs) of any grade were reported for 23 (74.2%) patients (for 14 (45.2%) patients related to sotorasib and for 23 (74.2%) related to panitumumab). No grade 4 or fatal TRAEs occurred. Four patients (12.9%) experienced Grade 3 TRAEs. Of those Grade 3 TRAEs, one patient experienced grade 3 hypokalemia, hypomagnesemia, dry skin, and rash (panitumumab-related), and panitumumab dose was modified; one patient experienced grade 3 dermatitis acneiform and myalgia (panitumumab-related), and panitumumab dose was modified only for dermatitis acneiform; one patient experienced grade 3 diarrhea (sotorasib-related), and sotorasib dose modified; one patient experienced grade 3 cellulitis, edema peripheral, and dermatitis acneiform (panitumumab-related), no dose change for either sotorasib or panitumumab). Of the TRAEs resulting in dose modification, 3 patients (9.7%) exhibited TRAEs (diarrheas, fatigue and hypokalemia) and sotorasib dose was modified; and 2 patients (6.5%) exhibited TRAEs (dermatitis acneiform and dry skin/rash/hypokalemia/hypomagnesemia) and panitumumab dose was modified. Sotorasib in combination with panitumumab was well tolerated, with no fatal TRAEs.

Observed tumor response is provided in the table below:

| | Part 1 Cohort A (N = 8) | | Part 2 Cohort A (N = 18) | | Part 1 + Part 2 | |
|---|---|---|---|---|---|---|
| Tumor response assessed | Sotorasib (960 mg) and | | Sotorasib (960 mg) and | | Combined Cohort | |
| by central review | panitumumab (6 mg/kg) | | panitumumab (6 mg/kg) | | (N = 26)* | |
| Disease control rate, n (%) | 6 | (75.0) | 15 | (83.3) | 21 | (80.8) |
| ORR, % (95% CI) | | | | | | |
| Confirmed: | 12.5 | (0.3, 52.7) | 16.7 | (3.6, 41.4) | 15.4 | |
| Confirmed and Unconfirmed | 12.5 | (0.3, 52.7) | 33.3 | (13.3, 59.0) | 26.9 | |
| Partial response (PR), n (%) | | | | | | |
| Confirmed | 1 | (12.5) | 3 | (16.7) | 4 | (15.4) |
| Confirmed and Unconfirmed | 1 | (12.5) | 6 | (33.3) | 7 | (26.9) |
| Stable disease (SD)** , n (%) | 5 | (62.5) | 12 | (66.7) | 17 | (65.4) |
| Progressive disease (PD), n (%) | 1 | (12.5) | 2 | (11.1) | 3 | (11.5) |
| Not done, n (%) | 1 | (12.5) | 1 | (5.6) | 2 | (7.7) |

*Efficacy analysis set includes all patients who received ≥1 dose of investigational products, have ≥1 measurable lesions at baseline assessment using RECIST 1.1, and have the opportunity to be followed for ≥7 weeks starting from day 1.
**includes patients with unconfirmed partial response, awaiting confirmatory scan. ORR, objective response rate

Overall, 27% of the patients (7 of 26) achieved response (including unconfirmed response, awaiting confirmation) and 81% of the patients (21 of 26) achieved disease control. For Part 1, Cohort A, 5 of 8 patients in dose exploration had prior KRAS^{G12C} inhibitor treatment; nonetheless, the majority of all patients (75%, 6 of 8 patients) experienced a decrease in target lesion size (−14.5% to −100.0%). The majority of patients (80%, 4 of 5 patients) with prior KRAS^{G12C} inhibitor exposure had a best response of stable disease. Four of the 5 patients with prior KRAS^{G12C} inhibitor treatment showed between 14.5% and 30.3% reduction in target lesion size. One patient (KRAS^{G12C} inhibitor naïve) achieved a PR within two months of treatment and remains on treatment at the time of data cutoff. For the responder in this cohort, a 100% reduction in target lesion size was observed.

For Part 2, Cohort A (n=18), overall, 83% of patients remain on treatment, with 2 patients remaining on treatment after 6 months. A decrease in target lesion size (−2.4% to −61.8%) was observed in the majority (83%, 15 of 18 patients) of chemotherapy refractory metastatic colorectal cancer (mCRC) population treated in dose expansion (Part 2, Cohort A, n=18). This decrease appears to be durable. Mean time for progression-free survival cannot yet be determined.

Sotorasib exposures for patients administered both sotorasib and panitumumab were similar to those of patients administered sotorasib alone (in CodeBreaK 100 trial NCT03600883):
At day 1: tₘₐₓ (h) 1.0 for combination therapy, 1.9 for monotherapy;
   Cₘₐₓ (μg/mL) 8.01 for combination therapy, 9.71 for monotherapy;
   AUC₀₋₂₄ₕ (h*μg/mL) 77.2 for combination therapy, 103 for monotherapy
At day 8: tₘₐₓ (h) 1.0 for combination therapy, 2.0 for monotherapy;
   Cₘₐₓ (μg/mL) 7.50 for combination therapy, 6.50 for monotherapy;
   AUC₀₋₂₄ₕ (h*μg/mL) 51.7 for combination therapy, 50.3 for monotherapy

Collectively, results showed that the combination of sotorasib (960 mg QD) and panitumumab (6 mg/kg IV Q2W) was safe and tolerable with promising efficacy in chemorefractory patients with KRAS G12C mutated CRC. Adverse events were consistent with the known adverse events for sotorasib and panitumumab. The response rates of the combination of sotorasib and panitumumab were: 15.4% confirmed ORR and 26.9% ORR (including unconfirmed response awaiting confirmation). These ORRs were numerically higher than sotorasib monotherapy in KRAS G12C-mutated CRC (7.1% ORR) (Hong et al., 2020). Sotorasib exposures were similar to those observed in the monotherapy study. Sotorasib in combination with panitumumab is associated with signals of early promising efficacy in patients with KRAS.G12C-mutated CRC. See also, Fakih et al., 2021 (ePoster #3245).

### Additional Preliminary Results (Jun. 24, 2022 Data Cut-Off):

40 patients (30 females, 10 males, median age 58 years) were enrolled in dose exploration with 960 mg QD sotorasib and 6 mg/kg IV Q2W panitumumab (Part 2 Cohort A). Preliminary safety data on 23 of these patients and efficacy data on 18 of these patients was included in the Aug. 6, 2021, data cut reported above. Median number of lines of therapy for metastatic disease was 2; seven patients (18%) had prior regorafenib therapy and 7 patients (18%) had prior trifluridine/tipiracil therapy. (one patient had both regorafenib as a third-line therapy and trifluridine/tipiracil as a fourth-line therapy).

### Safety and Tolerability

Treatment-related adverse events (TRAEs) of any grade were reported for 37 (93%) patients (for 26 (65%) patients related to sotorasib and for 37 (93%) patients related to panitumumab). No grade 4 or fatal TRAEs occurred. Nine patients (23%) experienced Grade 3 TRAEs. Grade 3 TRAEs included rash (n=2, 5%), anaemia, fatigue, peripheral oedema, cellulitis, pustular rash, salmonellosis, skin infection, hypomagnesemia, malignant neoplasm progression, pulmonary embolism, dermatitis acneiform, and pruritus (n=1 patient each, 3%). Of the TRAEs resulting in dose modification, 6 patients (15%) exhibited TRAEs (pruritus, rash, anemia, diarrhea, hypokalemia) and sotorasib dose was modified; and 10 patients (25%) exhibited TRAEs (dermatitis acneiform, rash, dry skin, conjunctivitis, diarrhea, hypomagnesemia, paronychia, pruritus, rash pustular, vision blurred) and panitumumab dose was modified. Sotorasib in combination with panitumumab was well tolerated, with no fatal or Grade 4 TRAEs. No discontinuation of either drug was required.

### Pharmacokinetics

Sotorasib exposures for patients administered both sotorasib and panitumumab (n=35) were similar to those of patients administered sotorasib alone (n=32) (in CodeBreaK 100 trial NCT03600883):
tₘₐₓ (h), median (range): 1.0 (1.0-6.0) for combination therapy, 2.0 (0.3-6.0) for monotherapy;
Cₘₐₓ (μg/mL), mean (CV %): 9.64 (55%) for combination therapy, 7.50 (98%) for monotherapy;
AUC₀₋₂₄ₕ (h*μg/mL), mean (CV %): 65.8 (56%) for combination therapy, 65.3 (82%) for monotherapy
   Values are reported to 3 significant figures except for tₘₐₓ and CV %, which are presented as 2 significant figures and the nearest integer, respectively. AUC₀₋₂₄ₕ, area under the concentration-time curve from time 0 to 24 hours postdose; Cₘₐₓ, maximum observed drug concentration; tₘₐₓ, time to reach Cₘₐₓ; CV, coefficient of variation.

### Efficacy:

| Response by investigator | N = 40 |
|---|---|
| assessment | n (%) |
| ORR confirmed | 12 (30) |
| (95% CI) | (16.6, 46.5) |
| Complete response | 0 |
| Partial response | 12 (30) |
| Stable disease* | 25 (63) |
| Progressive disease | 3 (8) |
| DCR | 37 (93) |
| (95% CI) | (79.6, 98.4) |

*Minimum requirement for stable disease was 5 weeks.

A 30% confirmed response rate (ORR) for sotorasib+panitumumab in patients with chemorefractory mCRC was observed with a disease control rate (DCR) of 93%. An ORR subgroup analysis by primary tumor location (left v. right) was conducted. No obvious differences in response based on tumor location were noted (left (n=27, 30% ORR); right (n=13; 31% ORR)).

A reduction in RECIST target lesions was observed in 88% of patients. The median (range) duration of treatment was 5.9 (0.5, 11.3) months, with 25% of patients remaining on treatment at the time of data cutoff. The median duration of response was 4.4 months (range, 2.8-7.4 months).

With a median follow-up of 11.0 months, the median PFS was 5.7 months (see table below).

| Kaplan-Meier estimate of PFS | N = 40 |
|---|---|
| Median PFS, months (95% CI) | 5.7 (4.2, 7.6) |
| Left primary tumor | 5.8 (4.2, 7.8) |
| Right primary tumor | 5.5 (3.9, 8.2) |
| PFS rate, % (95% CI) | |
| At 3 months | 81.7 (65.4, 90.9) |
| At 6 months | 41.1 (24.7, 56.7) |
| At 9 months | 12.3 (3.4, 27.2) |

With a median follow-up of 8.8 months, the median OS was not yet reached (95% Cl: 10.4, NE) (see table below).

| Kaplan-Meier estimate of OS | N = 40 | |
|---|---|---|
| Median OS, months (95% CI) | NE | (10.4, NE) |
| Left primary tumor | NE | (10.4, NE) |
| Right primary tumor | NE | (8.7, NE) |
| OS rate, % (95% CI) | | |
| At 3 months | 97.5 | (83.6, 99.6) |
| At 6 months | 91.5 | (75.7, 97.2) |
| At 9 months | 82.5 | (61.8, 92.6) |

Collectively, results showed that the combination of sotorasib (960 mg QD) and panitumumab (6 mg/kg IV Q2W) was safe and tolerable with promising efficacy in chemorefractory patients with KRAS G12C mutated CRC. Adverse events were consistent with the known adverse events for sotorasib and panitumumab. The confirmed 30% ORR is 3-fold higher than previously reported with sotorasib monotherapy in KRAS G12C-mutated CRC (7.1% ORR) (Hong et al., 2020), with a DCR of 93%. No apparent difference based on tumor location was observed. Sotorasib exposures were similar to those observed in the monotherapy study. The median PFS of 5.7 months appears clinically meaningful and longer than that reported for sotorasib monotherapy (median PFS: 4.0 months, Hong et al., 2020), and OS data appear to be promising. See also Kuboki et al., 2022.

Additional Preliminary results on the triple combination (sotorasib, panitumumab and FOLFIRI) (Data cutoff Jun. 9, 2022):

As previously noted, Study 20190135 is the sotorasib protocol exploring sotorasib in combination with other anti-cancer therapies. Subprotocol H of this protocol explores the combination of sotorasib and panitumumab and the combination of sotorasib, panitumumab, and FOLFIRI. Part 1 Cohort B of Subprotocol H is the dose finding cohort of the combination of sotorasib, panitumumab, and FOLFIRI. A data snapshot was taken on Jun. 9, 2022. Six subjects were enrolled onto Dose Level One (sotorasib 960 mg oral daily, panitumumab 6 mg/kg intravenous every two weeks, FOLFIRI chemotherapy every 2 weeks) in this cohort, and no dose limiting toxicities (DLTs) were identified during the 28 day DLT evaluation window. These six subjects had a median of 3 prior lines of anti-cancer therapy (range 1-5), with 5 subjects having received prior irinotecan chemotherapy and all six subjects having received prior fluoropyrimidine chemotherapy. The objective response rate was 50% (3 of 6 subjects had confirmed partial response) and 100% disease control rate (subjects with complete response, partial response, or stable disease as best response). While subject numbers were low, the objective response rate of 50% in this heavily pretreated population with a median of 3 prior lines of therapy is very promising and compares favorably to the response rate of 10% observed with sotorasib monotherapy in study 20170543 Phase 2 CRC cohort and 30% observed with the combination of sotorasib and panitumumab in study 20190135 Subprotocol H Part 2 Cohort A. Dose Level One was declared the recommended phase 2 dose, and enrollment into Part 2 expansion cohorts are ongoing with subjects enrolling onto Part 2 Cohort F (treatment-naïve patients with metastatic colorectal cancer with KRAS G12C mutation) and Part 2 Cohort G (previously treated patients with metastatic colorectal cancer with KRAS G12C mutation).

### Example 2—Sotorasib in Combination with Panitumumab Vs Trifluridine and Tipiracil or Regorafenib

This is a phase 3, multicenter, randomized, open label, active-controlled study to evaluate efficacy and safety of two different doses of sotorasib and panitumumab vs (1) trifluridine and tipiracil or (2) regorafenib in previously treated metastatic CRC subjects with KRAS G12C mutation. The study will be conducted at approximately 100 sites. The study will consist of a screening period, a treatment period, a safety follow up and long term follow up period. Approximately 153 previously treated metastatic CRC subjects with KRAS G12C mutation will be enrolled and randomized 1:1:1 to receive either sotorasib 240 mg QD and panitumumab or sotorasib 960 mg QD and panitumumab or investigator's choice ((1) trifluridine and tipiracil; or (2) regorafenib). Investigator's choice will need to be declared prior to randomization. This trial is currently enrolling (Study 20190172, https://clinicaltrials.gov/ct2/show/NCT05198934; CodeBreaK 300).

Subjects will be stratified by prior anti-angiogenic therapy (Y vs N), time from initial diagnosis of metastatic disease to randomization (≥18 months, <18 months), and ECOG status (0 or 1 vs 2).

Cycle 1 Day 1 will be defined as the first day subject receives study medication; Tumor assessment will be conducted (by MRI and/or CT) at baseline, at 8 week (±7 days) intervals until blinded independent central review (BICR) assessed progression, start of another anti-cancer therapy, withdrawal of consent, loss to follow-up, or death, whichever occurs earliest. Safety follow-up CT/MRI should be performed only for subjects that discontinue treatment for a reason other than disease progression per RECIST 1.1 and has not had radiographic imaging performed within 8 weeks (±7 days of the visit). For subjects who do not have BICR assessed progression, radiographic assessments should continue in LTFU every 8 weeks (±7 days) until BICR assessed progression, start of another anticancer therapy, withdrawal of consent, loss to follow-up, or death, whichever occurs earliest. Tumor assessment and response will be determined by BICR using RECIST 1.1. Subjects on investigator's choice will be allowed to cross over to sotorasib and panitumumab after the primary analysis of PFS, provided the PFS shows a clinically and statistically significant improvement in PFS of the sotorasib and panitumumab arm(s) over the investigator's choice arm and the totality of the safety and efficacy data strongly favors the sotorasib and panitumumab arm(s). For subjects who previously stopped investigator's choice at progressive disease, as determined by BICR, they may be offered cross over to sotorasib or panitumumab if it is determined that crossover is appropriate after the primary analysis. Subjects who discontinue prior to BICR determined progressive disease will not be allowed to cross over unless they subsequently have BICR determined progressive disease before starting another systemic therapy. Subjects who withdraw consent will not be allowed to crossover. Subjects may discontinue treatment because of disease progression, intolerance of treatment leading to treatment discontinuation, initiation of another anticancer therapy or withdrawal of consent. Continued study treatment after radiological progression may be allowed if in the opinion of the investigator, the subject is deriving clinical benefit, is clinically stable, has no unacceptable toxicity from study drug, agrees to biopsy, and approval of the Medical Monitor is obtained. For subjects who continue treatment post-progression, tumor assessments will continue as per schedule until subject stops all study drugs.

For subjects who continue treatment post-progression or crossover, the date of first BICR assessed progression will be used for the primary PFS analysis and subject's tumor assessments post first progression will not be used for the primary analysis to evaluate objective response endpoints.

In all subjects, information regarding the type and duration of subsequent therapies following disease progression, response to subsequent therapy, date of progression on subsequent therapy, and survival data will be collected. Subjects who discontinue treatment prior to RECIST 1.1 disease progression (e.g., due to unacceptable toxicity) will continue to be followed radiographically until disease progression, withdrawal of consent, or start of another anti-cancer therapy, and then further long-term follow up via phone or visit to clinic for assessment of survival and documentation of anticancer treatment for subjects who have not withdrawn consent. Subjects will be followed for 1 year after last subject enrolled, or until withdrawal of consent, loss to follow-up, or subject death, whichever occurs first.

Interim safety analysis will be conducted by an independent data monitoring committee (DMC) after approximately 75 subjects have been enrolled and have had the opportunity to complete at least 8 weeks of study treatment, and then at approximately 6-month intervals until last subject is off treatment and then yearly until end of the study.

The assessments to be conducted in this study are described below and will be carried out at the timepoints designated in the schedule of assessment (SOA).

Disease progression will be assessed using Response Evaluation Criteria in Solid Tumors, Version 1.1 (RECIST v1.1) as assessed by BICR.

Safety will be monitored by assessing serious and non-serious adverse events (AEs), safety laboratory tests, vital signs and electrocardiogram (ECG). The incidence, nature and severity of all AEs will be graded according to the National Cancer Institute Common Terminology Criteria for Adverse Events, Version 5.0 (NCI CTCAE v5.0).

Laboratory safety tests will include hematology, blood chemistry, urinalysis, thyroid function, cholesterol, and triglycerides. Vital sign assessments will include blood pressure and pulse rate; additional vital signs will be collected only if clinically warranted.

PRO/QOL assessments will be assessed using the PRO instruments EORTC QLQ-C30, BPI, BFI, and questions from the PRO CTCAE, a single question about symptom bother (GP5 from the FACT G), and the EQ-5-D-5L. PRO scores will be determined for all patients for whom the PRO instruments are available in the patient's language at baseline and at timepoints designated in the SOA and will be compared across treatment groups. Patient Global Impression of Severity and Patient Global Impression of Change in Fatigue and Pain will be collected to facilitate interpretation of data from the BFI and BPI.

Samples will be collected for sotorasib and panitumumab PK analyses.

Samples will be collected for tumor markers.

Additional plasma/blood/tissue biopsy samples will be collected for exploratory biomarkers for mechanisms of primary and secondary resistance:
All subjects with lesions amenable to biopsy (core needle or fine needle aspiration (FNA)) at the time of progression will be encouraged to undergo biopsy at progression.
All subjects treated with sotorasib and panitumumab with lesions amenable to biopsy who wish to continue treatment past progression will be required to undergo a biopsy at progression if medically feasible prior to continuing treatment past progression.

An independent data monitoring committee (DMC) is planned for this study to review safety data as per DMC charter. Interim safety analysis will be conducted by DMC after approximately 75 subjects have been enrolled and have had the opportunity to complete at least 8 weeks of study treatment, and then at approximately 6-month intervals until last subject is off treatment and then approximately yearly until end of the study.

There will be one planned PFS primary analysis for superiority of PFS between the sotorasib and panitumumab arm and the investigator's choice arm. The PA of PFS will occur when approximately 60 PFS events have been observed from the sotorasib 960 mg and panitumumab arm and the investigator's choice arm.

### Study Products

Sotorasib 240 mg or 960 mg oral daily.

Panitumumab 6 mg/kg IV Q2W

Trifluridine and tipiracil 35 mg/m² up to a maximum of 80 mg per dose (based on the trifluridine component) oral twice daily within one hour of completion of morning and evening meals on Days 1-5 and 8-12 of each 28-day cycle. Round dose to the nearest 5 mg increment.

Regorafenib 160 mg orally daily×21 days of each 28-day cycle. Take after a low-fat meal.

### Inclusion Criteria:

Age ≥18 yrs.

Pathologically documented metastatic colorectal adenocarcinoma.

Have documentation of KRAS G12C mutation as determined by central testing.

Subjects will have received at least one prior line of therapy for metastatic disease. Subjects must have received and progressed or experienced disease recurrence on or after fluoropyrimidine, irinotecan, and oxaliplatin given for metastatic disease unless the subject, in the opinion of the investigator, is not a candidate for fluoropyrimidine, irinotecan, or oxaliplatin, in which case, the subject may be eligible after investigator discussion with medical monitor provided subject has received at least one prior line of therapy for metastatic disease. Subjects with tumors known to be MSI-H must have received prior checkpoint inhibitor therapy if available in the region unless there is a medical contraindication, in which case, the subject may be eligible after investigator discussion with medical monitor.

Subjects with tumors known to have BRAF V600E mutation must have received prior treatment with encorafenib and cetuximab if available for this indication in the country or region.
Adjuvant therapy will count as a line of therapy for metastatic disease if the subject progressed on or within six months of completion of adjuvant therapy administration.
Maintenance therapy is not considered as a separate regimen of therapy.
Adjuvant therapy given after resection of metastatic disease counts as a line of therapy for metastatic disease.
Perioperative chemotherapy with or without chemoradiation in the metastatic setting will count as one line of therapy for metastatic disease if that was part of a multidisciplinary treatment plan for surgery.

Subjects must be willing to provide archived tumor tissue samples (formalin-fixed paraffin-embedded [FFPE] sample collected within 5 years) or agree to undergo a pretreatment tumor biopsy (excisional or core biopsy) prior to enrollment.

Measurable disease per RECIST 1.1 criteria. Lesions previously radiated are not considered measurable unless they have progressed after radiation.

Eastern Cooperative Oncology Group (ECOG) Performance Status of ≤2.

Life expectancy of >3 months, in the opinion of the investigator.

Adequate hematologic and end-organ function, defined as the following within 2 weeks prior to cycle 1 day 1:
ANC ≥1.5×10⁹ cells/L (without granulocyte colony-stimulating factor support within 2 weeks of laboratory test used to determine eligibility).
Hemoglobin ≥9.0 g/dL (without transfusion within 2 weeks of laboratory test used to determine eligibility).
Platelet count ≥100×10⁹ cells/L (without transfusion within 2 weeks of laboratory test used to determine eligibility).
Aspartate aminotransferase (AST) and alanine transaminase (ALT) ≤2.5 times the upper limit of normal.
Serum bilirubin ≤1.0×ULN. For subjects with Gilbert's disease, direct bilirubin ≤1.0×ULN
International normalized ratio (INR) and activated partial thromboplastin time (or partial thromboplastin time) ≤1.5×ULN. Prothrombin time (PT) ≤1.5×ULN may be used instead of INR for sites whose labs do not report INR.
Estimated glomerular filtration rate based on Modification of Diet in Renal Disease (MDRD) calculation ≥30 ml/min/1.73 m².
Fridericia's Correction Formula (QTcF) ≥470 msec.

Ability to take oral medications and willing to record daily adherence to investigational product.

### Exclusion Criteria:

### Disease Related:

Active brain metastases. The phrase “active brain metastases” as used herein refers to a cancer that has spread from the original (primary, non-brain) tumor to the brain. Active brain metastases can be assessed by the presence of intracranial lesions. It is to be understood that while “metastases” is plural, patients exhibiting only one intracranial lesion under the criteria noted below is a patient who has “active brain metastases.” In some embodiments, a patient having active brain metastases has at least one measurable intracranial lesion >5 mm. In some embodiments, a patient having active brain metastases has at least one measurable intracranial lesion >5 mm but <10 mm. In some embodiments, a patient having active brain metastases has at least one measurable intracranial lesion >10 mm. A patient is not considered a patient with active brain metastases if the patient has had brain metastases resected or have received radiation therapy ending at least 4 weeks prior to study day 1 and are eligible if they meet all of the following criteria: a) residual neurological symptoms grade ≥2; b) on stable doses of dexamethasone or equivalent for at least 2 weeks, if applicable; and c) follow-up MRI performed within 28 days of Day 1 shows no progression or new lesions appearing. For determining the grade of any neurological symptom attributable to an intracranial lesion, see National Cancer Institute Common Terminology Criteria for Adverse Events v5.0 (NCI CTCAE) published Nov. 27, 2017 by the National Cancer Institute, incorporated herein by reference in its entirety.

### Other Medical Conditions:

History or presence of hematological malignancies unless curatively treated with no evidence of disease ≥2 years.

History of other malignancy within the past 3 years with the following exceptions:
Malignancy treated with curative intent and with no known active disease present for ≥3 years before enrollment and felt to be at low risk for recurrence by the treating physician.
Adequately treated non-melanoma skin cancer or lentigo maligna without evidence of disease.
Adequately treated cervical carcinoma in situ without evidence of disease.
Adequately treated breast ductal carcinoma in situ without evidence of disease.
Prostatic intraepithelial neoplasia without evidence of prostate cancer.
Adequately treated urothelial papillary non-invasive carcinoma or carcinoma in situ.

Leptomeningeal disease.

Significant GI disorder that results in significant malabsorption, requirement for IV alimentation, or inability to take oral medication.

History of interstitial pneumonitis or pulmonary fibrosis or evidence of interstitial pneumonitis or pulmonary fibrosis.

Significant cardiovascular disease, such as New York Heart Association cardiac disease (Class II or greater), myocardial infarction within 6 months prior to randomization, unstable arrhythmias or unstable angina

Significant uncontrolled concomitant disease that could affect compliance with protocol procedures or interpretation of results or that pose a risk to subject safety, in the opinion of the investigator or medical monitor.

Uncontrolled pleural effusion, pericardial effusion, or ascites requiring recurrent drainage procedures at a frequency greater than monthly. Subjects with PleurX catheters or intraperitoneal drainage catheters in place may be considered for the study with Medical Monitor approval.

Known history of human immunodeficiency virus (HIV) infection.

Exclusion of hepatitis infection based on the following results and/or criteria:
Positive hepatitis B surface antigen (HepBsAg) (indicative of chronic Hepatitis B or recent acute hepatitis B).
Negative HepBsAg with a positive for hepatitis B core antibody (Hepatitis B core antibody testing is not required for screening, however if this is done and is positive, then hepatitis B surface antibody [anti-HBs]testing is necessary. Undetectable anti HBs in this setting would suggest unclear and possible infection and needs exclusion).
Positive Hepatitis C virus antibody: Hepatitis C virus RNA by polymerase chain reaction is necessary. Detectable Hepatitis C virus RNA renders the subject ineligible.

If above antibody/antigen testing is not able to be obtained, positive hepatitis B or C viral load.

### Prior Concomitant Therapy:

Subject received trifluridine and tipiracil and regorafenib in the past.

Unresolved toxicities from prior anti-tumor therapy, defined as not having resolved to CTCAE version 5.0 grade 0 or 1, or to levels dictated in the eligibility criteria with the exceptions of alopecia (any grade allowed), neuropathy (up to grade 2 allowed), or toxicities from prior anti-tumor therapy that are considered irreversible [defined as having been present and stable for >6 months], or endocrine AEs that are stably maintained on appropriate replacement therapy.

Previous treatment with a KRAS^{G12C} inhibitor.

Prior treatment with trifluridine and tipiracil in those subjects where investigator's choice would be trifluridine and tipiracil.

Prior treatment with regorafenib in those subjects where investigator's choice would be regorafenib.

Therapeutic or palliative radiation therapy within 2 weeks of study day 1. Subjects must have recovered from all radiotherapy related toxicity to CTCAE version 5.0 grade 1 or less with the exception of alopecia (any grade of alopecia allowed).

Anti-tumor therapy (chemotherapy, antibody therapy, molecular targeted therapy, retinoid therapy, hormonal therapy [except for subjects with history of completely resected breast cancer with no active disease for over 3 years on long term adjuvant endocrine therapy], or investigational agent) within 4 weeks of study day 1; please note that bisphosphonates or anti-RANKL antibody therapy is allowed if needed for management of hypercalcemia or for prevention of skeletal events. Checkpoint inhibitor therapy within 6 weeks of study day 1 is also excluded.

Required dose reduction of panitumumab in the past for toxicity.

Use of warfarin. Other anticoagulation may be allowed.

Use of known cytochrome P450 (CYP) 3A4 sensitive substrates and P-glycoprotein (P-gp) substrates (with a narrow therapeutic window), within 14 days or 5 half-lives of the drug or its major active metabolite, whichever is longer, prior to study day 1 that was not reviewed and approved by the principal investigator and the medical monitor.

Use of strong inducers of CYP3A4 (including herbal supplements such as St. John's wort) within 14 days or 5 half-lives (whichever is longer) prior to study day 1 that was not reviewed and approved by the principal investigator and the medical monitor.

Where Investigator's choice is regorafenib: use of strong inhibitors of CYP3A4 (including herbal supplements such as Goldenseal) within 14 days or 5 half-lives (whichever is longer) or grapefruit juice or grapefruit containing products within 7 days prior to study day 1 that was not reviewed and approved by the principal investigator and the medical monitor.

Therapeutic oral or IV antibiotics within 2 weeks prior to randomization. Prophylactic antibiotics are allowed.

### Prior/Concurrent Clinical Study Experience:

Currently receiving treatment with another investigational device or drug study, or less than 4 weeks since ending another investigational device or investigational agent(s) or less than 6 weeks since receiving other investigational agent(s) on study if a checkpoint inhibitor was involved.

### Diagnostic Assessments:

Uncontrolled hypertension (systolic blood pressure >140 mm Hg or diastolic blood pressure >90 mm Hg) for subjects where investigator's choice is regorafenib.

### Other Exclusions:

Female subjects of childbearing potential unwilling to use protocol specified method of contraception during treatment and for an additional:
7 days after the last dose of sotorasib.
2 months after the last dose of panitumumab.
6 months after the last dose of trifluridine and tipiracil.
2 months after the last dose of regorafenib.

Female subjects who are breastfeeding or who plan to breastfeed while on study through:
7 days after the last dose of sotorasib.
2 months after the last dose of panitumumab.
6 months after the last dose of trifluridine and tipiracil.
2 months after the last dose of regorafenib.

Female subjects planning to become pregnant while on study through:
7 days after the last dose of sotorasib.
2 months after the last dose of panitumumab.
6 months after the last dose of trifluridine and tipiracil.
2 months after the last dose of regorafenib.

Female subjects of childbearing potential with a positive pregnancy test assessed at screening or day 1 by a highly sensitive urine or serum pregnancy test.

Male subjects with a female partner of childbearing potential who are unwilling to practice sexual abstinence (refrain from heterosexual intercourse) or use contraception during treatment and for an additional:
7 days after the last dose of sotorasib.
6 months after the last dose of trifluridine and tipiracil.
2 months after the last dose of regorafenib.

Male subjects with a pregnant partner who are unwilling to practice abstinence or use a condom during treatment and for an additional:
7 days after the last dose of sotorasib.
6 months after the last dose of trifluridine and tipiracil.
2 months after the last dose of regorafenib.

Male subjects unwilling to abstain from donating sperm during treatment and for an additional:
7 days after the last dose of sotorasib.
6 months after the last dose of trifluridine and tipiracil.
2 months after the last dose of regorafenib.

Major surgery within 28 days of study day 1.

Subject has known sensitivity to any of the products or components to be administered during dosing.

Subject likely to not be available to complete all protocol-required study visits or procedures, and/or to comply with all required study procedures to the best of the subject and investigator's knowledge.

History or evidence of any other clinically significant disorder, condition or disease (with the exception of those outlined above) that, in the opinion of the investigator or physician, if consulted, would pose a risk to subject safety or interfere with the study evaluation, procedures, or completion.

Excluded Treatments, Medical Devices, and/or Procedures During Study Period:

Anti-tumor therapy such as: (1) chemotherapy, antibody therapy, molecular targeted therapy, retinoid therapy, or hormonal therapy (except for subjects with breast cancer receiving it as adjuvant therapy); (2) therapeutic or palliative radiation therapy to non-target lesion(s) may be allowed for symptom control provided there is discussion and agreement between investigator and medical monitor prior to radiation therapy. Study drugs must be held during radiation therapy.

For those taking sotorasib or regorafenib: strong CYP3A4 inducers (including herbal supplements such as St. John's wort) unless approved by Medical Monitor.

For those taking regorafenib: strong CYP3A4 inhibitors (including grapefruit juice, grapefruit containing products, or herbal supplements such as Goldenseal) unless approved by Medical Monitor.

For those taking sotorasib: known CYP3A4 and/or P-gp sensitive substrates with narrow therapeutic index unless approved by Medical Monitor.

Other investigational agents

Anti-EGFR targeting agents other than panitumumab

### Objectives/Endpoints:

| | |
|---|---|
| Objectives | Endpoints |

Primary

| | |
|---|---|
| To compare progression free survival (PFS) in | PFS - defined as time from randomization until |
| previously treated subjects with KRAS G12C mutated | disease progression or death from any cause, |
| CRC receiving sotorasib 240 mg QD and | whichever occurs first, for all subjects. Progression |
| panitumumab vs investigator's choice (trifluridine and | unless noted otherwise will use RECIST version 1.1 |
| tipiracil or regorafenib), and sotorasib 960 mg QD | per blinded independent central review (BICR). |
| and panitumumab vs investigator's choice (trifluridine | |
| and tipiracil or regorafenib) | |

Secondary

| | |
|---|---|
| To compare overall survival (OS) in previously | Overall survival - defined as time from randomization |
| treated subjects with KRAS G12C mutated CRC | until death from any cause |
| receiving sotorasib 240 mg QD and panitumumab vs | |
| investigator's choice (trifluridine and tipiracil or | |
| regorafenib), and sotorasib 960 mg QD and | |
| panitumumab vs investigator's choice (trifluridine and | |
| tipiracil or regorafenib) | |
| To evaluate efficacy of sotorasib 240 mg QD and | Objective response = complete response [CR] + |
| panitumumab vs investigator's choice (trifluridine and | partial response [PR]), assessed per RECIST 1.1. |
| tipiracil or regorafenib), and sotorasib 960 mg QD | Response will be assessed by BICR. CR and PR |
| and panitumumab vs investigator's choice (trifluridine | require confirmatory repeat assessment at least 4 |
| and tipiracil or regorafenib), as assessed by: | weeks after the first detection of response. |
| Objective response rate (ORR) | |
| To evaluate efficacy of sotorasib 240 mg QD and | Duration of overall response- defined as time from |
| panitumumab vs investigator's choice (trifluridine and | first evidence of PR or CR to disease progression or |
| tipiracil or regorafenib), and sotorasib 960 mg QD | death due to any cause, whichever occurs first. |
| and panitumumab vs investigator's choice (trifluridine | Progression will be based on BICR or an |
| and tipiracil or regorafenib), as assessed by: | independent radiologic assessment of disease |
| Duration of response (DOR) | response per RECIST 1.1 |
| Time to response (TTR) | Time to response - defined as time from |
| Disease control rate (DCR) | randomization to first evidence of PR or CR based on |
| Investigator assessed ORR and PFS | BICR |
| | Disease control defined as CR + PR + stable disease |
| | of at least 7 weeks measured based on BICR |
| | PFS and ORR based on investigator assessment per |
| | RECIST 1.1 |
| To evaluate the safety and tolerability of sotorasib | Incidence and severity of treatment- emergent |
| 240 mg QD and panitumumab vs investigator's | adverse events, changes in vital signs, and changes |
| choice (trifluridine and tipiracil or regorafenib), and | in clinical laboratory tests |
| sotorasib 960 mg QD and panitumumab vs | |
| investigator's choice (trifluridine and tipiracil or | |
| regorafenib) | |
| To evaluate the impact of sotorasib 240 mg QD and | Change from baseline over time to |
| panitumumab, and sotorasib 960 mg QD and | week 8 |
| panitumumab vs investigator's choice (trifluridine and | Fatigue severity as measured by Item 3 of the |
| tipiracil or regorafenib) on patient-reported fatigue, | Brief Fatigue Inventory (BFI) |
| pain, physical function, and global health status | Pain severity as measured by item 3 of the |
| (patient reported outcomes [PRO]) | Brief Pain Inventory (BPI) |
| | Physical functioning as measured by the |
| | physical function domain of the European |
| | Organisation for Research and Treatment of Cancer |
| | Core Quality of Life Questionnaire Core |
| | Questionnaire (EORTC QLQ C30) |
| | Global health status as measured by |
| | Questions 29 and 30 of the EORTC QLQ C30 |
| To evaluate and compare the effect of treatment with | Change from baseline over time to 8 weeks for all |
| sotorasib 240 mg and panitumumab vs investigator's | subscales of the Brief Fatigue and Pain Inventory |
| choice (trifluridine and tipiracil or regorafenib), and | Subscales and remaining of the subscales of EORTC |
| sotorasib 960 mg QD and panitumumab vs | QLQ-C30 |
| investigator's choice (trifluridine and tipiracil or | Summary scores at each assessment and changes |
| regorafenib) on other treatment and disease related | from baseline of visual analog scales (VAS) scores |
| symptoms and health related quality of life relative to | as measured by EuroQol-5D level 5 EQ5D-5L |
| investigator's choice (trifluridine and tipiracil or | Summary scores on single question on symptom |
| regorafenib) | bother 5-item Guilt Proneness Scale (GP5) from |
| | Functional Assessment of Cancer Therapy - General |
| | (FACT G) |
| | Summary scores of Patient Global Impression of |
| | change (1 question each for fatigue and pain) at each |
| | protocol specified timepoint |
| To characterize the pharmacokinetics (PK) of | Pharmacokinetic (PK) parameters of sotorasib and |
| sotorasib and panitumumab | panitumumab including, but not limited to, maximum |
| | plasma concentration (Cₘₐₓ), area under the plasma |
| | concentration-time curve (AUC) |

Exploratory objectives

| | |
|---|---|
| To evaluate the association of biomarker levels and | Protein, ribonucleic acid (RNA), and/or |
| clinical activity | deoxyribonucleic acid (DNA) biomarkers in blood and |
| | tumor samples. |
| | Clinical response including but not limited to OS, |
| | PFS, DOR, objective response |
| To evaluate the time to deterioration in fatigue and | Time to deterioration in severity of |
| pain between treatment with sotorasib 240 mg QD | pain and fatigue for the Brief Fatigue |
| and panitumumab relative to investigator's | and Pain Inventory Subscales and all |
| choice (trifluridine and tipiracil or regorafenib), and | subscales in the EORTC QLQ-C30 |
| sotorasib 960 mg QD and panitumumab relative to | |
| investigator's choice (trifluridine and tipiracil or | |
| regorafenib) | |

### Sotorasib Dosage Adjustments, Delays, Rules for Withholding or Restarting, Permanent Discontinuation

The sotorasib starting dose to be used in the study will be 960 or 240 mg/day. Sotorasib will be administered orally QD for a treatment cycle of 28 days with or without food. Subject should take the sotorasib dose (all tablets at the same time) with or without food at approximately the same time every day. The sotorasib dose should also not be taken more than 2 hours earlier than the target time based on previous day's dose. If 6 hours have passed from the scheduled time of dosing, the dose should be skipped for that day. Take the next dose as prescribed the next day. Do not take 2 doses at the same time to make up for the missed dose. If vomiting occurs after taking sotorasib, do not take an additional dose. Take the next dose as prescribed the next day.

Administration to Subjects Who Have Difficulty Swallowing Solids: Disperse tablets in 120 mL (4 ounces) of non-carbonated, room-temperature water without crushing. No other liquids should be used. Stir until tablets are dispersed into small pieces (the tablets will not completely dissolve) and drink immediately or within 2 hours. The appearance of the mixture may range from pale yellow to bright yellow. Swallow the tablet dispersion. Do not chew pieces of the tablet. Rinse the container with an additional 120 mL (4 ounces) of water and drink. If the mixture is not consumed immediately, stir the mixture again to ensure that tablets are dispersed.

Dose modification sotorasib for toxicity management of individual subjects is provided in the following table.

Sotorasib Doses (mg QD)

| Starting Dose | Dose -1 | Dose -2 |
|---|---|---|
| 960 | 480 | 240 |

QD = once daily

Subjects receiving 240 mg of sotorasib will be allowed up to 2 dose interruptions, but sotorasib will not be dose reduced upon resuming sotorasib if deemed medically safe and appropriate per the investigator's opinion. Subjects in the 960 mg sotorasib treatment arm who require more than 2 dose reductions due to toxicity management related to sotorasib or are in the 240 mg sotorasib treatment arm who require more than 2 dose interruptions due to toxicity management related to sotorasib should be permanently discontinued from sotorasib treatment.

Sotorasib Dose Modification Guidelines for Sotorasib-related Hematologic and
Non-Hematologic Toxicities

| | Recommended Action | |
|---|---|---|
| Toxicity | Hold Until: | Restart Dose: |
| Grade ≥3 nausea, vomiting, or | Recovery to grade 1 or less or | resume dosing at 1 dose |
| diarrhea lasting longer than | to baseline grade | lower^{a} |
| 3 days despite optimal medical | | |
| support | | |
| Any other drug-related toxicity | Recovery to grade 1 or less or | resume dosing at 1 dose |
| ≥grade 3^{b} | to baseline grade | lower^{a} |

^{a}Subjects may be resumed at a dose lower than the recommended restarting dose after discussion with the Medical Monitor
^{b}For subjects with hepatotoxicity, see below

If sotorasib is held, panitumumab should be held as well.

The following stopping and/or withholding rules apply to subjects for whom another cause of their changes in liver biomarkers (TBL, INR and transaminases) has not been identified. Important alternative causes for elevated AST/ALT and/or TBL values include, but are not limited to: Hepatobiliary tract disease; Viral hepatitis (e.g., hepatitis A/B/C/DIE, Epstein-Barr Virus, cytomegalovirus, herpes simplex virus, varicella, toxoplasmosis, and parvovirus); Right sided heart failure, hypotension or any cause of hypoxia to the liver causing ischemia; Exposure to hepatotoxic agents/drugs or hepatotoxins, including herbal and dietary supplements, plants and mushrooms; Heritable disorders causing impaired glucuronidation (e.g., Gilbert's syndrome, Crigler-Najjar syndrome) and drugs that inhibit bilirubin glucuronidation (e.g., indinavir, atazanavir); Alpha-one antitrypsin deficiency; Alcoholic hepatitis; Autoimmune hepatitis; Wilson's disease and hemochromatosis; Nonalcoholic fatty liver disease including steatohepatitis; and/or Non-hepatic causes (e.g., rhabdomyolysis, hemolysis).

Rechallenge may be considered if an alternative cause for impaired liver tests (ALT, AST, ALP) and/or elevated TBL, is discovered and/or the laboratory abnormalities resolve to normal or baseline, as described in the below.

| Analyte | Temporary Withholding | Permanent Discontinuation |
|---|---|---|
| TBL | >3× ULN | >2× ULN |
| | at any time | OR |
| INR | — | >1.5× (for subjects not on anticoagulation therapy) |
| | OR | AND |
| AST/ALT | >5× ULN at any time | In the presence of no important alternative |
| | >3× ULN with clinical signs or symptoms that are | causes for elevated AST/ALT and/or TBL |
| | consistent with hepatitis (such as right upper | values |
| | quadrant pain/tenderness, fever, nausea, | >3× ULN (when baseline was <ULN) |
| | vomiting, and jaundice) | |
| | OR | |
| ALP | >8× ULN at any time | — |

ALP = alkaline phosphatase; ALT = alanine aminotransferase; AST = aspartate aminotransferase; INR = international normalized ratio; TBL = total bilirubin; ULN = upper limit of normal

Hepatotoxicity Guidelines for Sotorasib: Guidelines for management and monitoring of subjects with increased AST, ALT, or alkaline phosphatase (ALP) are presented in the table below.

If the conditions for permanent discontinuation are met (below): Participant to be permanently discontinued
AST or ALT >3× ULN and INR > 1.5× ULN (for subjects not on anticoagulation therapy) in the presence of
no important alternative causes for elevated AST/ALT values
OR
AST or ALT > 3× ULN and TBL > 2× ULN in the presence of no important alternative causes for elevated
AST/ALT and/or TBL values
If conditions are not met: Exclude other causes^{a}
Upon failure to identify any other causes and sotorasib relation to increase in LFTs cannot be excluded,
proceed with guidelines below:

| | Sotorasib | Medical | |
|---|---|---|---|
| CTCAE Grade | Action | Management | Monitoring and Follow-up |
| Grade 2 AST or ALT and | Continue | Consider steroids^{b} | Closely monitor liver function tests |
| ALP ≤ 8× ULN with | | | |
| no clinical symptoms | | | |
| consistent with hepatitis | | | |
| (right upper quadrant | | | |
| pain/tenderness, fever, nausea, | | | |
| vomiting, and jaundice) | | | |
| Grade 2 AST or ALT | First Occurrence | Initiate steroids^{b} | Closely monitor liver function tests |
| with symptoms | Withhold | | Await resolution to baseline or grade 1 |
| Or | | | and resolution or improvement of |
| Grade 3 or 4 | | | hepatitis symptoms |
| AST or ALT | | | Restart at 1 dose level reduction^{c,e} |
| Or | Second | Initiate steroids^{b} | Closely monitor liver function tests |
| 8× ULN ALP^{d} | Occurrence | | Await resolution to baseline or grade 1 |
| | Withhold | | and resolution or improvement of |
| | | | hepatitis symptoms |
| | | | Resume at an additional 1 dose level |
| | | | reduction only with MEDICAL MONITOR |
| | | | approval^{c,e} |

| | |
|---|---|
| Third Occurrence | NOT APPLICABLE |
| Permanently | |
| discontinue | |
| Sotorasib | |

ALP = alkaline phosphatase; ALT = alanine aminotransferase; AST = aspartate aminotransferase; CTCAE = Common Terminology Criteria for Adverse Events; INR = international normalized ratio; LFT = liver function test; TBL = total bilirubin; ULN = upper limit of normal
^{a}If increase in AST/ALT is likely related to alternative agent, discontinue causative agent and await resolution to baseline or grade 1 prior to resuming sotorasib.
^{b}For example: prednisone 0.25 to 1.0 mg/kg/day or equivalent, followed by a taper.
^{c}Close monitoring at restart (e.g., daily LFTs × 2, then weekly × 4). Sotorasib dose may be increased after discussion with Medical Monitor.
dThere is no limit to the number of sotorasib re-challenges for isolated alkaline phosphatase elevations that resolve to baseline or grade 1.
^{e}Dose decrements below 240 mg are not allowable. Subjects may restart at same dose without dose reduction.

Hepatotoxicity Response: Subjects with abnormal hepatic laboratory values (i.e., alkaline phosphatase (ALP), aspartate aminotransferase (AST), alanine aminotransferase (ALT), total bilirubin (TBL)) and/or international normalized ratio (INR) and/or signs/symptoms of hepatitis (as described below) may meet the criteria for withholding or permanent discontinuation of sotorasib.

### Panitumumab Dosage Adjustments, Delays, Rules for Withholding or Restarting, Permanent Discontinuation

The starting panitumumab dose to be used in the study will be 6 mg/kg Q2W. Panitumumab will be administered as an IV infusion over 60-minutes (≤1000 mg) or 90-minutes (>1000 mg) Q2W. If the first infusion is tolerated, administer subsequent infusions over 30 to 60 minutes. To be administered using a low-protein binding 0.2 or 0.22 Om in-line filter.

Panitumumab should be administered 2 hours (+30 minutes) after sotorasib on Cycle 1 Day 1. For subsequent doses of panitumumab, there is no need to wait 2 hours. Panitumumab can be administered immediately after sotorasib starting C1D15. After cycle 1, there is no requirement for sotorasib to be administered prior to panitumumab as long as it is given that day in the allowed window. Panitumumab dose is calculated based on weight on day 1 of each cycle. However, if it is institutional policy, panitumumab dose recalculation is not required if the subject's weight changes by <10%.

For subjects who experience toxicities while on study, 1 or more doses of panitumumab are withheld, reduced, or delayed (administered at >14 day intervals). Exemplary panitumumab dose reductions are listed in the table below.

| | | First Dose | Second Dose |
|---|---|---|---|
| | Starting Dose | Reduction | Reduction |
| Percentage (%) | 100 | 80 | 60 |
| mg/kg | 6 | 4.8 | 3.6 |

If panitumumab is held, sotorasib may continue if determined to be clinically safe by the Investigator.

Exemplary panitumumab dose modification guidelines due to dermatological toxicities are provided in the table below.

| Occurrence of Skin | | | |
|---|---|---|---|
| Symptom(s): | Administration of | | |
| ≥grade 3 | Panitumumab | Outcome | Dose Regulation |
| Initial occurrence | Stop 1 or 2 doses | Improved (<grade 3) | Continue infusion at 100% |
| | | | of original dose |
| | | Not recovered | Discontinue |
| At the second | Stop 1 or 2 doses | Improved (<grade 3) | Continue infusion at 80% of |
| occurrence | | | original dose |
| | | Not recovered | Discontinue |
| At the third occurrence | Stop 1 or 2 doses | Improved (<grade 3) | Continue infusion at 60% of |
| | | | original dose |
| | | Not recovered | Discontinue |
| At the fourth | Discontinue | — | — |
| occurrence | | | |

In the event of severe or life-threatening inflammatory or infectious complications, consider withholding or discontinuing panitumumab as clinically appropriate.

Subjects starting panitumumab should be started on skin prophylaxis prior to the first dose of panitumumab if feasible. Skin prophylaxis, as clinically indicated, should include skin moisturizer, sunscreen (sun protection factor [SPF]>15 ultraviolet A [UVA] and ultraviolet B [UVB]), topical steroid cream (not stronger than 1% hydrocortisone) and an oral antibiotic (doxycycline 100 mg BID or minocycline 100 mg QD) (Lacouture et al, 2010). Details of frequency of application and sites of application of these prophylactic measures can be found in the prescribing information and information brochure for panitumumab.

Treatment of skin reactions should be based on severity and may include a moisturizer, sunscreen (SPF >15 UVA and UVB), and topical steroid cream (not stronger than 1% hydrocortisone) applied to affected areas, and/or oral antibiotics, as prescribed by the physician. If the skin reaction does not resolve with these measures, additional measures may be used per institutional guidelines for management of dermatological toxicities.

### Pulmonary Toxicity

In the event of acute onset or worsening of pulmonary symptoms, consider withholding panitumumab. If interstitial lung disease is confirmed, discontinue panitumumab.

### Eye Toxicity

Patients who develop eye toxicities while receiving panitumumab should be monitored for evidence of keratitis or ulcerative keratitis. If a diagnosis of ulcerative keratitis is confirmed, treatment with panitumumab should be interrupted or discontinued. If keratitis is diagnosed, the benefits and risks of continuing treatment should be carefully considered. Subjects presenting with signs and symptoms suggestive of keratitis such as acute or worsening: eye inflammation, lacrimation, light sensitivity, blurred vision, eye pain and/or red eye should be referred promptly to an ophthalmology specialist.

### Hypersensitivity Reactions

Depending on the severity (e.g., presence of bronchospasm, edema, angioedema, hypotension, need for parenteral medication, or anaphylaxis) and/or persistence, of hypersensitivity reactions, permanently discontinue panitumumab.

Toxicities Other than Dermatologic or Pulmonary

Withhold panitumumab for any grade 3 or 4 panitumumab-related toxicity with the following exceptions:
Panitumumab will only be withheld for symptomatic grade 3 or 4 hypomagnesemia and/or hypocalcemia that persists despite aggressive magnesium and/or calcium replacement
Panitumumab will only be withheld for grade 3 or 4 nausea, diarrhea, or vomiting that persists despite maximum supportive care

### Infusion Reactions

Infusion reaction may manifest as fever, chills, dyspnea, bronchospasm, or hypotension.
Reduce infusion rate by 50% in patients experiencing a mild or moderate (grade 1 or 2) infusion reaction for the duration of that infusion. Subsequent infusions may be administered at the reduced infusion rate if needed.
Terminate the infusion in patients experiencing severe infusion reactions. Depending on the severity and/or persistence of the reaction, permanently discontinue panitumumab.
   Criteria for Re-Treatment with Panitumumab

For toxicities other than dermatologic: If panitumumab was withheld, administration may recommence once the adverse event has improved to grade 1 or returned to baseline.

### Trifluridine and Tipiracil

The dose of trifluridine and tipiracil is calculated on cycle 1 day 1 based on the body surface area (BSA). At the start of subsequent cycles, if the weight changes by more than or equal to 10%, the BSA should be recalculated and used for dosing.

Obtain complete blood cell counts prior to and on day 15 of each cycle.

Do not initiate the cycle of trifluridine and tipiracil until:
ANC is greater than or equal to 1 500/mm³ or febrile neutropenia is resolved
Platelets are greater than or equal to 75 000/mm³
Grade 3 or 4 non-hematological adverse reactions are resolved to Grade 0 or 1

Within a treatment cycle, withhold trifluridine and tipiracil for any of the following:
ANC less than 500/mm³ or febrile neutropenia
Platelets less than 50 000/mm³
Grade 3 or 4 non-hematological adverse reactions

After recovery, resume trifluridine and tipiracil after reducing the dose by 5 mg/m2/dose from the previous dose level, if the following occur:
Febrile neutropenia
Uncomplicated grade 4 neutropenia (which has recovered to greater than or equal to 1,500/mm³) or thrombocytopenia (which has recovered to greater than or equal to 75,000/mm³) that results in more than 1 week delay in start of next cycle
Non-hematologic grade 3 or grade 4 adverse reaction except for grade 3 nausea and/or vomiting controlled by antiemetic therapy or grade 3 diarrhea responsive to antidiarrheal medication

If in the opinion of the investigator, a more severe dose reduction or dose hold is indicated, that is permissible.

A maximum of 3 dose reductions are permitted to a minimum dose of 20 mg/m2 twice daily. Do not escalate trifluridine and tipiracil dose after it has been reduced.

### Regorafenib

Severe and sometimes fatal hepatotoxicity has occurred in clinical trials (see Regorafenib USPI). Monitor hepatic function prior to and during treatment. Interrupt and then reduce or discontinue regorafenib for hepatotoxicity as manifested by elevated liver function tests or hepatocellular necrosis, depending upon severity and persistence.

If dose modifications are required, reduce the dose in 40 mg (1 tablet) increments. If in the opinion of the investigator, a more severe dose reduction or dose hold is indicated, that is permissible. The lowest recommended daily dose of regorafenib is 80 mg daily.

Interrupt regorafenib for the following:
Grade 2 hand-foot skin reaction (HFSR) [palmar-plantar erythrodysesthesia syndrome (PPES)] that is recurrent or does not improve within 7 days despite dose reduction; interrupt therapy for a minimum of 7 days for grade 3 HFSR
Symptomatic grade 2 hypertension
Any grade 3 or 4 adverse reaction
Worsening infection of any grade

Reduce the dose of regorafenib to 120 mg:
For the first occurrence of grade 2 HFSR of any duration
After recovery of any grade 3 or 4 adverse reaction except infection
For grade 3 AST/ALT elevation, only resume if the potential benefit outweighs the risk of hepatotoxicity

Reduce the dose of regorafenib to 80 mg:
For re-occurrence of grade 2 HFSR at the 120 mg dose
After recovery of any grade 3 or 4 adverse reaction at the 120 mg dose (except hepatotoxicity or infection)

Discontinue regorafenib permanently for the following:
Failure to tolerate 80 mg dose
Any occurrence of AST or ALT more than 20 times the ULN
Any occurrence of AST or ALT more than 3 times ULN with concurrent bilirubin more than 2 times ULN
Re-occurrence of AST or ALT more than 5 times ULN despite dose reduction to 120 mg
For any grade 4 adverse reaction; only resume if the potential benefit outweighs the risks

### Efficacy Assessments

The extent of disease will be evaluated by contrast-enhanced CT/MRI according to RECIST 1.1 (discussed below). All radiological imaging will be performed as indicated in the Site Imaging Manual provided by the central imaging core laboratory. In order to reduce radiation exposure for subjects, low dose CT should be utilized whenever possible.

The screening scans must be performed within 28 days prior to cycle 1 day 1. If there are multiple screening scans, the one(s) closest to cycle 1 day 1 will be used as baseline. Imaging performed as part of standard of care prior to signing of informed consent may be used provided it was performed within 28 days of study start and are available for submission to the central imaging core laboratory.

Radiological assessment must include CT/MRI of the chest, abdomen and pelvis, as well as assessment of all other known sites of disease as detailed within the Site Imaging Manual.

All subjects with history of brain metastasis must have MRI of the brain performed. All brain scans for subjects with brain metastasis are required to be MRI unless MRI is contraindicated, and then CT with contrast is acceptable. Brain imaging (MRI or CT) should be performed if signs or symptoms suggestive of central nervous system metastases are present.

All subsequent scans should be performed in the same manner (e.g., with the same contrast, MRI field strength) as at screening preferably on the same scanner. If the imaging modality must be altered (e.g., unscheduled assessment) consultation with the Amgen medical monitor is recommended.

During treatment and follow-up, radiological imaging of the chest, abdomen, pelvis, as well as all other known sites of disease, will be performed independent of treatment cycle. Imaging may also be performed more frequently if clinically necessitated at the discretion of the managing physician. Confirmed radiographic response (complete response, PR) requires confirmation by a repeat scan at least 4 weeks after the first documentation of response, but may be performed later at the next scheduled scan. Radiologic imaging and tumor assessment will be performed until start of another anticancer therapy, withdrawal of consent, loss to follow-up, or death, whichever occurs earliest.

Scans will be submitted to a central imaging core laboratory for archival, response assessment including RECIST 1.1, and/or exploratory analysis (e.g., volumetric and viable tumor measurements). BICR continues to perform tumor assessments on obtained scans until the first BICR assessed progression is achieved. Scans obtained after first BICR assessed progression will not be routinely assessed by BICR.

Response Evaluation Criteria in Solid Tumors Version 1.1 (RECIST 1.1)

### Definitions:

### Measurable Lesions

Measurable Non-nodal Tumor Lesions

Non-nodal lesions with clear borders that can be accurately measured in at least 1 dimension with longest diameter ≥10 mm in computed tomography (CT)/MRI scan with slice thickness no greater than 5 mm. When slice thickness is greater than 5 mm, the minimum size of measurable lesion should be twice the slice thickness.

### Nodal Lesions

Lymph nodes are to be considered measurable if >15 mm in short axis when assessed by CT/MRI (scan slice thickness recommended to be no greater than 5 mm). At baseline and in follow-up, only the short axis will be measured and followed.

### Cystic Lesions

Cystic lesions thought to represent cystic metastases can be considered as measurable lesions, if they meet the definition of measurability described above for non-nodal lesions.

Bone Lesions with Identifiable Soft Tissue Components

Bone lesions with identifiable soft tissue components, that can be evaluated by cross sectional imaging techniques such as CT or MRI can be considered as measurable lesions if the soft tissue component meets the definition of measurability described above for non-nodal lesions.

### Clinically Measured Lesions

Visible or palpable lesions can be considered measurable if 10 mm in longest diameter for non-nodal or 15 mm in shortest diameter for lymph nodes. Lesions should be measured radiologically if more accurate, if not then measured by calipers.

### Irradiated Lesions

Tumor lesions situated in a previously irradiated area, or in an area subjected to other loco-regional therapy, are not measurable unless there has been demonstrated progression that is measurable in the lesion prior to enrollment.

### Non-Measurable Lesions

All other lesions, including small lesions (longest diameter <10 mm or pathological lymph nodes with ≥10 mm to <15 mm short axis with CT scan slice thickness no greater than 5 mm) are considered non measurable. (When slice thickness is greater than 5 mm, the minimum size of measurable lesion should be twice the slice thickness)

Other examples of lesions usually considered to be non-measurable include:
Lesions with prior local treatment: tumor lesions situated in a previously irradiated area, or an area subject to other loco-regional therapy, should not be considered measurable unless there has been demonstrated progression in the lesion.
Categorically, clusters of small lesions, bone lesions without a soft tissue component, inflammatory breast disease, ascites, pleural/pericardial effusions, lymphangitis cutis/pulmonitis, and leptomeningeal disease are non-measurable.

### Methods of Measurement

CT/MRI—Contrast-enhanced CT or MRI should be used to assess all lesions. Optimal visualization and measurement of metastasis in solid tumors requires consistent administration (dose and rate) of IV contrast as well as timing of scanning. CT and MRI should be performed with ≤5 mm thick contiguous slices.

PET-CT—At present, the low dose or attenuation correction CT portion of a combined positron emission tomography (PET)-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time

### Baseline Documentation of “Target” and “Non-Target” Lesions

Target Lesions—All measurable lesions up to a maximum of two (2) lesions per organ and five (5) lesions in total, representative of all involved organs should be identified as target lesions and recorded and measured at baseline.

Target lesions should be selected on the basis of their size (lesions with the longest diameter) and suitability for accurate repeated measurements.

Pathologic lymph nodes (with short axis ≥15 mm) may be identified as target lesions. All other pathological nodes (those with short axis ≥10 mm but <15 mm) should be considered non-target lesions.

Lymph nodes are considered one organ, thus a maximum of 2 measurable lymph nodes may be identified as target lesions.

A sum of the diameters (longest for non-nodal lesions, short axis for nodal lesions) for all target lesions will be calculated and reported as the baseline sum of diameters. The baseline sum of diameters will be used as reference by which to characterize objective tumor response.

Non-Target Lesions—All other lesions (or sites of disease) including pathological lymph nodes should be identified as non-target lesions and should also be recorded at baseline. Measurements of these lesions are not required, and these lesions should be followed as “present”, “absent”, or “unequivocal progression” throughout the study. In addition, it is possible to record multiple non-target lesions involving the same organ as a single item on the case report form (e.g., “multiple enlarged pelvic lymph nodes” or “multiple liver metastases”).

### Response Criteria

### Evaluation of Target Lesions

| | |
|---|---|
| *Complete Response (CR): | Disappearance of all target non-nodal lesions. Any target lymph nodes |
| | must have reduction in short axis to <10 mm, not total |
| | disappearance. |
| *Partial Response (PR): | At least a 30% decrease in the sum of the diameters of target lesions, |
| | taking as reference the baseline sum of diameters. |
| *Progressive Disease (PD): | At least a 20% increase in the sum of the diameters of target lesions, |
| | taking as reference the smallest sum on study (this includes the |
| | baseline sum if that is the smallest on study). In addition to the |
| | relative increase of 20%, the sum must also demonstrate an |
| | absolute increase of at least 5 mm. If a subject is missing lesion |
| | data at a disease assessment and yet PD criteria is met despite the |
| | missing data, the subject will be classified as PD . . . |
| *Stable Disease (SD) | Neither sufficient shrinkage to qualify for PR nor sufficient increase to |
| | qualify for PD |
| Not Evaluable (NE) | When inadequate or no imaging/measurement is done at a particular |
| | time point, the subject's response is not evaluable (NE) at that time |
| | point. |

### Evaluation of Non-Target Lesions

| | |
|---|---|
| *Complete Response (CR): | Disappearance of all non-nodal target lesions and normalization of |
| | tumor marker levels. All lymph nodes must be non-pathological in |
| | size (<10 mm short axis). |
| Non-CR/Bon-PD | Persistence of one or more non-target lesion(s) or/and maintenance |
| | of tumor marker levels above the normal limits. |
| *Progressive Disease (PD) | Unequivocal progression of existing non-target lesions and/or |
| | appearance of one or more new lesions.1 If a subject is missing |
| | lesion data at a disease assessment and yet unequivocal |
| | progression is met despite the missing data, the subject will be |
| | classified as PD. |
| Not Evaluable (NE) | When inadequate or no imaging is done at a particular time point, the |
| | subject's response is not evaluable (NE) at that time point. |

1To achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of 1 or more non-target lesions is usually not sufficient to qualify for unequivocal status.

### Evaluation of Best Overall Response

The subject's best response assignment will depend on the findings of both target and non target disease and will also take into consideration the appearance of new lesions and confirmation of response. Best overall response (BOR) will be the best response recorded based on all post baseline disease assessments that occur prior to disease progression and the initiation of subsequent anticancer treatment. At least 7 weeks from the first dose of study drug tumor assessment must elapse without radiological disease progression to meet the minimum criteria for stable disease (SD) duration in order to assign a best overall response of SD. In general, subjects not classifiable under the RECIST 1.1 response categories due to inadequate data or early death will be classified as non evaluable (NE) for BOR but will be counted in the denominator of all response rate calculations.

| | | | |
|---|---|---|---|
| Target Lesions | Non-target Lesions | New Lesions | Overall Response |

Subjects with Target (±Non-target) Disease

| | | | |
|---|---|---|---|
| CR | CR or NA | No | CR |
| CR | Non-CR/non-PD or NE | No | PR |
| CR | NE | No | PR |
| PR | CR or Non-CR/Non-PD | No | PR |
| | or NE or NA | | |
| SD | CR or Non-CR/Non-PD | No | SD |
| | or NE or NA | | |
| PD | Any | Any | PD |
| Any | PD | Any | PD |
| Any | Any | Yes | PD |
| NE | CR or Non-CR/Non-PD | No | NE |
| | or NE or NA | | |

Subjects with Non-target Disease Only

| | | | |
|---|---|---|---|
| NA | CR | No | CR |
| NA | Non-CR/Non-PD | No | Non-CR/Non-PD^{a} |
| NA | CR or Non-CR/Non-PD | NE | Non-CR/Non-PD^{a} |
| NA | PD | Any | PD |
| NA | Any | Yes | PD |
| NA | NE | No | NE |

CR = complete response; PD = progressive disease; PR = partial response; NE = Not evaluable; SD = stable disease.
^{a}Per RECIST 1.1, “Non-CR/Non-PD” is preferred over “SD” for Non-Target disease since SD is increasingly used as endpoint for assessment of efficacy in some trials so to assign this category when no lesions can be measured is not advised.

Best Overall Response When Confirmation of Complete Response (CR)
and Partial Response (PR) Required^{a}

| Overall Response | Overall Response | |
|---|---|---|
| First Time Point | Second Time Point | Best Overall Response |
| CR | CR | CR |
| CR | PR | SD, PD or PR^{b} |
| CR | SD | SD provided minimum criteria for SD duration |
| | | met, otherwise, PD |
| CR | PD | SD provided minimum criteria for SD duration |
| | | met, otherwise, PD |
| CR | NE | SD provided minimum criteria for SD duration |
| | | met, otherwise, NE |
| PR | CR | PR |
| PR | PR | PR |
| PR | SD | SD |
| PR | PD | SD provided minimum criteria for SD duration |
| | | met, otherwise, PD |
| PR | NE | SD provided minimum criteria for SD duration |
| | | met, otherwise, NE |
| NE | NE | NE |

CR = complete response; PD = progressive disease; PR = partial response; NE = Not evaluable; SD = stable disease.
^{a}If a CR is truly met at first time point, then any disease at a subsequent time point (see Confirmation section below for timing), even disease meeting PR criteria relative to baseline, makes the disease PD at that point (since disease must have reappeared after CR). Best response would depend on whether minimum duration for SD was met. However, sometimes “CR” may be claimed when subsequent scans suggest small lesions were likely still present and in fact that subject had PR, not CR at the first time point. Under these circumstances, the original CR should be changed to PR and the best response is PR.
^{b}Confirmed radiographic response (complete response, PR) requires confirmation by a repeat scan at least 4 weeks after the first documentation of response, but may be performed later at the next scheduled scan. Radiologic imaging and tumor assessment will be performed until BICR assessed progressive disease, start of another anticancer therapy, withdrawal of consent, loss to follow-up, or death, whichever occurs earliest.

### Special Notes on Response Assessment

Target lesions that become “too small to measure”—While on study, all lesions (nodal and non-nodal) recorded at baseline should have their measurements recorded at each subsequent evaluation, even when very small (e.g., 2 mm). However, sometimes lesions or lymph nodes which are recorded as target lesions at baseline become so faint on CT scan that the radiologist may not feel comfortable assigning an exact measure and may report them as being ‘too small to measure’. When this occurs, it is important that a value be recorded on the case report form. If it is the opinion of the radiologist that the non-lymph node lesion has likely disappeared, the measurement should be recorded as 0 mm. If the lesion is believed to be present and is faintly seen but too small to measure, a default value of 5 mm should be assigned (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well). This default value is derived from the 5 mm CT slice thickness (but should not be changed with varying CT slice thickness). The measurement of these lesions is potentially non reproducible, therefore providing this default value will prevent false responses or progressions based upon measurement error. To reiterate, however, if the radiologist is able to provide an accurate measure, that should be recorded, even if it is below 5 mm.

New lesions—The term “new lesion” always refers to the presence of a new finding that is definitely tumor. If a new lesion is identified via a modality other than CT or MRI, CT or MRI confirmation is recommended unless the new lesion is deemed unequivocally tumor. New findings that are not definitively tumor but may be benign (infection, inflammation, etc.) are not selected as new lesions, until that time when the review is certain they represent tumor.
If a new lesion is equivocal, for example because of its small size, continued therapy, and follow-up evaluation will clarify if it represents truly new disease. If additional imaging confirms there is definitely a new lesion, then progression should be declared using the date of the initial scan.
A lesion identified on a follow-up study in an anatomical location that was not scanned at baseline is considered a new lesion and will indicate disease progression, regardless of any response that may be seen in target or non-target lesions present from baseline.

Any locoregional therapy not allowed per protocol:
Any subject receiving locoregional therapy not allowed in the protocol while on study that directly affects one or more of the target lesions selected at baseline will be considered to be non-evaluable at all disease assessments that occur on or after the date of locoregional therapy with the exception of disease progression. However, if a lesion was completely resected where pathology was benign the subject will still be evaluable for response with 0 dimension reported.
If locoregional therapy was performed on a non-target lesion, that lesion will always be assessed as present unless pathology was benign.

Lesions that split or coalesce on treatment—When non-nodal lesions “fragment”, the longest diameters of the fragmented portions should be added together to calculate the target lesion sum and identified as a fragment of the original lesion. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the “coalesced lesion”.
“Symptomatic deterioration” alone does not qualify as objective progression. If objective progression was not previously documented, then every effort should be made to document objective progression even after discontinuation of treatment.
In some circumstances it may be difficult to distinguish residual disease from scar or normal tissue. When the evaluation of CR depends on this determination, it is recommended that the residual lesion be further investigated by fine needle aspirate/biopsy to confirm the CR status.
If a lesion disappears and reappears at a subsequent timepoint it should continue to be measured. However, the patient's response at the point in time when the lesion reappears will depend upon the status of his/her other lesions. For example, if the patient's tumor had reached a CR status and the lesion reappeared, then the patient would be considered PD at the time of reappearance. In contrast, if the tumor status was a PR or SD and one lesion which had disappeared then reappears, its maximal diameter should be added to the sum of the remaining lesions for a calculated response: in other words, the reappearance of an apparently ‘disappeared’ single lesion amongst many which remain is not in itself enough to qualify for PD: that requires the sum of all lesions to meet the PD criteria.

### Confirmation Measurement/Duration of Response

Confirmation of CR and PR is required and must occur no fewer than 4 weeks after initial documentation of CR or PR. If CR is pending confirmation and is designated at an assessment followed by 1 or more NE assessments, and/or PR assessments such that the Target Lesion Response is CR and the Non-Target Lesion Response is NE, CR may be confirmed thereafter if Non-Target Lesion Response returns to CR. Similarly, if a PR is pending confirmation and is designated at an assessment followed by 1 or more NE and/or SD assessments, PR may be confirmed thereafter. Subsequent Target Lesion Responses following a CR are limited to CR, PD or NE; PD for target lymph nodes is met only if any lymph node target lesion reaches a short axis measurement of 15 mm.

### ECOG Performance and NYHA Classification

ECOG Performance Status Scale

| Grade | Descriptions |
|---|---|
| 0 | Fully active, able to carry on all pre-disease performance without restriction. |
| 1 | Restricted in physically strenuous activity, but ambulatory and able to carry out work of a |
| | light or sedentary nature (e.g., light housework, office work). |
| 2 | Ambulatory and capable of all self-care, but unable to carry out any work activities. Up and |
| | about more than 50% of waking hours. |
| 3 | Capable of only limited self-care, confined to bed or chair more than 50% of waking hours. |
| 4 | Completely disabled. Cannot carry on any self-care. Totally confined to bed or chair. |
| 5 | Dead. |

Source: Oken et al, 1982; [0001]
ECOG = Eastern Cooperative Oncology Group;

### New York Heart Association Functional Classification

Class I No limitation of physical activity. Ordinary physical activity does not cause undue fatigue, palpitation or dyspnea.

Class II Slight limitation of physical activity. Comfortable at rest, but ordinary physical activity results in fatigue, palpitation or dyspnea.

Class III Marked limitation of physical activity. Comfortable at rest, but less than ordinary activity causes fatigue, palpitation or dyspnea.

Class IV Unable to carry out any physical activity without discomfort. Symptoms of cardiac insufficiency may be present even at rest. If any physical activity is undertaken, discomfort is increased.

### Example 3—Pharmacokinetic Analysis of 960 mg, 360 mg, 180 mg, and 240 mg Sotorasib

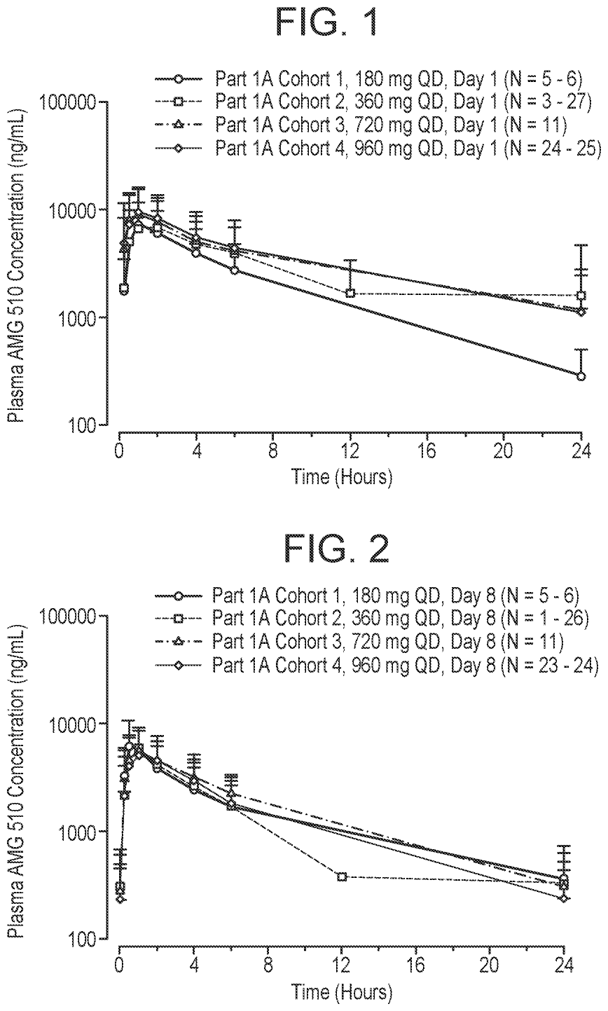

Preliminary pharmacokinetic (PK) data are available for subjects with advanced solid tumors with the specific KRAS G12C mutation, with doses ranging from 180 to 960 mg PO QD (Study 20170543; https://clinicaltrials.gov/ct2/show/NCT03600883; CodeBreaK 100). Dose-related increases in exposure on day 1 from 180 to 960 mg PO QD were observed. Increases in exposure were less than dose-proportional on day 1. There was no accumulation with multiple PO QD dosing for 8 days. The change in exposure from 180 to 960 mg PO QD was less than dose-proportional on day 8. Rapid absorption was observed with tₘₐₓ between 1 to 2 hours after PO administration. FIG. 1 shows the mean plasma concentration time profile after oral administration of 180, 360, 720, or 960 mg sotorasib on Day 1. FIG. 2 shows the concentrations after once daily dosing for 8 days (Day 8). The table below provides the pharmacokinetic parameters, where AUC₀₋₂₄ₕ is the area under the concentration-time curve from time 0 to 24 hr postdose; Cₘₐₓ is the maximum observed drug concentration during a dosing interval; t_{1/2,z} is the terminal elimination half-life; tₘₐₓ is the time to reach Cₘₐₓ. Data reported are presented as geometric mean (arithmetic CV %) except tₘₐₓ and t_{1/2}, which are reported as a median (range) and arithmetic mean (SD), respectively. Values are reported to three significant figures, except CV % and tₘₐₓ, which are reported to 0 decimal places and 2 significant figures, respectively.

### Pharmacokinetic Parameter

| | | tₘₐₓ | Cₘₐₓ | AUC_{0-24 h} | t_{1/2,z} |
|---|---|---|---|---|---|
| Dose | N | (hr) | (μg/mL) | (hr · μg/mL) | (hr) |
| 180 mg | | | | | |
| Day 1 | 6 | 1.0 (0.50-2.0) | 6.88 (51%) | 44.0 (56%) | 5.95 (1.08)^{a} |
| Day 8 | 6 | 0.75 (0.50-1.0) | 6.44 (67%) | 33.5 (85%) | 5.96 (2.76)^{b} |
| 360 mg | | | | | |
| Day 1 | 25 | 1.0 (0.50-24) | 5.86 (68%) | 56.8 (84%) | 6.56 (1.81)^{c} |
| Day 8 | 25 | 1.0 (0.25-4.0) | 5.97 (46%) | 37.4 (50%) | 5.71 (1.59)^{d} |
| 720 mg | | | | | |
| Day 1 | 11 | 1.0 (0.50-4.0) | 7.57 (59%) | 64.0 (68%) | 7.06 (1.59)^{e} |
| Day 8 | 11 | 1.0 (0.50-4.0) | 5.45 (50%) | 43.9 (49%) | 5.06 (1.24)^{f} |
| 960 mg | | | | | |
| Day 1 | 25 | 2.0 (0.25-6.0) | 8.33 (59%) | 68.0 (77%) | 6.00 (2.20)^{g} |
| Day 8 | 25 | 1.0 (0.50-24) | 4.91 (69%) | 32.7 (70%)^{h} | 5.19 (1.12)ⁱ |

ªN = 5;
^{b}N = 6;
^{c}N = 17;
^{d}N = 19;
^{e}N = 8;
^{f}N = 9;
^{g}N = 18;
^{h}N = 24;
ⁱN = 16;

### Example 4—Contraindication with Co-Administration of Sotorasib with Acid-Reducing Agents Under Fasted Conditions

This Phase 1, open-label, fixed-sequence study enrolled 14 healthy subjects. Subjects received 960 mg sotorasib on Day 1, 40 mg omeprazole once daily on Days 4 to 8, and 40 mg omeprazole followed by 960 mg sotorasib on Day 9. All doses were administered under fasted conditions. Blood samples for sotorasib PK were collected predose and up to 48 hours post-sotorasib dose. Sotorasib plasma PK parameters were estimated using non-compartmental methods.

Coadministration of sotorasib with omeprazole delayed sotorasib time to maximal plasma concentration (tₘₐₓ) by 0.75 hours. Mean terminal half-life (t_{1/2}) of sotorasib was similar following coadministration of sotorasib with omeprazole compared to administration of sotorasib alone. Geometric mean sotorasib AUC_{inf} (area under the curve from time zero to infinity) and Cₘₐₓ (maximal plasma concentration) following coadministration of sotorasib with omeprazole (17000 h*ng/mL and 3100 ng/mL, respectively) were lower compared to administration of sotorasib alone (29300 h*ng/mL and 7200 ng/mL, respectively). Sotorasib was safe and well tolerated when coadministered with 40 mg omeprazole or administered alone to healthy subjects.

Results indicated that coadministration of sotorasib with omeprazole, in the fasted state, decreased sotorasib AUC_{inf} by 42% and Cₘₐₓ by 57% compared with administration of sotorasib alone.

### Example 5—Contraindication with Co-Administration of Sotorasib with Acid-Reducing Agents Under Fed Conditions

This was a phase 1, open-label, fixed sequence, crossover, single-center study to explore mitigation strategies to limit the impact of acid-reducing agents on the exposure of sotorasib. This study evaluated the PK of sotorasib administered alone and in combination with famotidine or omeprazole in healthy men and women (a total of 14 subjects) underfed conditions. Subjects received a single dose of sotorasib on day 1, an evening dose of famotidine on day 3 (10 hours prior to sotorasib administration), a single dose of sotorasib on day 4 followed by another dose of famotidine 2 hours later, daily doses of omeprazole on day 6 through day 10, and a single dose of both omeprazole and sotorasib on day 11. All sotorasib administrations occurred following consumption of a standard calorie moderate fat meal. Blood was collected at predetermined timepoints to characterize plasma concentrations of sotorasib. Safety and tolerability monitoring was performed throughout the study.

A total of 15 healthy subjects (1 woman and 13 men) were enrolled in the study. Thirteen out of the 14 subjects received all treatments and completed the study.

Geometric least-square mean ratios of sotorasib AUC_{inf} and Cₘₐₓ were 0.622 and 0.654, respectively when comparing sotorasib coadministered with famotidine and sotorasib alone under fed conditions. Geometric least-square mean ratios of sotorasib AUC_{inf} and Cₘₐₓ were 0.430 and 0.349, respectively, when comparing sotorasib coadministered with omeprazole and sotorasib alone. Doses of 960 mg sotorasib were safe and well tolerated with coadministered with a single dose of 40 mg famotidine and following multiple daily dosing of 40 mg omeprazole under fed conditions to healthy subjects.

In summary, coadministration of a single dose of famotidine (H2 receptor antagonist) given 10 hours prior to and 2 hours after a single dose of sotorasib under fed conditions decreased sotorasib Cₘₐₓ by 35% and AUC by 38%. In addition, co-administration of repeat doses of omeprazole (PPI) with a single dose of sotorasib decreased sotorasib Cₘₐₓ by 65% and AUC by 57% under fed conditions.

### Example 6—Contraindication with Coadministration of Sotorasib with Strong CYP34A4 Inducers

This Phase 1, open-label, fixed-sequence study enrolled 14 healthy subjects. Each subject received 960 mg sotorasib on Days 1, 3 and 18, and 600 mg rifampin on Day 3 and Days 5 to 19. Blood samples for sotorasib PK were collected predose and up to 48 hours post-sotorasib dose. Sotorasib plasma PK parameters were estimated using non-compartmental methods.

### Results:

Geometric mean sotorasib AUC_{inf} (area under the curve from time zero to infinity) and Cₘₐₓ (maximal plasma concentration) following coadministration of single dose of rifampin with sotorasib (19600 h*ng/mL and 5340 ng/mL, respectively), were similar to those of sotorasib alone (25600 h*ng/mL and 6350 ng/mL, respectively). Geometric mean sotorasib AUC_{inf} and Cₘₐₓ following coadministration of multiple doses of rifampin with sotorasib (12400 h*ng/mL and 4110 ng/mL, respectively), were lower compared to those of sotorasib alone (25600 h*ng/mL and 6350 ng/mL, respectively).

Sotorasib was safe and well tolerated when coadministered with 600 mg rifampin or administered alone to healthy subjects. Single dose of rifampin did not have a clinically meaningful effect on sotorasib PK indicating sotorasib is not a substrate of OATP1B1. Multiple doses of rifampin decreased sotorasib AUC_{inf} by 51% and Cₘₐₓ by 35%, indicating sotorasib is a CYP3A4 substrate, consistent with in vitro data.

### Example 7—Contraindication with Coadministration of Sotorasib with CYP34A Substrates

This Phase 1, open-label, fixed-sequence study enrolled 5 subjects with previously untreated NSCLC who received a single, oral dose of 2 mg midazolam alone of day −1, 960 mg sotorasib orally on days 1 through 14, and a single oral dose of 2 mg midazolam at approximately the same time as an oral dose of 960 mg sotorasib on day 15. Blood samples for sotorasib PK were collected predose and up to 48 hours post-sotorasib dose. Sotorasib plasma PK parameters were estimated using non-compartmental methods.

Single dose plasma midazolam PK data were obtained from 5 subjects who received midazolam alone and midazolam coadministered with sotorasib following 14 days of multiple daily dosing of sotorasib. Results indicated that exposure to midazolam decreased when coadministered with sotorasib following multiple daily dosing with sotorasib. Coadministration of sotorasib with midazolam (a sensitive CYP3A4 substrate) decreased midazolam Cₘₐₓ by 48% and AUV by 53%.

### Example 8—Contraindication with Coadministration of Sotorasib and P-Gp Substrates

This Phase 1, open-label, fixed-sequence study enrolled 14 healthy subjects. Each subject received 0.5 mg digoxin on Day 1 and 960 mg sotorasib followed by 0.5 mg digoxin on Day 7. Blood samples for digoxin PK were collected predose and up to 144 hours post-digoxin dose. Samples were measured using validated high-performance liquid chromatography tandem mass spectrometry methods. PK parameters were estimated using non-compartmental methods. Safety and tolerability were monitored throughout the study.

Digoxin median time to maximal plasma concentration (tₘₐₓ) and mean terminal half-life (t_{1/2}) were similar following coadministration of digoxin with sotorasib compared to those of digoxin alone. Geometric mean digoxin AUC_{inf} (area under the curve from time zero to infinity) following coadministration of digoxin with sotorasib (40.3 h*ng/mL) was similar to that of digoxin alone (33.2 h*ng/mL). Geometric mean digoxin Cₘₐₓ (maximal plasma concentration) following coadministration of digoxin with sotorasib (3.64 ng/mL) was higher compared to that of digoxin alone (1.90 ng/mL). Single doses of 0.5 mg digoxin were safe and well tolerated when administered alone or coadministered with 960 mg sotorasib.

Results indicated that coadministration of digoxin with a single dose of sotorasib increased digoxin AUC_{inf} and Cₘₐₓ by approximately 21% and 91%, respectively, compared with digoxin alone.

### Example 9—Sotorasib in Combination with Panitumumab and Optionally FOLFIRI in Treatment-Naïve Patients with Metastatic Colorectal Cancer, Compared to Chemotherapy (FOLFOX or FOLFIRI) and Optionally Bevacizumab

The following example describes a phase 3, multicenter, randomized, open label, active-controlled study to evaluate efficacy and safety of sotorasib, panitumumab and FOLFIRI vs FOLFOX or FOLFIRI with or without bevacizumab-awwb in treatment-naïve metastatic colorectal cancer (mCRC) patients with KRAS G12C mutation.

The study will consist of a screening period, a treatment period, a safety follow up and long term follow up period. Approximately 450 treatment-naïve mCRC patients with KRAS G12C mutation will be enrolled and randomized 1:1 to receive either sotorasib, panitumumab and FOLFIRI or chemotherapy (FOLFOX or FOLFIRI) with or without bevacizumab-awwb.

Subjects will be stratified by region, number of organ sites of metastatic disease (1 vs >1) and age (<70 vs ≥70 yo).

Cycle 1 Day 1 will be defined as the first day subject receives study medication; Tumor assessment will be conducted (by MRI and/or CT) at baseline, at 8 weeks+/−1 week intervals until BICR assessed progression, start of another anti-cancer therapy, withdrawal of consent, loss to follow-up, or death, whichever occurs earliest. Tumor assessment and response will be determined by BICR using RECIST 1.1. Subjects may discontinue treatment because of disease progression as assessed by BICR, intolerance of treatment leading to treatment discontinuation, initiation of another anticancer therapy or withdrawal of consent.

In all subjects, information regarding the type and duration of subsequent therapies following disease progression, response to subsequent therapy, date of progression on subsequent therapy, and survival data will be collected. Subjects who discontinue treatment prior to RECIST 1.1 disease progression (e.g., due to unacceptable toxicity) will continue to be followed radiographically until disease progression, withdrawal of consent, or start of another anti-cancer therapy, and then further long-term follow up via phone or visit to clinic for assessment of survival and documentation of anticancer treatment. Subjects will be followed for 5 years after last subject randomized, or until withdrawal of consent, loss to follow-up, or subject death, whichever occurs first. The assessments to be conducted in this study are described below and will be carried out at the timepoints designated in the schedule of assessment (SOA):

Disease progression will be assessed using Response Evaluation Criteria in Solid Tumors, Version 1.1 (RECIST v1.1) as assessed by BICR.

Safety will be monitored by assessing serious and non-serious adverse events (AEs), safety laboratory tests, and vital signs. The incidence, nature and severity of all AEs will be graded according to the National Cancer Institute Common Terminology Criteria for Adverse Events, Version 5.0 (NCI CTCAE v5.0).

Laboratory safety tests will include hematology, blood chemistry, and urinalysis. Vital sign assessments will include blood pressure and pulse rate; additional vital signs will be collected only if clinically warranted.

Samples will be collected for sotorasib PK analyses.

Patient Reported Outcomes (PRO)/Quality of Life (QOL) assessments will be assessed.

Samples will be collected for tumor markers.

Additional plasma/blood/tissue biopsy samples will be collected for exploratory biomarkers for mechanisms of primary and secondary resistance.

### Inclusion Criteria:

Pathologically documented metastatic colorectal adenocarcinoma
Central confirmation of KRAS p.G12C mutation
Subjects must provide archived tumor tissue samples (formalin fixed, paraffin embedded [FFPE] sample collected within 5 years) or undergo a pre-treatment tumor biopsy prior to enrollment
Measurable disease per RECIST 1.1 criteria. Lesions previously radiated are not considered measurable unless they have progressed after radiation.
Age ≥18 years
Easter Cooperative Oncology Group (ECOG) Performance Status of ≤1
Life expectancy of >6 months
If prior adjuvant therapy was given for non-metastatic disease, it must be completed at least 6 months before the identification of metastatic disease
Adequate hematologic and end-organ function, defined as the following within 10 days prior to randomization:
ANC ≥1500 cells/NL (without granulocyte colony-stimulating factor support within 2 weeks of laboratory test used to determine eligibility)
Hemoglobin ≥9.0 g/dl (without transfusion within 2 weeks of laboratory test used to determine eligibility)
Platelet count ≥100,000/NI (without transfusion within 2 weeks of laboratory test used to determine eligibility)
Aspartate aminotransferase (AST) and alanine transaminase (ALT) ≤2.5 times the upper limit of normal (ULN)
Serum bilirubin ≤1.0×ULN
International normalized ratio (INR) and activated partial thromboplastin time ≤1.5×ULN
Serum creatinine ≤1.5×ULN or creatinine clearance >30 mL/min based on Cockcroft-Gault equation or by 24-hour urine collection
QTcF ≤470 msec in women and ≤450 msec in men
Ability to take oral medication and willing to record daily adherence to investigational product

### Exclusion Criteria:

Prior systemic therapy for metastatic disease
Active brain metastases. The phrase “active brain metastases” as used herein refers to a cancer that has spread from the original (primary, non-brain) tumor to the brain. Active brain metastases can be assessed by the presence of intracranial lesions. It is to be understood that while “metastases” is plural, patients exhibiting only one intracranial lesion under the criteria noted below is a patient who has “active brain metastases.” In some embodiments, a patient having active brain metastases has at least one measurable intracranial lesion >5 mm. In some embodiments, a patient having active brain metastases has at least one measurable intracranial lesion >5 mm but <10 mm. In some embodiments, a patient having active brain metastases has at least one measurable intracranial lesion >10 mm. A patient is not considered a patient with active brain metastases if the patient has had brain metastases resected or have received radiation therapy ending at least 4 weeks prior to study day 1 and are eligible if they meet all of the following criteria: a) residual neurological symptoms grade ≤2; b) on stable doses of dexamethasone or equivalent for at least 2 weeks, if applicable; and c) follow-up MRI performed within 28 days of Day 1 shows no progression or new lesions appearing. For determining the grade of any neurological symptom attributable to an intracranial lesion, see National Cancer Institute Common Terminology Criteria for Adverse Events v5.0 (NCI CTCAE) published Nov. 27, 2017 by the National Cancer Institute, incorporated herein by reference in its entirety.
Leptomeningeal disease.
Tumor is known to have BRAF V600E mutation.
Tumor is known to be MSI-H.
Unresolved toxicities from prior anti-tumor therapy, defined as not having resolved to CTCAE version 5.0 grade 0 or 1, or to levels dictated in the eligibility criteria with the exceptions of alopecia (any grade allowed), neuropathy (up to grade 2 allowed), or toxicities from prior anti-tumor therapy that are considered irreversible (defined as having been present and stable for >6 months), or endocrine AEs that are stably maintained on appropriate replacement therapy. For determining the grade of any neurological symptom attributable to an intracranial lesion, see National Cancer Institute Common Terminology Criteria for Adverse Events v5.0 (NCI CTCAE) published Nov. 27, 2017 by the National Cancer Institute, incorporated herein by reference in its entirety.
Anti-tumor therapy (chemotherapy, antibody therapy, molecular targeted therapy, retinoid therapy, hormonal therapy [except for subject with history of completely resected breast cancer with no active disease for over 5 years on long term adjuvant endocrine therapy], or investigational agent) within 4 weeks of study day 1; Please note that bisphosphonates or anti-RANKL antibody therapy is allowed if needed for management of hypercalcemia or for prevention of skeletal events.
Therapeutic or palliative radiation therapy within 2 weeks of study day 1. Subject must have recovered from radiotherapy related toxicity to CTCAE version 5.0 grade 1 or less with the exception of alopecia (any grade of alopecia allowed).
Currently receiving treatment with another investigational device or drug study, or less than 4 weeks since ending another investigational device or investigational agent(s).
Other investigational procedures are excluded
Previous treatment with a KRAS^{G12C} inhibitor.
Received cumulative radiation to >25% of bone marrow
Known dihydropyrimidine dehydrogenase deficiency
Known UDP-glucuronosyltransferase 1A1 (UGT1A1)*28 homozygosity or Gilbert's disease
Known sensitivity to any of the products or components to be administered during dosing.
Subject has required a dose reduction or dose delay of either 5-fluorouracil or irinotecan in any prior chemotherapy regimen in the past for toxicity to the investigator's knowledge.
Use of known cytochrome P450 (CYP) 3A4 sensitive substrates and P-gp substrates (with a narrow therapeutic window), within 14 days or 5 half-lives of the drug or its major active metabolite, whichever is longer, prior to study day 1 that was not reviewed and approved by the investigator and medical monitor.
Use of strong inducers of CYP3A4 (including herbal supplements such as St. John's wort) within 14 days or 5 half-lives (whichever is longer) prior to study day 1 that was not reviewed and approved by the principal investigator and medical monitor.
Use of known CYP3A4 or UGT1A1 inhibitors at least 1 week prior to starting irinotecan therapy unless reviewed and approved by the investigator and medical monitor.
Use of antiretroviral or viral drugs with potential to interact with study drug(s) unless reviewed and approved by principal investigator and medical monitor.
Female subjects of childbearing potential unwilling to use protocol specified method of contraception during treatment and for an additional:
   7 days after the last dose of sotorasib
   2 months after the last dose of panitumumab
   6 months after the last dose of FOLFIRI
   9 months after the last dose of FOLFOX
   6 months after the last dose of bevacizumab
Female subjects who are breastfeeding or who plan to breastfeed while on study through:
   7 days after the last dose of sotorasib
   2 months after the last dose of panitumumab
   6 months after the last dose of FOLFIRI
   3 months after the last dose of FOLFOX
   6 months after the last dose of bevacizumab
Female subjects of planning to become pregnant while on study through:
   7 days after the last dose of sotorasib
   2 months after the last dose of panitumumab
   6 months after the last dose of FOLFIRI
   9 months after the last dose of FOLFOX
   6 months after the last dose of bevacizumab
Female subject of childbearing potential with a positive pregnancy test assessed at screening or day 1 by a highly sensitive urine or serum pregnancy test.
Male subjects with a female partner of childbearing potential who are unwilling to practice sexual abstinence (refrain from heterosexual intercourse) or use contraception during treatment and for an additional:
   7 days after the last dose of sotorasib
   6 months after the last dose of FOLFIRI
   6 months after the last dose of FOLFOX
   6 months after the last dose of bevacizumab
Male subjects with a pregnant partner who are unwilling to practice abstinence or use a condom during treatment and for an additional:
   7 days after the last dose of sotorasib
   6 months after the last dose of FOLFIRI
   6 months after the last dose of FOLFOX
   6 months after the last dose of bevacizumab
Male subjects unwilling to abstain from donating sperm during treatment and for an additional:
   7 days after the last dose of sotorasib
   6 months after the last dose of FOLFIRI
   6 months after the last dose of FOLFOX
   6 months after the last dose of bevacizumab
Use of warfarin. Other anticoagulation may be allowed with medical monitor approval.
Known HIV with CD4+ T cell count <350 cells/NI
History of acquired immunodeficiency syndrome-defining opportunistic infections within the past 12 months
Known hepatitis B with a detectable viral load or hepatitis C with a detectable viral load
Malignancy other than CRC within 5 years prior to randomization, with the exception of those with a negligible risk of metastases or death and treated with expected curative outcome (such as adequately treated carcinoma in situ of the cervix, basal cell carcinoma, cutaneous squamous cell carcinoma, localized prostate cancer treated with curative intent, or ductal carcinoma in situ treated surgically with curative intent).
Major surgery within 28 days of study day 1
Significant uncontrolled concomitant disease that could affect compliance with protocol procedures or interpretation of results or that pose a risk to subject safety, in the opinion of the investigator
Significant gastrointestinal disorder that results in significant malabsorption, requirement for intravenous alimentation, or inability to take oral medication
History of interstitial pneumonitis or pulmonary fibrosis or evidence of interstitial pneumonitis or pulmonary fibrosis on baseline CT scan
History of evidence of severe acute respiratory syndrome coronavirus 2 (SARS-CoV-2) infection unless agreed upon with Medical Monitor and meeting the following criteria:
   Negative test for SARS-CoV-2 per local standard of care within 72 hours of first dose of sotorasib
   No acute symptoms of Coronavirus Disease 2019 (COVID19) disease within 10 days prior to first dose of sotorasib
Significant cardiovascular disease, such as New York Heath Association cardiac disease (Class II or greater), myocardial infarction within 6 months prior to randomization, unstable arrhythmias or unstable angina
Uncontrolled hypertension (SBP >140 or DB >90) for subjects where investigator intends to use bevacizumab if subject is randomized to the investigator's choice arm
Peripheral neuropathy ≥Grade 2 for subjects where investigator's choice is FOLFOX or FOLFOX and bevacizumab.
Therapeutic oral or intravenous antibiotics within 2 weeks prior to randomization. Prophylactic antibiotics are allowed.

### Study Products:

Sotorasib 960 mg or 240 mg oral, once daily
Panitumumab 6 mg/kg IV once every two weeks
Bevacizumab-awwb 5 mg/kg IV once every 2 weeks

### FOLFIRI:

Irinotecan 180 mg/m² IV once every two weeks
Leucovorin 400 mg/m² IV once every two weeks
5-fluorouracil 400 mg/m² IV bolus once every two weeks
5-fluorouracil 2400 mg/m² IVCI over 46-48 hours once every two weeks

### FOLFOX:

Oxaliplatin 85 mg/m² IV once every two weeks
Leucovorin 400 mg/m² IV once every two weeks
5-fluorouracil 400 mg/m² IV bolus once every two weeks
5-fluorouracil 2400 mg/m² IVCI over 46-48 hours once every two weeks

### Objectives/Endpoints:

| Objectives | Endpoints |
|---|---|
| Primary | |
| To compare progression-free survival (PFS) | PFS - defined as time form randomization |
| in treatment-naive subjects with KRAS | until disease progression or death from any |
| G12C mutated metastatic colorectal cancer | cause, whichever occurs first. Progression |
| (mCRC) received sotorasib, panitumumab | will be based on blinded independent central |
| and FOLFIRI vs. investigator's choice | review (BICR) of disease response per |
| chemotherapy (FOLFOX or FOLFIRI) with or | RECIST version 1.1 |
| without bevacizumab-awwb | |
| Secondary | |
| To evaluate other measures of efficacy of | Objective survival |
| sotorasib, panitumumab and FOLFIRI vs. | Objective response = complete response |
| investigator's choice of chemotherapy | [CR] + partial response [PR], assessed per |
| (FOLFOX or FOLFIRI) with or without | RECIST 1.1. Response will be assessed by |
| bevacizumab-awwb | BICR. CR and PR require confirmatory |
| | repeat assessment at least 4 weeks after |
| | the first detection of response |
| | Duration of response (DOR) |
| | Disease control (DCR) |
| | Time to response (TTR) |
| | Depth of response (best percent change |
| | from baseline in lesion sum diameters) |
| | PFS, ORR, DOR, DCR, TTR, depth of |
| | response based on investigator's |
| | assessment per RECIST 1.1 |
| To evaluate the safety and tolerability of | Incidence and severity of treatment- |
| sotorasib, panitumumab and FOLFIRI vs. | emergent adverse events, changes in vital |
| investigator's choice of chemotherapy | signs, and changes in clinical laboratory |
| (FOLFOX or FOLFIRI) with or without | tests |
| bevacizumab-awwb | |
| To characterize the pharmacokinetics (PK) | PK parameters of sotorasib including, but |
| of sotorasib | not limited to, maximum plasma |
| | concentration (Cₘₐₓ) and pre-dose (trough) |
| | concentrations, as applicable |
| To evaluate the impact of sotorasib, | Change form baseline at week 16 as |
| panitumumab and FOLFIRI vs. investigator's | assessed by the following questionnaires: |
| choice of chemotherapy (FOLFOX or | European Organization for Research |
| FOLFIRI) with or without bevacizumab- | and Treatment of Cancer (EORTC) Quality |
| awwb on patient-reported outcomes [PRO] | of Life Questionnaire (QLQ-30) for the 5 |
| | functional scales, 9 symptom scales, and |
| | the global health status/quality of life scale |
| | Colorectal cancer related symptoms |
| | measured by the EORTC Quality of Life |
| | Questionnaire Colorectal Cancer Module 29 |
| | (EORTC QLQ-CR29) questionnaire |
| | Visual analog scale (VAS) scores |
| | measured by the EuropQol-5D 5-level 5 |
| | (EQ-5D-5L) questionnaire |

### Example 10—Sotorasib in Combination with Panitumumab and Optionally FOLFIRI in Metastatic Colorectal Cancer Patients Compared to Treatment with FOLFIRI and Optionally Bevacizumab

The following example describes a phase 3, multicenter, randomized, open label, active-controlled study to evaluate efficacy and safety of sotorasib, panitumumab and FOLFIRI vs FOLFIRI with or without bevacizumab-awwb in previously treated metastatic colorectal cancer (mCRC) patients with KRAS G12C mutation.

The study will consist of a screening period, a treatment period, a safety follow up and long term follow up period. Approximately 350 previously treated mCRC patients with KRAS G12C mutation will be enrolled and randomized 1:1 to receive either sotorasib, panitumumab and FOLFIRI or FOLFIRI with or without bevacizumab-awwb.

Subjects will be stratified by region, number of organ sites of metastatic disease (1 vs >1) and age (<70 vs >70 yo).

Cycle 1 Day 1 will be defined as the first day subject receives study medication; Tumor assessment will be conducted (by MRI and/or CT) at baseline, at 8 weeks+/−1 week intervals until BICR assessed progression, start of another anti-cancer therapy, withdrawal of consent, loss to follow-up, or death, whichever occurs earliest. Tumor assessment and response will be determined by BICR using RECIST 1.1. Subjects may discontinue treatment because of disease progression as assessed by BICR, intolerance of treatment leading to treatment discontinuation, initiation of another anticancer therapy or withdrawal of consent.

In all subjects, information regarding the type and duration of subsequent therapies following disease progression, response to subsequent therapy, date of progression on subsequent therapy, and survival data will be collected. Subjects who discontinue treatment prior to RECIST 1.1 disease progression (e.g., due to unacceptable toxicity) will continue to be followed radiographically until disease progression, withdrawal of consent, or start of another anti-cancer therapy, and then further long-term follow up via phone or visit to clinic for assessment of survival and documentation of anticancer treatment. Subjects will be followed for 5 years after last subject randomized, or until withdrawal of consent, loss to follow-up, or subject death, whichever occurs first.

The assessments to be conducted in this study are described below and will be carried out at the timepoints designated in the schedule of assessment (SOA):
Disease progression will be assessed using Response Evaluation Criteria in Solid Tumors, Version 1.1 (RECIST v1.1) as assessed by BICR.
Safety will be monitored by assessing serious and non-serious adverse events (AEs), safety laboratory tests, and vital signs. The incidence, nature and severity of all AEs will be graded according to the National Cancer Institute Common Terminology Criteria for Adverse Events, Version 5.0 (NCI CTCAE v5.0).
Laboratory safety tests will include hematology, blood chemistry, and urinalysis. Vital sign assessments will include blood pressure and pulse rate; additional vital signs will be collected only if clinically warranted.
Samples will be collected for sotorasib PK analyses.
Patient Reported Outcomes (PRO)/Quality of Life (QOL) assessments will be assessed.
Samples will be collected for sotorasib PK analyses.
Samples will be collected for tumor markers.
Additional plasma/blood/tissue biopsy samples will be collected for exploratory biomarkers for mechanisms of primary and secondary resistance.

### Inclusion Criteria:

Pathologically documented metastatic colorectal adenocarcinoma
Central confirmation of KRAS G12C mutation previously identified by local testing
Subjects must provide archived tumor tissue samples (formalin fixed, paraffin embedded [FFPE] sample collected within 5 years) or undergo a pre-treatment tumor biopsy prior to enrollment
Measurable disease per RECIST 1.1 criteria. Lesions previously radiated are not considered measurable unless they have progressed after radiation.
Age ≥18 years
Easter Cooperative Oncology Group (ECOG) Performance Status of ≤1
Life expectancy of >6 months
Subject must have received one and only one prior line of systemic therapy for metastatic disease and progressed on or after that therapy. If prior adjuvant therapy was given for non-metastatic disease, it will count as a line of therapy for metastatic disease if metastatic disease is identified within 6 months of the end of adjuvant therapy. If tumor is MSI-H, subject must have received a checkpoint inhibitor for metastatic disease if available in the region or country and the subject does not have a medical contraindication to the therapy.
Adequate hematologic and end-organ function, defined as the following within 10 days prior to randomization:
ANC ≥1500 cells/NL (without granulocyte colony-stimulating factor support within 2 weeks of laboratory test used to determine eligibility)
Hemoglobin ≥9.0 g/dl (without transfusion within 2 weeks of laboratory test used to determine eligibility)
Platelet count ≥100,000/NI (without transfusion within 2 weeks of laboratory test used to determine eligibility)
Aspartate aminotransferase (AST) and alanine transaminase (ALT) ≤2.5 times the upper limit of normal (ULN)
Serum bilirubin ≤1.0×ULN
International normalized ratio (INR) and activated partial thromboplastin time ≤1.5×ULN
Serum creatinine ≤1.5×ULN or creatinine clearance >30 mL/min based on Cockcroft-Gault equation or by 24-hour urine collection
QTcF ≤470 msec in women and ≤450 msec in men
Ability to take oral medication and willing to record daily adherence to investigational product

### Exclusion Criteria:

Active brain metastases. The phrase “active brain metastases” as used herein refers to a cancer that has spread from the original (primary, non-brain) tumor to the brain. Active brain metastases can be assessed by the presence of intracranial lesions. It is to be understood that while “metastases” is plural, patients exhibiting only one intracranial lesion under the criteria noted below is a patient who has “active brain metastases.” In some embodiments, a patient having active brain metastases has at least one measurable intracranial lesion >5 mm. In some embodiments, a patient having active brain metastases has at least one measurable intracranial lesion >5 mm but <10 mm. In some embodiments, a patient having active brain metastases has at least one measurable intracranial lesion >10 mm. A patient is not considered a patient with active brain metastases if the patient has had brain metastases resected or have received radiation therapy ending at least 4 weeks prior to study day 1 and are eligible if they meet all of the following criteria: a) residual neurological symptoms grade ≤2; b) on stable doses of dexamethasone or equivalent for at least 2 weeks, if applicable; and c) follow-up MRI performed within 28 days of Day 1 shows no progression or new lesions appearing. For determining the grade of any neurological symptom attributable to an intracranial lesion, see National Cancer Institute Common Terminology Criteria for Adverse Events v5.0 (NCI CTCAE) published Nov. 27, 2017 by the National Cancer Institute, incorporated herein by reference in its entirety.
Leptomeningeal disease.
Tumor is known to have BRAF V600E mutation.
Unresolved toxicities from prior anti-tumor therapy, defined as not having resolved to CTCAE version 5.0 grade 0 or 1, or to levels dictated in the eligibility criteria with the exceptions of alopecia (any grade allowed), neuropathy (up to grade 2 allowed), or toxicities from prior anti-tumor therapy that are considered irreversible (defined as having been present and stable for >6 months), or endocrine AEs that are stably maintained on appropriate replacement therapy.
Anti-tumor therapy (chemotherapy, antibody therapy, molecular targeted therapy, retinoid therapy, hormonal therapy [except for subject with history of completely resected breast cancer with no active disease for over 5 years on long term adjuvant endocrine therapy], or investigational agent) within 4 weeks of study day 1; Please note that bisphosphonates or anti-RANKL antibody therapy is allowed if needed for management of hypercalcemia or for prevention of skeletal events.
Therapeutic or palliative radiation therapy within 2 weeks of study day 1. Subjects must have recovered from radiotherapy related toxicity to CTCAE version 5.0 grade 1 or less with the exception of alopecia (any grade of alopecia allowed).
Currently receiving treatment with another investigational device or drug study, or less than 4 weeks since ending another investigational device or investigational agent(s).
Other investigational procedures are excluded
Previous treatment with a KRAS^{G12C} inhibitor.
Received cumulative radiation to >25% of bone marrow
Known dihydropyrimidine dehydrogenase deficiency
Known UDP-glucuronosyltransferase 1A1 (UGT1A1)*28 homozygosity or Gilbert's disease
Known sensitivity to any of the products or components to be administered during dosing.
Subject has required a dose reduction or dose delay of either 5-fluorouracil or irinotecan in any prior chemotherapy regimen in the past for toxicity to the investigator's knowledge.
Prior treatment with irinotecan given for metastatic disease.
Use of known cytochrome P450 (CYP) 3A4 sensitive substrates and P-gp substrates (with a narrow therapeutic window), within 14 days or 5 half-lives of the drug or its major active metabolite, whichever is longer, prior to study day 1 that was not reviewed and approved by the investigator and medical monitor.
Use of strong inducers of CYP3A4 (including herbal supplements such as St. John's wort) within 14 days or 5 half-lives (whichever is longer) prior to study day 1 that was not reviewed and approved by the principal investigator and medical monitor.
Use of known CYP3A4 or UGT1A1 inhibitors at least 1 week prior to starting irinotecan therapy unless reviewed and approved by principal investigator and medical monitor.
Use of antiretroviral or viral drugs with potential to interact with study drug(s) unless reviewed and approved by principal investigator and medical monitor.
Female subjects of childbearing potential unwilling to use protocol specified method of contraception during treatment and for an additional:
   7 days after the last dose of sotorasib
   2 months after the last dose of panitumumab
   6 months after the last dose of FOLFIRI
   6 months after the last dose of bevacizumab
Female subjects who are breastfeeding or who plan to breastfeed while on study through:
   7 days after the last dose of sotorasib
   2 months after the last dose of panitumumab
   7 days after the last dose of FOLFIRI
   6 months after the last dose of bevacizumab
Female subjects planning to become pregnant while on study through:
   7 days after the last dose of sotorasib
   2 months after the last dose of panitumumab
   6 months after the last dose of FOLFIRI
   6 months after the last dose of bevacizumab
Female subjects of childbearing potential with a positive pregnancy test assessed at screening or day 1 by a highly sensitive urine or serum pregnancy test.
Male subjects with a female partner of childbearing potential who are unwilling to practice sexual abstinence (refrain from heterosexual intercourse) or use contraception during treatment and for an additional:
   7 days after the last dose of sotorasib
   6 months after the last dose of FOLFIRI
   6 months after the last dose of bevacizumab
Male subjects with a pregnant partner who are unwilling to practice abstinence or use a condom during treatment and for an additional:
   7 days after the last dose of sotorasib
   6 months after the last dose of FOLFIRI
   6 months after the last dose of bevacizumab
Male subjects unwilling to abstain from donating sperm during treatment and for an additional:
   7 days after the last dose of sotorasib
   6 months after the last dose of FOLFIRI
   6 months after the last dose of bevacizumab
Use of warfarin. Other anticoagulation may be allowed with medical monitor approval.
Known HIV with CD4+ T cell count <350 cells/NI
History of acquired immunodeficiency syndrome-defining opportunistic infections within the past 12 months
Known hepatitis B with a detectable viral load or hepatitis C with a detectable viral load
Malignancy other than CRC within 5 years prior to randomization, with the exception of those with a negligible risk of metastases or death and treated with expected curative outcome (such as adequately treated carcinoma in situ of the cervix, basal cell carcinoma, cutaneous squamous cell carcinoma, localized prostate cancer treated with curative intent, or ductal carcinoma in situ treated surgically with curative intent).
Major surgery within 28 days of study day 1
Significant uncontrolled concomitant disease that could affect compliance with protocol procedures or interpretation of results or that pose of risk to subject safety, in the opinion of the investigator
Significant gastrointestinal disorder that results in significant malabsorption, requirement for intravenous alimentation, or inability to take oral medication
History of interstitial pneumonitis or pulmonary fibrosis or evidence of interstitial pneumonitis or pulmonary fibrosis on baseline CT scan
History of evidence of severe acute respiratory syndrome coronavirus 2 (SARS-CoV-2) infection unless agreed upon with Medical Monitor and meeting the following criteria:
   Negative test for SARS-CoV-2 per local standard of care within 72 hours of first dose of sotorasib
   No acute symptoms of Coronavirus Disease 2019 (COVID19) disease within 10 days prior to first dose of sotorasib
Significant cardiovascular disease, such as New York Heath Association cardiac disease (Class II or greater), myocardial infarction within 6 months prior to randomization, unstable arrhythmias or unstable angina
Uncontrolled hypertension (SBP >140 or DB >90) for subjects where investigator intends to use bevacizumab if subject is randomized to the investigator's choice arm
Therapeutic oral or intravenous antibiotics within 2 weeks prior to randomization. Prophylactic antibiotics are allowed.

### Study Products:

Sotorasib 960 mg or 240 mg oral, daily
Panitumumab 6 mg/kg IV once every two weeks
Bevacizumab-awwb 5 mg/kg IV once every 2 weeks

### FOLFIRI:

Irinotecan 180 mg/m² IV once every two weeks
Leucovorin 400 mg/m² IV once every two weeks
5-fluorouracil 400 mg/m² IV bolus once every two weeks
5-fluorouracil 2400 mg/m² IVCI over 46-48 hours once every two weeks

### Objectives/Endpoints:

| Objectives | Endpoints |
|---|---|
| Primary | |
| To compare overall survival (OS) in | Overall survival - defined as time from |
| previously treated subjects with KRAS G12C | randomization until death from any cause |
| mutated mCRC receiving sotorasib, | |
| panitumumab and FOLFIRI vs FOLFIRI with | |
| or without bevacizumab-awwb | |
| Key secondary | |
| To compare progression-free survival (PFS) | PFS - defined as time form randomization |
| in previously treated subject with KRAS | until disease progression or death from any |
| G12C mutated metastatic colorectal cancer | cause, whichever occurs first. Progression |
| (mCRC) receiving sotorasib sotorasib, | will be based on blinded independent central |
| panitumumab and FOLFIRI vs. FOLFIRI) | review (BICR) of disease response per |
| with or without bevacizumab-awwb | RECIST version 1.1 |
| To compare objective response rate (ORR) | Objective response = complete response |
| in previously treated subjects with KRAS | [CR] + partial response [PR], assessed per |
| G12C mutated mCRC receiving sotorasib, | RECIST 1.1. Response will be assessed by |
| panitumumab and FOLFIRI vs. FOLFIRI | BICR. CR and PR require confirmatory |
| with or without bevacizumab-awwb | repeat assessment at least 4 weeks after |
| | the first detection of response |
| Secondary | |
| To characterize the pharmacokinetics (PK) | PK parameters of sotorasib including, but |
| of sotorasib | not limited to, maximum plasma |
| | concentration (Cₘₐₓ) and pre-dose (trough) |
| | concentrations, as applicable |
| To evaluate other measures of efficacy of | Duration of response (DOR) |
| sotorasib, panitumumab and FOLFIRI vs. | Disease control (DCR) |
| FOLFIRI with or without bevacizumab-awwb | Time to response (TTR) |
| | Depth of response (best percent change |
| | from baseline in lesion sum diameters) |
| | PFS, ORR, DOR, DCR, TTR, depth of |
| | response based on investigator's |
| | assessment per RECIST 1.1 |
| To evaluate the safety and tolerability of | Incidence and severity of treatment- |
| sotorasib, panitumumab and FOLFIRI vs. | emergent adverse events, changes in vital |
| FOLFIRI with or without bevacizumab-awwb | signs, and changes in clinical laboratory |
| | tests |
| To evaluate the impact of sotorasib, | Change form baseline at week 12 as |
| panitumumab and FOLFIRI vs. FOLFIRI | assessed by the following questionnaires: |
| with or without bevacizumab-awwb on | European Organization for Research |
| patient-reported outcomes [PRO] | and Treatment of Cancer (EORTC) Quality |
| | of Life Questionnaire (QLQ-30) for the 5 |
| | functional scales, 9 symptom scales, and |
| | the global health status/quality of life scale |
| | Colorectal cancer related symptoms |
| | measured by the EORTC Quality of Life |
| | Questionnaire Colorectal Cancer Module 29 |
| | (EORTC QLQ-CR29) questionnaire |
| | Visual analog scale (VAS) scores |
| | measured by the EuropQol-5D 5-level 5 |
| | (EQ-5D-5L) questionnaire |

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### REFERENCES

Albert et al., 2007, Nat. Methods, 4:903-905.
Alizadeh et al., 1996, Nat. Genet., 14:457-460.
Beers and Nederlof, 2006, Breast Cancer Res., 8(3):210.
Bertone et al., 2006, Genome Res. 16(2):271-281.
Canon et al., 2019, Nature 575(7781), 217.
Cecil Textbook of Medicine, pp. 2317-2341, W.B. Saunders & Co. (1985)
Cerami et al. 2012, Cancer Discov., 2(5), 401.
Chung et al., 2004, Genome Res., 14(1):188-196.
CAMPTOSAR® US Prescribing Information, Pharmacia and Upjohn Co., Division of Pfizer, Inc., NY, NY 10017 (revision January 2022)
Cully M, Downward J., 2008, Cell, 133:1292.
Dalma-Weiszhausz et al. 2006 Methods Enzymol., 410:3-28.
Eisenhauer et al., 2009, Eur. J. Cancer, 45:228-247.
Fakih et al., 2021, Abstract #3245: European Society for Medical Oncology (ESMO), Sep. 16-21, 2021.
Fakih et al., 2021, ePoster #3245: European Society for Medical Oncology (ESMO), Sep. 16-21, 2021.
Forshew et al., 2012, Sci Transl Med., 4:136ra68.
Gao et al., 2013, Science Signaling, 6(269), p11.
Haber and Velculescu, 2014, Cancer Discov., 4:650-61.
Hong et al., 2020, N. Engl. J. Med., 383, 1207.
Hughes et al., 2001, Nat. Biotechnol., 19(4):342-347.
Irizarry, 2003, Nucleic Acids Res., 31:e15.
Janes et al., 2018, Cell., 172(3):578-589.
Jasmine et al., 2012, PLoS One, 7(2):e31968.
Kim et al., 2006, Carcinogenesis, 27(3):392-404.
Kinde et al., 2011, Proc Natl Acad Sci USA; 108:9530-5.
Kuboki et al., 2022, European Society for Medical Oncology (ESMO), Sep. 9-13, 2022.
Kumar et al., 2012, J. Pharm. Bioallied Sci., 4(1):21-26.
Laere et al., 2009, Methods Mol. Biol., 512:71-98.
Lanman et al., 2020, J. Med. Chem., 63, 52.
Lin et al., 2010, BMC Genomics, 11:712.
Liu et al., 2017, Biosens Bioelectron, 92:596-601.
Lodes et al., 2009, PLoS One, 4(7):e6229.
LUMAKRAS® US Prescribing Information, Amgen Inc., Thousand Oaks, California, 91320 (revision May 2021)
Mackay et al. 2003, Oncogene, 22:2680-2688.
Mao et al., 2007, Curr. Genomics 8(4):219-228.
McDonald et al., 2017, Cell, 170(3):577-592.
Michels et al., 2007, Genet. Med., 9:574-584.
Mockler and Ecker, 2005, Genomics, 85(1):1-15.
National Cancer Institute Common Terminology Criteria for Adverse Events v5.0 (NCI CTCAE) published Nov. 27, 2017 by the National Cancer Institute
Oken et al., 1982, Am J Clin Oncol., 5(6):649-655.
Ostrem et al., 2013, Nature, 503:548-551.
Ostrem and Shokat, 2016, Nature Rev Drug Discov., 15(11):771-785.
Patricelli et al., 2016, Cancer Discovery, 6:316-329.
Peeters et al., 2010, J Clin Oncol., 28:4706-13
Pinkel et al., 2005, Nat. Genetics, 37:S11-S17.
Simanshu et al., 2017, Cell, 170:17-33.
Thomas et al., 2005, Genome Res., 15(12):1831-1837.
Thompson et al., 2012, PLoS ONE, 7:e31597.
VECTIBIX® US Prescribing Information. Amgen Inc., Thousand Oaks, California, 91320, revision August 2021
Wang et al., 2012, Cancer Genet., 205(7-8):341-55.
Wei et al., 2008, Nucleic Acids Res., 36(9):2926-2938.
WO2021/142026
WO2021/224867
Xie et al., 2017, Front Pharmacol., 8:823.
Zubrod et al., 1960, J Chronic Disease, 11:7-33.

What is claimed is:

1. A method of treating cancer comprising a KRAS G12C mutation in a patient comprising administering to the patient (a) sotorasib and (b) an anti-epidermal growth factor receptor (EGFR) antibody in amounts effective to treat the cancer.

2. The method of claim 1, comprising administering 960 mg sotorasib to the patient daily.

3. The method of claim 1, comprising administering 240 mg sotorasib to the patient daily.

4. The method of any one of claims 1-3, comprising administering sotorasib to the patient once daily.

5. The method of any one of claims 1-3, comprising administering sotorasib to the patient twice daily.

6. The method of any one of claims 1-5, wherein the anti-EGFR antibody is panitumumab.

7. The method of claim 6, comprising administering 6 mg/kg panitumumab to the patient.

8. The method of claim 6 or claim 7, comprising administering to the patient
(a) 960 mg sotorasib daily; and
(b) 6 mg/kg panitumumab via IV administration every two weeks.

9. The method of claim 6 or claim 7, comprising administering to the patient
(a) 240 mg sotorasib daily; and
(b) 6 mg/kg panitumumab via IV administration every two weeks.

10. The method of any one of claims 1-13, further comprising administering (c) irinotecan, (d) 5-FU and (e) leucovorin or levoleucovorin to the patient.

11. The method of claim 10, comprising administering 400 mg/m² leucovorin via IV administration to the patient.

12. The method of claim 10, comprising administering 200 mg/m² levoleucovorin via IV administration to the patient.

13. The method of any one of claims 10-12, comprising administering 180 mg/m² irinotecan via IV administration to the patient.

14. The method of any one of claims 10-13, comprising administering 400 mg/m² 5-FU via IV administration to the patient.

15. The method of claim 10, comprising administering via IV administration 180 mg/m² irinotecan, 400 mg/m² leucovorin, and 400 mg/m² 5-FU to the patient every two weeks IV bolus and 2400 mg/m² 5-FU IV continuous infusion over 46-48 hours to the patient.

16. The method of claim 10, comprising administering via IV administration 180 mg/m² irinotecan, 200 mg/m² levoleucovorin, and 400 mg/m² 5-FU IV bolus and 2400 mg/m² 5-FU IV continuous infusion over 46-48 hours to the patient every two weeks.

17. The method of any one of claims 1-16, wherein the cancer is a solid tumor.

18. The method of any one of claims 1-17, wherein the cancer is non-small cell lung cancer (NSCLC).

19. The method of any one of claims 1-17, wherein the cancer is metastatic pancreatic cancer.

20. The method of any one of claims 1-17, wherein the cancer is colorectal cancer.

21. The method of any one or claims 1-17, wherein the cancer is metastatic colorectal cancer (mCRC).

22. The method of any one of claims 1-36, wherein the patient has received at least one prior systemic cancer therapy.

23. The method of any one of claims 1-36, wherein the patient has received at least two prior systemic cancer therapies.

24. The method of claim 22 and claim 23, wherein the systemic cancer therapy is a therapy comprising administering to the patient fluoropyrimidine, irinotecan, and oxaliplatin.

25. The method of any one of claims 21-24, wherein the mCRC is determined to be MSI-H and the systemic cancer therapy is a therapy comprising administering to the patient a checkpoint inhibitor.

26. The method of any one of claims 21-25, wherein the mCRC comprises a BRAF V600E mutation and the systemic cancer therapy is a therapy comprising administering to the patient encorafenib and cetuximab.

27. The method of any one of claims 21-26, wherein the patient exhibits an ECOG performance status of equal or less than 2.

28. The method of any one of claims 21-27, wherein the patient does not have active brain metastases.

29. The method of any one of claims 22-28, wherein the systemic therapy is not a therapy comprising administering to the patient a KRAS^{G12C} inhibitor.

30. The method of any one of claims 1-21, wherein the patient has not received a prior systemic cancer therapy.

31. The method of claim 30, wherein the patient does not have active brain metastases.

32. The method of claim 30 or claim 31, wherein the mCRC does not comprise a BRAF V600E mutation.

33. The method of any one of claims 30-32, wherein the mCRC is determined not to be MSI-H.

34. The method of any one of claims 30-33, wherein the systemic therapy is a therapy comprising administering to the patient a KRAS^{G12C} inhibitor.

35. The method of any one of claims 30-34, wherein the patient exhibits an ECOG performance status of equal or less than 1.

36. The method of any one of claims 1-21, wherein the patient has received one prior systemic cancer therapy.

37. The method of claim 36, wherein if the cancer is determined to be MSI-H, then the systemic cancer therapy is a checkpoint inhibitor.

38. The method of claim 36 or claim 37, wherein the patient has received the systemic cancer therapy and progressed on or after said therapy.

39. The method of any one of claims 36-38, wherein the systemic therapy is not a therapy comprising administering to the patient a KRAS^{G12C} inhibitor.

40. The method of any one of claims 36-38, wherein the systemic therapy is not a therapy comprising administering irinotecan.

41. The method of any one of claims 36-40, wherein the patient exhibits an ECOG performance status of equal or less than 1.

42. The method of any one of claims 36-41, wherein the patient does not have active brain metastases.

43. The method of any one of claims 36-42, wherein the mCRC does not comprise a BRAF V600E mutation.

44. The method of any one of claims 1-43, wherein the patient exhibits at least a stable disease (SD) after 1, 3, or 6 months of sotorasib and panitumumab therapy, as measured by RECIST 1.1 protocol.

45. The method of any one of claims 1-43, wherein the patient exhibits at least a partial response (PR) after 1, 3, or 6 months of sotorasib and panitumumab therapy, as measured by RECIST 1.1 protocol.

46. The method of any one of claims 1-45, wherein the patient is in further need of treatment with an acid-reducing agent.

47. The method of claim 46, wherein the acid-reducing agent is a proton pump inhibitor (PPI), a H2 receptor antagonist (H2RA), or a locally acting antacid.

48. The method of claim 46 or claim 47, wherein the acid-reducing agent is a locally acting antacid, and wherein sotorasib is administered about 4 hours before or about 10 hours after the locally acting antacid.

49. The method of any one of claims 1-48, wherein the patient is in further need of treatment with a proton pump inhibitor (PPI) or H2 receptor antagonist (H2RA).

50. The method of claim 49, wherein the patient is not administered a PPI or a H2RA in combination with sotorasib.


SEQUENCE LISTING (10 sequences)

SEQ ID NO: 1 - protein, 7 aa, synthetic construct
  REGION 1..7: note = Synthetic Polypeptide- HCDR1
SGDYYWT

SEQ ID NO: 2 - protein, 16 aa, synthetic construct
  REGION 1..16: note = Synthetic Polypeptide - HCDR2
HIYYSGNTNYNPSLKS

SEQ ID NO: 3 - protein, 9 aa, synthetic construct
  REGION 1..9: note = Synthetic Polypeptide- HCDR3
DRVTGAFDI

SEQ ID NO: 4 - protein, 119 aa, synthetic construct [sequence omitted]

SEQ ID NO: 5 - protein, 444 aa, synthetic construct
  REGION 1..444: note = Synthetic Polypeptide -Heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 6 - protein, 11 aa, synthetic construct
  REGION 1..11: note = Synthetic Polypeptide- LCDR1
QASQDISNYLN

SEQ ID NO: 7 - protein, 7 aa, synthetic construct
  REGION 1..7: note = Synthetic Polypeptide - LCDR2
DASNLET

SEQ ID NO: 8 - protein, 9 aa, synthetic construct
  REGION 1..9: note = Synthetic Polypeptide - LCDR3
QHFDHLPLA

SEQ ID NO: 9 - protein, 110 aa, synthetic construct
  REGION 1..110: note = Synthetic Polypeptide- Light variable
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTV

SEQ ID NO: 10 - protein, 214 aa, synthetic construct
  REGION 1..214: note = Synthetic Polypeptide- Light chain
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC